(19) United States
(12) Patent Application Publication
(10) Pub. No.: US 2026/0363555 A1
(43) Pub. Date: Nov. 19, 2026

(54) SYSTEMS AND METHOD FOR DETECTING EMERGENT PROPERTIES AND IMPROVING HEALTH OUTCOMES

(71) Applicant: EMERJA CORPORATION, Monte Sereno, CA (US)

(72) Inventors: Guy Miller, Monte Sereno, CA (US); Daniel Hansen, Santa Cruz, CA (US)

(21) Appl. No.: 19/133,658

(22) PCT Filed: Dec. 11, 2023
(86) PCT No.: PCT/US2023/083441

§ 371 Date: May 28, 2025

Related U.S. Application Data
(60) Provisional application No. 63/387,276, filed on Dec. 13, 2022.
(60) Provisional application No. 63/486,930, filed on Feb. 24, 2023.
(60) Provisional application No. 63/488,892, filed on Mar. 7, 2023.

(51) Int. Cl. A61B 5/01; A61B 5/00

(52) U.S. Cl. CPC A61B 5/01; A61B 5/486; A61B 5/7275; A61B 5/7282; A61B 5/746; A61B 5/747; A61B 2560/0252; A61B 2562/04

(57) ABSTRACT

Systems are described to identify at least one recognizable feature associated with a health state of a subject, in which the system comprises (i) at least one sensor for measuring a heat flux of said subject and for generating a data stream therefrom, where the data stream includes a plurality of heat flux measurements; and (ii) a processor configured to receive the data stream and recognize said at least one recognizable feature associated with said health state. Methods are described for (a) assessing resilience of thermoregulation in a subject and generating an alter or a recommendation to engage in an intervention as to the subject based on the assessed resilience of thermoregulation, and (b) identifying recognizable features associated with health states of a subject, in which the methods comprise using at least one sensor to measure a heat flux of the subject and generating a data stream therefrom, where the data stream includes a plurality of heat flux measurements, and employing a processor may be used to receive the data stream and recognize the at least one recognizable feature associated with the health state.

### CROSS-REFERENCE TO RELATED APPLICATIONS

This application claims priority to U.S. Provisional Application No. 63/387,276, filed Dec. 13, 2022; U.S. Provisional Application No. 63/486,930, filed Feb. 24, 2023; and U.S. Provisional Application No. 63/488,892, filed Mar. 7, 2023, the content of each of which is incorporated by reference in its entirety.

### BACKGROUND

### Field of the Disclosed Technology

The disclosed technology generally relates to systems for, wearable devices for, and methods of detecting recognizable features from measurements of emergent properties of a complex adaptive system, such as a biological system, an organism, or for example a human, or a non-biological system.

### Description of the Related Art

The current approach to solving problems in the biological sciences is a bottoms-up approach often referred to as “precision biology.” In its most generalized form precision biology couples machine learning with a detailed measurement (“-omics”) of biological parts to ascribe function to parts. This approach is also often referred to as “precision medicine,” especially when applied to the discovery, development and delivery of healthcare solutions.

Precision biology is based on the concept that knowledge gaps are due to a lack of understanding of “parts,” and that detailed measurement and analysis will fill in these knowledge gaps. For example, a basic tool of modern biological sciences is organic chemistry, the study of carbon-containing molecules, and carbon bonded to other key atoms. This is, at least in part, because the genetic, signaling, and structural molecules of living systems consist largely of carbon atoms. Under the precision biology paradigm, function and prediction of function is therefore sought through quantifying these organic chemical attributes in greater detail. However, the art generally fails to recognize that many characteristics of biological systems do not lend themselves to the precision biology paradigm. One such example, where the precision biology paradigm fails, is in the measurement and prediction of emergent properties of complex adaptative (biological) systems. Emergent properties are properties of a system not found in a part, or readily deducible from a detailed inventory and analysis of the parts contained within a system. The precision biology approach—which today has risen to the level of a paradigm—has a blind spot with respect to emergent properties; this blind spot substantially limits advances in the fields of biology and medicine.

A historic perspective sheds light on the manifold advantages of the current technology, even as all past and current understandings of biological systems, and of how to monitor them and maintain or improve health neither anticipate nor render obvious the systems, methods, and uses of the current technology. Known devices for and methods of monitoring biological systems, and known systems for maintaining or improving health are inadequate. For example, known wearable devices generally exist in the off-the-shelf market; however, many patients and users are excluded from that market.

The measurement of human biological systems to understand their function can be traced as far back as Hippocrates in ~450 BC. Hippocrates is credited for separating medicine from religion in human biological systems, and thus establishing a physical basis for measuring and diagnosing disease and developing prognosticators. The notion that human biology could be understood through the prism of physical sciences and not religion was advanced over the ensuing ~2,400 years to the beginning of the industrial revolution.

Informed by advances in the industry, biologists and chemists in the late 19th century and early 20th century began to adopt analogous approaches and technologies to those successfully employed in physics, engineering, and industry. Over the first half of the 20th century, these approaches were parlayed into the successful identification of food-derived enzyme cofactors—vitamins; the biological basis of viral and bacterial infections and the means to intercept them—vaccines and antibiotics; and the successful identification of genetic material—DNA, and how genetic information encodes for proteins. These advances contributed to astonishing gains in the understanding of the biological sciences and medicine.

In the most general sense, the tools and reasoning that drove industrialization were then, and are now still, employed to solve biological problems. At the core of the reasoning is a form of reductionism that is instantiated in the scientific method in the generalized hypothesis of “what part is ascribed to what function.”

A precision approach to learning would be most predictive when there is a simple and orderly relationship between a part and its function. Such examples in non-biological systems might include a tire on a bicycle, or in a biological system, a gene and protein critical for energy synthesis and life itself. In these instances, the measurement of the tire or the gene would be expected to correlate with the function of the system. The precision biology approach has its greatest positive predictive value in non-biological systems that are designed and engineered by humans, as these systems, by definition, follow a 1:1 relationship between parts and function. The precision biology approach also has value in biological systems where there is a hypothesized and measurable relationship between a part and its function. However, the precision biology approach would lose its positive predictive value in biological systems when there is not a 1:1 relationship between a part and its function. The instance where there is no discernable relationship between a part and its function is the case where a part, or two or more parts, form a new structure or perform a new function not readily predictable or discernable from the part or parts alone. This property, the ability of one or more parts to form a structure or perform a function not resident in the/a part alone, is its “emergent structure or function” and collectively its “emergent property or properties.”

The current precision biology approach to understanding the function of biological and complex non-biological systems is limited by the inability to measure, quantify, predict, control, maximize, design, and engineer complex adaptive biological and non-biological systems based on their emergent properties. These limitations are observed at nearly all hierarchies of biological systems, including the biosphere itself.

The precision biology approach is limited in understanding biological and human function. The parts-based approach implicitly considers the biological system or human as a self-contained collection of parts from which function is to be sorted and calculated. It embodies a pre-vitamin paradigm and ironically is limited today by an incomplete inventory of the parts responsible for function. Until relatively recently, the precision biology approach in humans omitted ~1-10 trillion bacteria that form the human microbiome. The precision biology approach also discounts or omits in their entirety other parts and the context in which they exist. Examples would include food. While there are estimated to be over 30,000 plant-derived small molecules called phytonutrients in the human diet, the function of only <0.1% of these phytonutrients—the vitamins—are understood. Additionally, the precision biology approach discounts the criticality and importance of certain classes of enzymes and deprioritizes their study. This would include but is not limited to those in metabolism that interact with substances derived from the milieu exterior, such as oxidoreductase enzymes. When applied to medicine, the precision biology paradigm oversimplifies the complexity of biological systems. It is very limited in its ability to diagnose and develop treatments to disease when the function it seeks to understand is emergent in origin.

The precision biology approach is also limited in understanding the health of a species or collection of species and resources. The precision biology approach considers health as the null case or absence of disease obtained through a process of disease elimination. Today, human health is a concept, not an actuality. It should be noted that this was/is not always the case in many non-reductionist cultures. The concept of health as an energy state—essentially an emergent property—independent from disease is common to many eastern cultures and religions, chakras, reiki, prana, chi, can be traced to 400 BC, and more recently in the West as elan vital in the 1900s. Health is essentially the baseline of function of a biological system, and may also be referred to as homeostasis. But homeostasis is an emergent property: a complex interplay of many parts to produce interchangeable forms and functions that are not resident in or discernable by the precision biology approach. The precision biology approach significantly fails in an open system in which homeostasis (health) is dependent upon the complex interaction of the milieu interior and milieu exterior. As a result, disease measures are used to define the absence of health. Disease measures are very poor surrogates for absence of health in that they substantially lag changes in health or “health capacity”: the resilience (adaptivity) of a system expressed primarily by its ability to persist or achieve some core function.

The precision biology approach is also limited in “genetic engineering” and industrial biology. The absence of a 1:1 relationship between the change of a gene and the intended outcome frequently results in the generation of non-homeostatic (un-healthy) states inconsistent with the intended new (synthetic function) or viability.

The precision biology approach is also limited in terms of its implementation. It requires highly specialized and expensive equipment for measurement. The precision biology approach is limited in terms of learning rate. It results in an unmanageable number of false (positive) discoveries and discounts the possibility of unexpected outcomes, in other words, unintended consequences”. The cost, risk and time to learn are very high and increasing consistent with Eroom's Law. The precision biology approach is limited by its invasiveness and ethicality. Whereas emergent properties are often quantifiable from the exterior, the precision biology approach typically involves invasive tests or experimental euthanasia that can be unethical, injurious or lethal—all of which reduce the practicality of gaining frequent and sufficient measurements. Without large sample sizes and real human test data, it is extremely difficult to obtain the necessary statistical power to differentiate good hypotheses from bad. This exacerbates the problems of false discovery and further slows the learning rate of the biological sciences generally. The precision biology approach is limited by its silence on anthropocentric effects on biological function. It considers that DNA contains all the relevant biological information, and that function cascades forthwith. It does not consider other physical or biological information systems such as temperature, inter-/intra-species dependencies, gravity, magnetic fields, currents, populations, and the like all of which have undergone dramatic changes in the last 100 years of the “anthropocene.” The precision biology approach is also limited by the conjecturing of a “paradigm” that “DNA is the book of life” is “truth” despite evidence to the contrary. Despite these clear limitations to the precision biology approach/paradigm, there is dogmatic continuation of this approach as a series of “-omics” revolutions. Because there may be an infinite set of taxonomies of parts, the parts-based approach is essentially inexhaustible and therefore, non-falsifiable: there is no means by which the precision biology paradigm can prove itself wrong. This non-falsifiability has been made worse by modern machine learning tools. It is now conjectured that the knowledge gap in the precision biology approach is not the approach, but insufficiencies in analytical methods to understand the parts. While machine learning tools will certainly have utility for those problems solvable by the precision biology approach, more analysis and data gathering has never supplanted the need for new measurements that make visible what has been hidden. The precision biology approach simply does not measure or acknowledge emergent properties of biological systems by which the essence of life is defined.

Life exists and persists across a wide range of dynamic environmental conditions. Diverse forms of life can be found in extreme temperatures, pressures, pH conditions, and chemical solutions. Yet common to all living organisms is the emergent property of extracting energy from their environment and employing that energy in accordance with a metabolic strategy that “fits” their environment. While there are nearly as many such metabolic strategies as there are species, all must obey basic thermodynamic principles of heat transfer and therefore must be thermally appropriate for their environment. In other words, in order to persist, all must have a proper “heat fit” with their environment. Should a living organism lose the ability to maintain its “heat fit” within its environment, its metabolism will fail and it will no longer persist (i.e., it will die). This basic principle of heat fit governs all living things, from primitive bacteria to plants to organisms as complex as human beings. Therefore, effective maintenance or regulation of a living organism's heat fit depends on the efficient design and execution of its respective metabolic strategy in the face of dynamic changes in its environment. Because extracting replacement energy from the environment has a metabolic cost of its own, the aspects of the metabolic strategy that have the most potential for exhausted energy pose the most risk to the organism maintaining its heat fit.

What is needed, therefore, is a new way to measure, quantify, and interpret emergent properties of biological and non-biological systems to understand the function of complex adaptive systems, enabling prediction, optimization, design, and engineering of biological and non-biological systems. This way should be considered as part of a “consilience approach” that considers the totality of all biological and non-biological parts and systems and their emergent properties within.

New systems, devices and methods are needed to understand, with greater accuracy, accessibility, scalability and more readily, the function of complex adaptive systems. Such systems, devices and methods would, ideally, enable prediction, optimization, design, and engineering of biological and even non-biological systems, and would consider the totality of all biological and non-biological parts and systems.

### SUMMARY

The disclosure provides systems, devices, and methods for continuously and contextually characterizing an individual's metabolic state and/or heat fit by measuring their thermal signature to assess what is referred to as a thermoregulatory phenotype. Changes relative to this phenotype are sensitive indicators of change in health state. The systems, devices, and methods are designed and configured such that they deliver, in human use, general associations between an individual's thermal signature and physiologic reserve. Additional information is available within the thermal signature to an actionable assessment of health state.

In certain embodiments, a system configured to identify at least one recognizable feature associated with a health state of a subject, the system comprising: at least one sensor for measuring a heat flux of said subject and generating a data stream therefrom, wherein said data stream includes a plurality of heat flux measurements; and a processor configured to receive the data stream and recognize said at least one recognizable feature associated with said health state.

In certain embodiments, wherein the at least one sensor for measuring heat flux comprises at least one sensor array for simultaneously measuring heat elimination by said subject and an environmental temperature proximal to said subject.

In certain embodiments, wherein the plurality of heat flux measurements comprise a plurality of heat elimination measurements expressed as a function of environmental temperature.

In certain embodiments, the system further comprising at least one transition state corresponding to the at least one recognizable feature of the data stream.

In certain embodiments, wherein the at least one recognizable feature associated with a health state corresponds to a biological response.

In certain embodiments, wherein the at least one recognizable feature has a duration less than one hour.

In certain embodiments, wherein the at least one recognizable feature has a duration of one hour or more.

In certain embodiments, wherein the at least one recognizable feature corresponds to a homeostatic state of the subject.

In certain embodiments, wherein the data stream further comprises at least one work quantification associated with said plurality of heat flux measurements.

In certain embodiments, wherein the at least one work quantification corresponds to an amount of energy a user expends to enter at least one transition state corresponding to the at least one recognizable feature of the data stream.

In certain embodiments, wherein the at least one work quantification further corresponds to an amount of energy the subject expends between a first transition state and a second transition state.

In certain embodiments, wherein said at least one work quantification corresponds to a motion of said subject.

In certain embodiments, the system further configured to identify at least one motif, wherein the at least one motif comprises a plurality of heat flux measurements.

In certain embodiments, wherein there is at least one transition state between a first motif and a second motif.

In certain embodiments, the system further configured to identify at least one discord comprising a plurality of heat flux measurements that do not correspond to the at least one recognizable feature.

In certain embodiments, wherein one or more recognizable features is indicative of thermal regulation.

In certain embodiments, wherein the one or more recognizable features comprises one or more unique shapes, wherein the one or more unique shapes is at least one of: a thermal range; and a thermal trend.

In certain embodiments, wherein the one or more unique shapes are indicative of a health transition.

In certain embodiments, the system further configured to identify a difference between a first heat flux measurement of the plurality of heat flux measurements and a second heat flux measurement of the plurality of heat flux measurements.

In certain embodiments, wherein the at least one transition state is indicative of a change in homeostasis of a subject.

In certain embodiments, wherein the processor forecasts a plurality of future plurality of heat flux measurements based on the at least one recognizable feature identified.

In certain embodiments, wherein the processor forecasts a future recognizable feature based on the at least one recognizable feature identified.

In certain embodiments, wherein the processor determines the subject is an anomaly of a population based on the at least one recognizable feature.

In certain embodiments, wherein the population comprises one or more subjects with a health status substantially similar to a health status of the subject.

In certain embodiments, wherein the population comprises one or more subjects with a health transition substantially similar to a health statement of the subject.

In certain embodiments, wherein the processor generates a recommended counter action based on the at least one recognizable feature.

In certain embodiments, wherein the recommended counter action comprises at least one of: apply cold compress; rest; engage in physical activity; cease administration of a medicament; commence administration of a medicament; go inside; go outside; increase hydration; decrease hydration; and elevate one or more extremity.

In certain embodiments, wherein the processor is configured to analyze the data stream.

In certain embodiments, a method of identifying at least one recognizable feature associated with a biological response comprising: measuring heat flux of a subject; generating a data stream wherein said data stream includes a plurality of heat flux measurements; and recognizing said at least one recognizable feature from said data stream.

In certain embodiments, the method further comprising comparing analysis of the at least one recognizable feature identified for a first subject to the analysis of the at least one recognizable feature identified for a second subject.

In certain embodiments, a method of assessing resilience of thermoregulation in a subject and generating a recommendation to engage in an intervention as to the subject based on the assessed resilience of thermoregulation, the method comprising: generating a thermoregulation resilience score for the subject over a pre-determined time period, wherein the thermoregulation resilience score is based on a data stream comprising a plurality of thermoregulation measurements of the subject; determining whether a need for an intervention as to the subject is above a pre-determined threshold of urgency; and generating a recommendation to engage in the intervention as to subject.

In certain embodiments, wherein the pre-determined time period is at least two days.

In certain embodiments, wherein the pre-determined time period is at least one day.

In certain embodiments, wherein the pre-determined time period is approximately one day.

In certain embodiments, wherein the pre-determined time period is less than one day.

In certain embodiments, wherein the pre-determined time period is approximately 8 hours.

In certain embodiments, wherein the thermoregulation resilience score is of a homeostatic robustness of a thermoregulation system of the subject.

In certain embodiments, wherein the homeostatic robustness of the subject is based on the resilience of the thermoregulation system in a transitory phase.

In certain embodiments, wherein an ability of the subject to regulate the thermoregulation system of the subject is based on a health status of the subject.

In certain embodiments, wherein the transitory phase includes a plurality of thermoregulation measurements inconsistent with the plurality of thermoregulation measurements that make up a normal state of the thermoregulation system of the subject.

In certain embodiments, wherein the transitory phase persists for an extended period of time greater than a predetermined period of time.

In certain embodiments, the method further comprising alerting a medical practitioner that the recommendation has been generated.

In certain embodiments, wherein a non-extended period of time of the transitory phase indicates a resilient thermoregulation system of the subject.

In certain embodiments, wherein the extended period of time of the transitory phase indicates a non-resilient thermoregulation system of the subject.

In certain embodiments, wherein the thermoregulation resilience score generated is greater than a predetermined threshold.

In certain embodiments, wherein the thermoregulation resilience score generated is less than a predetermined threshold.

In certain embodiments, wherein the thermoregulation resilience score falls below a predetermined threshold for a predetermined pre-determined persistence period.

In certain embodiments, the method further comprising alerting a medical practitioner that a recommendation has been generated.

In certain embodiments, wherein the thermoregulation resilience score correlates with one or more interventions.

In certain embodiments, the method further comprising analyzing a variation in the plurality of thermoregulation measurements of the subject to determine a characterization of the variation.

In certain embodiments, wherein the plurality of thermoregulation measurements of the subject includes a plurality of heat flux measurements.

In certain embodiments, wherein the characterization of the variation is a pattern.

In certain embodiments, a method to identify a subject in need of urgent intervention, the method comprising: measuring a plurality of thermoregulation measurement of the subject; generating a data stream, over a time period, wherein the data stream includes a plurality of thermoregulation measurements; generating a score, for the time period, based on the data stream; identifying whether the subject is in urgent need of intervention; and sending an alert relating to the urgent need for intervention.

In certain embodiments, wherein the score is less than a predetermined score.

In certain embodiments, wherein the score indicates a thermoregulation system of the subject is non-resilient.

In certain embodiments, an alert system configured to generate an alert to intervene as to the well-being of a subject, the alert system comprising: at least one sensor for generating a plurality of thermoregulation measurements of the subject; a communication means for generating a data stream of thermoregulation measurements, wherein the data stream includes a plurality of thermoregulation measurements over a pre-determined time period; and a processor configured to receive the data stream and generate a score, associated with the pre-determined time period, based on the resilience of the thermoregulation measurement of the subject.

In certain embodiments, wherein the score is a resilient score when the score is greater than a predetermined score threshold.

In certain embodiments, the system further comprising an alarm configured to alert a user to intervene the subject when the score is less than a predetermined score threshold.

In certain embodiments, wherein the score is a non-resilient score that indicates the thermoregulatory system of the subject is non-resilient.

In certain embodiments, a method of identifying a subject in need of an intervention and alerting to another relating to the need for intervention, the method comprising: measuring a plurality of thermoregulation measurements of the subject; generating a data stream, over a time period, wherein the data stream includes a plurality of thermoregulation measurements; generating a score, for the time period, based on the data stream; grouping the score and a plurality of other biological measurements into a data set; identifying whether the subject is in need of intervention based at least in part on the score; and sending an alert relating to the need for intervention.

In certain embodiments, wherein the plurality of other biological measurements includes at least one of: heart rate; blood pressure; or skin temperature.

In certain embodiments, wherein identifying whether the need for intervention is urgent.

In certain embodiments, wherein the subject is in urgent need of intervention is based primarily on the score.

In certain embodiments, a method of identifying a patient having a health risk, the method comprising: measuring a plurality of thermoregulation measurement of the subject; generating a data stream, over a time period, wherein the data stream includes a plurality of thermoregulation measurements; generating a score, for the time period, based on the data stream; grouping the score and a plurality of other biological measurements into a data set; and identifying whether the subject has a health risk.

In certain embodiments, wherein identifying whether the subject has a health risk is used to determine at least one of: medical insurance risk of the subject; or heat stroke threshold of the subject.

### BRIEF DESCRIPTION OF THE DRAWINGS

FIG. 1A illustrates an example embodiment of a system detecting a recognizable feature.

FIG. 1B illustrates an example embodiment of a system detecting a recognizable feature.

FIG. 2A illustrates an example embodiment of a system detecting a recognizable feature.

FIG. 2B illustrates an example embodiment of a system detecting a recognizable feature.

FIG. 2C illustrates an example embodiment of a system detecting a recognizable feature.

FIG. 3A illustrates an example embodiment of a system detecting a recognizable feature.

FIG. 3B illustrates an example embodiment of a system detecting a recognizable feature.

FIG. 3C illustrates an example embodiment of a system detecting a recognizable feature.

FIG. 4A illustrates an example embodiment of a system detecting a recognizable feature.

FIG. 4B illustrates an example embodiment of a system detecting a recognizable feature.

FIG. 4C illustrates an example embodiment of a system detecting a recognizable feature.

FIG. 4D illustrates an example embodiment of a system detecting a recognizable feature.

FIG. 5A illustrates an example embodiment of a system detecting a recognizable feature.

FIG. 5B illustrates an example embodiment of a system detecting a recognizable feature.

FIG. 5C illustrates an example embodiment of a system detecting a recognizable feature.

FIG. 5D illustrates an example embodiment of a system detecting a recognizable feature.

FIG. 5E illustrates an example embodiment of a system detecting a recognizable feature.

FIG. 5F illustrates an example embodiment of a system detecting a recognizable feature.

FIG. 5G illustrates an example embodiment of a system detecting a recognizable feature.

FIG. 5H illustrates an example embodiment of a system detecting a recognizable feature.

FIG. 5I illustrates an example embodiment of a system detecting a recognizable feature.

FIG. 6A illustrates an example embodiment of a system detecting a recognizable feature.

FIG. 6B illustrates an example embodiment of a system detecting a recognizable feature.

FIG. 6C illustrates an example embodiment of a system detecting a recognizable feature.

FIG. 6D illustrates an example embodiment of a system detecting a recognizable feature.

FIG. 6E illustrates an example embodiment of a system detecting a recognizable feature.

FIG. 7A illustrates an example embodiment of a system detecting a recognizable feature.

FIG. 7B illustrates an example embodiment of a system detecting a recognizable feature.

FIG. 7C illustrates an example embodiment of a system detecting a recognizable feature.

FIG. 7D illustrates an example embodiment of a system detecting a recognizable feature.

FIG. 8A illustrates an example embodiment of a system detecting a recognizable feature.

FIG. 8B illustrates an example embodiment of a system detecting a recognizable feature.

FIG. 8C illustrates an example embodiment of a system detecting a recognizable feature.

FIG. 8D illustrates an example embodiment of a system detecting a recognizable feature.

FIG. 9A illustrates an example embodiment of a system detecting a recognizable feature.

FIG. 9B illustrates an example embodiment of a system detecting a recognizable feature.

FIG. 9C illustrates an example embodiment of a system detecting a recognizable feature.

FIG. 9D illustrates an example embodiment of a system detecting a recognizable feature.

FIG. 10A illustrates an example embodiment of a system detecting a recognizable feature.

FIG. 10B illustrates an example embodiment of a system detecting a recognizable feature.

FIG. 10C illustrates an example embodiment of a system detecting a recognizable feature.

FIG. 11A illustrates an example embodiment of a system detecting a recognizable feature.

FIG. 11B illustrates an example embodiment of a system detecting a recognizable feature.

FIG. 11C illustrates an example embodiment of a system detecting a recognizable feature.

FIG. 12A illustrates an example embodiment of a system detecting a recognizable feature.

FIG. 12B illustrates an example embodiment of a system detecting a recognizable feature.

FIG. 12C illustrates an example embodiment of a system detecting a recognizable feature.

FIG. 12D illustrates an example embodiment of a system detecting a recognizable feature.

FIG. 12E illustrates an example embodiment of a system detecting a recognizable feature.

FIG. 13A illustrates an example embodiment of a system detecting a recognizable feature.

FIG. 13B illustrates an example embodiment of a system detecting a recognizable feature.

FIG. 14A illustrates an example embodiment of a system detecting a recognizable feature.

FIG. 14B illustrates an example embodiment of a system detecting a recognizable feature.

FIG. 14C illustrates an example embodiment of a system detecting a recognizable feature.

FIG. 14D illustrates an example embodiment of a system detecting a recognizable feature.

FIG. 14E illustrates an example embodiment of a system detecting a recognizable feature.

FIG. 14F illustrates an example embodiment of a system detecting a recognizable feature.

FIG. 14G illustrates an example embodiment of a system detecting a recognizable feature.

FIG. 14H illustrates an example embodiment of a system detecting a recognizable feature.

FIG. 15A illustrates an example embodiment of a system detecting a recognizable feature.

FIG. 15B illustrates an example embodiment of a system detecting a recognizable feature.

FIG. 15C illustrates an example embodiment of a system detecting a recognizable feature.

FIG. 15D illustrates an example embodiment of a system detecting a recognizable feature.

FIG. 15E illustrates an example embodiment of a system detecting a recognizable feature.

FIG. 15F illustrates an example embodiment of a system detecting a recognizable feature.

FIG. 15G illustrates an example embodiment of a system detecting a recognizable feature.

FIG. 15H illustrates an example embodiment of a system detecting a recognizable feature.

FIG. 15I illustrates an example embodiment of a system detecting a recognizable feature.

FIG. 15J illustrates an example embodiment of a system detecting a recognizable feature.

FIG. 15K illustrates an example embodiment of a system detecting a recognizable feature.

FIG. 15L illustrates an example embodiment of a system detecting a recognizable feature.

FIG. 15M illustrates an example embodiment of a system detecting a recognizable feature.

FIG. 15N illustrates an example embodiment of a system detecting a recognizable feature.

FIG. 16A illustrates an example embodiment of a system detecting a recognizable feature.

FIG. 16B illustrates an example embodiment of a system detecting a recognizable feature.

FIG. 16C illustrates an example embodiment of a system detecting a recognizable feature.

FIG. 16D illustrates an example embodiment of a system detecting a recognizable feature.

FIG. 16E illustrates an example embodiment of a system detecting a recognizable feature.

FIG. 16F illustrates an example embodiment of a system detecting a recognizable feature.

FIG. 16G illustrates an example embodiment of a system detecting a recognizable feature.

FIG. 16H illustrates an example embodiment of a system detecting a recognizable feature.

FIG. 16I illustrates an example embodiment of a system detecting a recognizable feature.

FIG. 16J illustrates an example embodiment of a system detecting a recognizable feature.

FIG. 16K illustrates an example embodiment of a system detecting a recognizable feature.

FIG. 16L illustrates an example embodiment of a system detecting a recognizable feature.

FIG. 16M illustrates an example embodiment of a system detecting a recognizable feature.

FIG. 16N illustrates an example embodiment of a system detecting a recognizable feature.

FIG. 17A illustrates an example embodiment of a system detecting a recognizable feature.

FIG. 17B illustrates an example embodiment of a system detecting a recognizable feature.

FIG. 17C illustrates an example embodiment of a system detecting a recognizable feature.

FIG. 17D illustrates an example embodiment of a system detecting a recognizable feature.

FIG. 17E illustrates an example embodiment of a system detecting a recognizable feature.

FIG. 17F illustrates an example embodiment of a system detecting a recognizable feature.

FIG. 17G illustrates an example embodiment of a system detecting a recognizable feature.

FIG. 17H illustrates an example embodiment of a system detecting a recognizable feature.

FIG. 17I illustrates an example embodiment of a system detecting a recognizable feature.

FIG. 17J illustrates an example embodiment of a system detecting a recognizable feature.

FIG. 17K illustrates an example embodiment of a system detecting a recognizable feature.

FIG. 17L illustrates an example embodiment of a system detecting a recognizable feature.

FIG. 17M illustrates an example embodiment of a system detecting a recognizable feature.

FIG. 17N illustrates an example embodiment of a system detecting a recognizable feature.

FIG. 18A illustrates an example embodiment of a system detecting a recognizable feature.

FIG. 18B illustrates an example embodiment of a system detecting a recognizable feature.

FIG. 18C illustrates an example embodiment of a system detecting a recognizable feature.

FIG. 18D illustrates an example embodiment of a system detecting a recognizable feature.

FIG. 18E illustrates an example embodiment of a system detecting a recognizable feature.

FIG. 18F illustrates an example embodiment of a system detecting a recognizable feature.

FIG. 18G illustrates an example embodiment of a system detecting a recognizable feature.

FIG. 18H illustrates an example embodiment of a system detecting a recognizable feature.

FIG. 18I illustrates an example embodiment of a system detecting a recognizable feature.

FIG. 18J illustrates an example embodiment of a system detecting a recognizable feature.

FIG. 18K illustrates an example embodiment of a system detecting a recognizable feature.

FIG. 18L illustrates an example embodiment of a system detecting a recognizable feature.

FIG. 18M illustrates an example embodiment of a system detecting a recognizable feature.

FIG. 18N illustrates an example embodiment of a system detecting a recognizable feature.

FIG. 19A illustrates an example embodiment of a system detecting a recognizable feature.

FIG. 19B illustrates an example embodiment of a system detecting a recognizable feature.

FIG. 19C illustrates an example embodiment of a system detecting a recognizable feature.

FIG. 19D illustrates an example embodiment of a system detecting a recognizable feature.

FIG. 19E illustrates an example embodiment of a system detecting a recognizable feature.

FIG. 19F illustrates an example embodiment of a system detecting a recognizable feature.

FIG. 19G illustrates an example embodiment of a system detecting a recognizable feature.

FIG. 19H illustrates an example embodiment of a system detecting a recognizable feature.

FIG. 19I illustrates an example embodiment of a system detecting a recognizable feature.

FIG. 19J illustrates an example embodiment of a system detecting a recognizable feature.

FIG. 19K illustrates an example embodiment of a system detecting a recognizable feature.

FIG. 19L illustrates an example embodiment of a system detecting a recognizable feature.

FIG. 19M illustrates an example embodiment of a system detecting a recognizable feature.

FIG. 19N illustrates an example embodiment of a system detecting a recognizable feature.

FIG. 20A illustrates an example embodiment of a system detecting a recognizable feature.

FIG. 20B illustrates an example embodiment of a system detecting a recognizable feature.

FIG. 20C illustrates an example embodiment of a system detecting a recognizable feature.

FIG. 20D illustrates an example embodiment of a system detecting a recognizable feature.

FIG. 20E illustrates an example embodiment of a system detecting a recognizable feature.

FIG. 20F illustrates an example embodiment of a system detecting a recognizable feature.

FIG. 20G illustrates an example embodiment of a system detecting a recognizable feature.

FIG. 20H illustrates an example embodiment of a system detecting a recognizable feature.

FIG. 20I illustrates an example embodiment of a system detecting a recognizable feature.

FIG. 20J illustrates an example embodiment of a system detecting a recognizable feature.

FIG. 20K illustrates an example embodiment of a system detecting a recognizable feature.

FIG. 20L illustrates an example embodiment of a system detecting a recognizable feature.

FIG. 20M illustrates an example embodiment of a system detecting a recognizable feature.

FIG. 20N illustrates an example embodiment of a system detecting a recognizable feature.

FIGS. 21A and 21B illustrate an example embodiment of plurality of thermoregulation measurements of a subject over a period of time, wherein the data depicted in FIG. 21A is unprocessed and the data depicted in FIG. 21B is represented as space-filling hexagons to facilitate point-to-point distance computation.

FIGS. 22A, 22B, 22C, 22D, 22E, 22F, 22G, 22H, 221, 22J, 22K, 22L, 22M, 22N, 22O illustrate an example of embodiments of binned pluralities of thermoregulation measurements of a subject over a period of fifteen days.

FIG. 23 illustrates an example embodiment of a plurality of thermoregulation resilience scores of a subject over a period of time defined as one month.

FIG. 24 illustrates an example embodiment of a plurality of thermoregulation resilience scores of a subject over a period of time defined as one month.

FIG. 25 illustrates an example embodiment of a plurality of thermoregulation resilience scores of a subject over a period of time defined as one month.

FIG. 26 illustrates an example embodiment of a plurality of thermoregulation resilience scores of a subject over a period of time defined as one month.

FIG. 27 illustrates an example embodiment of a plurality of thermoregulation measurements of three subjects over a period of time defined as one day.

FIG. 28 illustrates an example embodiment of a plurality of thermoregulation resilience scores of a plurality of individuals plotted as a function of a combination of a plurality of thermoregulation resilience scores and a plurality of other biological measurements.

FIG. 29 illustrates an example embodiment of a plurality of thermoregulation resilience scores of a plurality of individuals plotted as a function of a combination of a plurality of thermoregulation resilience scores and a plurality of other biological measurements.

### DETAILED DESCRIPTION OF THE PREFERRED EMBODIMENT

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part incorporated herein by reference in their entireties for the purposes indicated by the context of their citation herein. However, the citation of any document is not to be construed as an admission that it is prior art with respect to the present disclosure.

The generation, analysis, and use of data relating to the health capacity of biological systems has been explored. More specifically, the use of sensors, and combinations of sensors, for capturing data related to the health capacity of biological systems has also been explored. Health capacity can refer to the resilience (adaptivity) of a system expressed primarily by its ability to persist or achieve some core function. To assess the health capacity of a system, the one may interpret the emergent factors of the system.

The Scholander-Irving model depicts a pattern of changes of resting metabolic of an endothermic homeotherm over a range of ambient temperatures. Per Scholander and Laurence Irving were interested researching how warm-blooded birds and mammals maintain body temperature. With such interest, they discovered warm-blooded birds and mammals maintain body temperature by balancing their rate of metabolic heat production and the rate of heat lost to the environment.

However, in relation to humans, one may have limited knowledge regarding their health capabilities, health status, disease state, general state of health, etc. One may gain limited insight into these areas of knowledge only with sophisticated physiologic measurement tools, to which few may have access.

### Definitions

“Analysis,” as used herein, refers to any description of characteristics or features of any observed sequence of information. Types of analysis include, but are not limited to, analyzing raw measurements such as wherein the numerical values of raw measurements can be used directly as features; resampled measurements such as wherein a set of raw measurements are grouped by and represented by the mean value of the group or some other group statistic; distributional representation of measurements such as wherein the distribution of a collection of measurements may be represented in regularly spaced bins or irregular bins which have been determined by some other process such as a Gaussian Mixture model; statistical tests over sets of measurements such as the Hartigan DIP test of multimodality or Stationarity tests which can be used to detect changes over time; fit parameters from physical models such as a 3-compartment model of body heat content, and extensions thereof or fitting hemodynamic parameters of human circulatory system; parameters of universal mathematical models such as those which cannot be reduced to any simple parameter of a physical model and which may include non-physical control parameters which summarize structure in the dataset, including, for example, Bifurcation parameter of conjugate logistic map and Eigenvalues of a hessian matrix of a “sloppy” physical model fit.

“Biological system,” as used herein, refers to any network of biologically relevant entities. In its broadest aspect, a biological system is any network of chemical reactions which exists as a persistent non-equilibrium configuration by its own devices. Biological systems encompass and span differing scales and are determined based different structures depending on the nature of the biological system. Examples of a biological system on a large scale include, for example, a population of microscopic organisms, a homogenous population of similar organisms living in proximity to one another (for example, a cell culture or a community of humans), a heterogeneous population of organisms living in a single ecosystem, biological networks. Examples of biological systems on a smaller scale include an individual organism, for example a single mammal such as a human, an organ or tissue system within such an organism, cellular organelle systems, or artificial life systems.

“Data stream,” as used herein, refers to a sequence of digitally encoded coherent signals (packets of data or data packets) used to transmit or receive information that is in the process of being transmitted. A data stream may be a set of extracted information from a data provider, and may comprise, for example, a sequence of ordered lists of elements (representing different signal components) and an associated sequence of timestamps.

“Disease,” as used herein, broadly refers to any condition that causes pain, dysfunction, distress, or death to the person afflicted. Thus, disease may include one or more injuries, disabilities, disorders, syndromes, infections, isolated symptoms, deviant behaviors, and atypical variations of structure and function. Diseases may affect biological organisms not only physically, but also mentally. Thus, in the case of a human afflicted with a disease, contracting and living with a disease can alter the affected person's perspective on life. Examples of diseases include those identified and classified on the World Health Organization's 10th revision of the International Statistical Classification of Diseases and Related Health Problems (ICD-10). Such diseases that may affect humans include, infectious and parasitic diseases, neoplasms, diseases of the blood and blood-forming organs, disorders involving the immune mechanism, endocrine diseases, nutritional diseases, metabolic diseases, mental and behavioral disorders, diseases of the nervous system, diseases of the eye and adnexa, diseases of the ear and mastoid process, diseases of the circulatory system, diseases of the respiratory system, diseases of the digestive system, diseases of the skin and subcutaneous tissue, diseases of the musculoskeletal system and connective tissue, diseases of the genitourinary system, diseases associated with pregnancy, childbirth and the puerperium, diseases originating in the perinatal period, congenital malformations, deformations and chromosomal abnormalities, as well as injuries, poisoning, and consequences of external causes.

“Discords,” as used herein, refers to any unusual or anomalous subsequences within a time series.

“Energy expenditure,” as used herein, in its most general sense, relates to the measurement of parameters that reflect heat or work in a biological system. “Energy expenditure” also refers to an entropy producing (irreversible) outlay of free energy to power an adaptive task within a biological system. An energy expenditure is largely irreversible (entropy-producing) so it represents energy which cannot be retrieved for other tasks. “Energy homeostasis” or “homeostatic control of energy balance,” as used herein, refers to a biological process that involves the coordinated homeostatic regulation of food intake (energy inflow) and energy expenditure (energy outflow).

“Emergent factor” or “emergent property” as used herein, refer to properties of a system not found in a part, or readily deducible from a detailed inventory and analysis of the parts contained within a system. They may be revealed by events, deviations from norm or other time dependent features in some measurable parameter of the system. Emergent properties may be observed directly or indirectly. Examples of emergent properties include amphotericity, conductivity, solvation capacity, ion mobility, oxidation-reduction potential, ligand association, hydration, electrolysis, thermal conductivity, heat capacity, thermal absorptivity, adhesion, cohesion, transparency, turbidity, incompressibility, polarity, dipolarity, dipole moment, diamagnetism, voltage range of the liquid phase, temperature range of the liquid phase, abundancy, and speciation, flux of energy, momentum, particles or other substances. Emergent factors also include thermoregulation and heat elimination, either as an absolute, static value of heat elimination or as a periodic function, for example a circadian periodicity of heat elimination.

“Feature,” as used herein, refers to any descriptive aspect, characteristic, attribute, quality, trait or property of a sequence, sub-sequence or datum of information. Examples of features include, but are not limited to, Chaotic, Repeating, Predictable, Spirals, Meanders, Rotations, Orbits, Dense/Diffuse, Symmetric/Asymmetric, Regular/Irregular/Intermittent, Periodic/Aperiodic, Cyclical, Similar/Dissimilar, Dynamic/Static, Rate of change, Direction of change, Inflections, Sequential, State Transitions, Anomalies/Outliers, Interruptions/Breaks, Oscillating/Persistent, Damped/Undamped, Increasing/Decreasing, Improving/Declining, Intersecting/Non-intersecting, Linear/Non-linear, Homogenous/Diverse, Monotonic/Polytonic, Serial/Out of order, Balanced/Imbalanced, Long/Short, Range, Modes/Medians/Averages/Standard Deviations, Equilibrium/Non-equilibrium, Stable/Unstable, Controlled/Uncontrolled, Homeomorphic/Isomorphic, Monomodal/Multimodal, Bounded/Unbounded, Trending, Exceeding/Not Exceeding a Threshold, Rising/Falling, Growing/Shrinking, Speeding up/Slowing down, Constant//Fluctuating, Peak/Valley, Zenith/Nadir, Asymptotic, Sudden, Steep/Gradual, Logistical/Polynomial mapping, Discrete/Continuous/Spaced/Discontinuous, Proportional, Gains/Losses, Energetic/Active/Inactive, Complexity/Simplicity, Attractors/Repellers, States, State transitions, Curves, Manifolds, Trajectories, Inflation/Deflation, Fractals, Momentum, Iterations, Dissipating/Developing, Presence/Absence, Perturbations, Variations/Invariation, Limited/Unlimited, Converging/Diverging, Velocity, Derivatives/Integrals, Exponential, Minimum/Maximum, Forward/Reverse/Inverse, Pulsing, Coalescing, Saturated/Unsaturated, Gain, Gradients, Deep/Shallow, Bifurcations/Splits, Points/Lines/Surfaces, Early/Late, and Distributed/Grouped.

“Forecasting,” as used herein, refers to any prediction of future data points within a time series based on what comes before the future data points.

“Health,” as used herein, refers to the baseline of function of a biological system, and may also be referred to as homeostasis. Health is not merely the absence of disease because health is an affirmative state independent of disease. Health is related to the ability of a biological system or organism to successfully adapt to a variety of challenges without significant loss of function. Physiologists, for example, may describe health as the sufficiency of a form of stored energy they call physiologic reserve—the ability of a human to positively respond to a stress. Physicists, as another example, may describe health as a capacity to incorporate, transform, and dissipate energy to persist. Cell biologists, as another example, may describe health as the baseline state of homeostasis—the ability of a cell or tissue to auto-regulate. Biochemists, as another example, may describe health as the control of anabolic and catabolic reactions in a metabolic network critical to biological function.

“Health capacity,” as used herein, is the resilience (adaptivity) of a system expressed primarily by its ability to persist or achieve some core function. The adjectives associated with high or low health capacity are ‘fit’ and ‘frail,’ respectively. Low health capacity—“Frailty”—increases the risk of disease or injury and the ability to withstand external stresses. Disease diminishes function such that health capacity may be diminished as a result. High health capacity “Fitness”—decreases the risk of injury and increases the ability to perform and to withstand external stresses. Early interception of disease can preserve and maintain health capacity and careful management of health capacity can prevent disease. Health capacity may be considered a correlation of a state, or energy budget, with a function of a biological system that defines the ability of the biological system to persist. Health capacity may be comprised of several quantities which may not necessarily be compared in the same manner or reduced to a single score. Data analytics may be used to discover the relationship between raw measurements and an abstract health capacity score. Dimensionality reduction or machine learning methods may be used to learn health capacity scores based on the raw measurement time series data and to predict adaptation and health outcomes.

“Health capacity rules,” as used herein, refers to the minimal set of attributes of an “energy budget” required to confer “health capacity.”

“Homeostasis,” as used herein, refers to processes and mechanisms for the regulation of the internal environment of a biological system, generally to limit variability of a state and/or maintain the status of a state. An example of a homeostasis mechanism at the organismal level is sweating, which serves to reduce temperature. An example of a homeostasis mechanism at the biochemical and cellular level is redox control and its regulation of metabolism.

“Infection,” as used herein, refers to an invasion of a biological system, typically of an organism, by one or more agents (or pathogens) that are not generally associated with the biological system. The agent is often a disease-causing agent. Infection also includes the propagation and multiplication of the agent, and the reaction of host biological system or organism. Infection also includes the generation of toxins, by or as a proximal cause of, the agent. Infectious disease, sometime referred to as “transmissible disease” or “communicable disease,” is a disease state resulting from an infection. Pathogens include, but are not limited to, viruses and related agents such as viroids and prions, bacteria, fungi which may be further classified, for example, as Ascomycota, including yeasts such as Candida, filamentous fungi such as Aspergillus, Pneumocystis species, and dermatophytes, Basidiomycota, including the human-pathogenic genus Cryptococcus, parasites which may be further classified, for example, as unicellular organisms (including, for example, malaria, Toxoplasma, Babesia), Macroparasites (including worms or helminths) such as nematodes such as parasitic roundworms and pinworms, tapeworms (cestodes), and flukes (trematodes, such as schistosomiasis), arthropods such as ticks, mites, fleas, and lice, can also cause human disease, which conceptually are similar to infections. Invasion of an animal body, such as a human body, by macroparasites may also be termed infestation but is consider, as used herein, to be a form of infection.

“Inflammation,” as used herein, refers to a particular, generic set of biological responses of body tissues to stimuli, such as pathogens, damaged cells, or irritants. Inflammation (and the associated condition, pre-inflammation) is a response involving immune cells, blood vessels, and molecular mediators that, at least in part, serves to eliminate the initial cause of cell injury, clear out necrotic tissues damaged from the original insult and initiate tissue repair. Signs of inflammation include increased heat, pain, redness, swelling, and loss of function. Inflammation may be considered a mechanism of innate immunity, as compared to adaptive immunity, which would be specific to a particular pathogen. Inflammation may be classified as acute or chronic. Acute inflammation is the initial response of the body to a stimuli and may be achieved by the increased movement of plasma and leukocytes (especially granulocytes) from the blood into the injured tissues. A series of biochemical events propagates and matures the inflammatory response, involving the local vascular system, the immune system, and various cells within the injured tissue. Chronic inflammation, often termed prolonged inflammation, may cause a progressive shift in the type of cells present at the site of inflammation, such as mononuclear cells, and is characterized by simultaneous destruction and healing of tissue.

“Metabolism,” as used herein, refers to transformation of energy by converting chemicals and energy into cellular components (anabolism) and decomposing organic matter (catabolismo. Living things require energy to maintain internal organization (homeostasis) and to produce the other phenomena associated with life.

“Modeling,” as used herein, refers to any descriptive representation or understanding of the processes or operation of biological systems which approximate such real world processes or operations.

“Motifs,” as used herein, refers to any repeated subsequences of information.

“Novelets,” as used herein, refers to any emerging motif which is both new and growing relative to the historical distribution of a sequence of information.

“Oncogenesis,” as used herein, refers to the formation of a cancer, whereby normal cells are transformed into cancer cells, also termed “tumorigenesis” or “carcinogenesis”. The process is characterized by changes at the cellular, genetic, and epigenetic levels and abnormal cell division. Mutations in DNA and epimutations disrupt processes involved in the programming and regulation of the normal balance between proliferation and programmed cell death.

“Response (to stimuli),” as used herein, refers to an action or modification in a biological system that results from external stimulus. A response may take any of several forms. For example, in the case of a unicellular organism, it may be the contraction resulting from exposure to the presence of chemicals in the environment. As another example, response may be a complex set of reactions involving all the senses of multicellular organisms. A response is often expressed by motion; for example, the leaves of a plant turning toward the sun (phototropism), and chemotaxis.

“Shapelets,” as used herein, refers to any small subsequences in a sequence of information which may be indicative of the state of a system.

“Snippets,” as used herein, refers to any subsequences which are representative of the sequence of information.

### General Criteria for Identifying and Selecting Emergent Factors

Emergent factors or emergent properties are identified and selected, for measurement, based on various criteria. Generally speaking, emergent factors that may be directly measured are preferred over emergent factors that may only be measured indirectly. Techniques used to measure (directly or indirectly) that are less invasive are preferred over techniques that are more invasive. Measurements that are reliable and associated with a single emergent factor, rather than multiple emergent factors, are preferred.

### General Example Embodiments

The technology disclosed in the present disclosure permits insights into the unique signature of one or more living systems, or a population of living systems, and thus into the health capacity of such a living system. The technology permits, for example, the measurement of the emergent property of heat elimination, measured either or both as an absolute value or as a periodic value. Absolute values of heat elimination may be indicative, or reflective, of energy expenditure. Absolute values of heat elimination may be indicative, or reflective, of metabolic rate insight. The periodicity of heat elimination may provide further insight into health capacity. The periodicity of heat elimination may be of a circadian periodicity (that is, on a cycle of from about 23 hour to a cycle of about 25 hour cycle), may be of a cycle shorter than circadian periodicity (for example, on a cycle of about 12, about 14, about 16, about 18, or about 20 hours), or may be longer than circadian (for example, every 2, 3, 4, 5, or 6 days, every week, or on a roughly monthly or 28-day, lunar, or annual cycle). Any such periodicity of heat elimination may serve as, or be reflective of, a unique signature a living system having a standard, acceptable health capacity. Deviations from that standard, acceptable periodicity of heat capacity may indicate, or otherwise reflect, sub-standard or unacceptable health capacity.

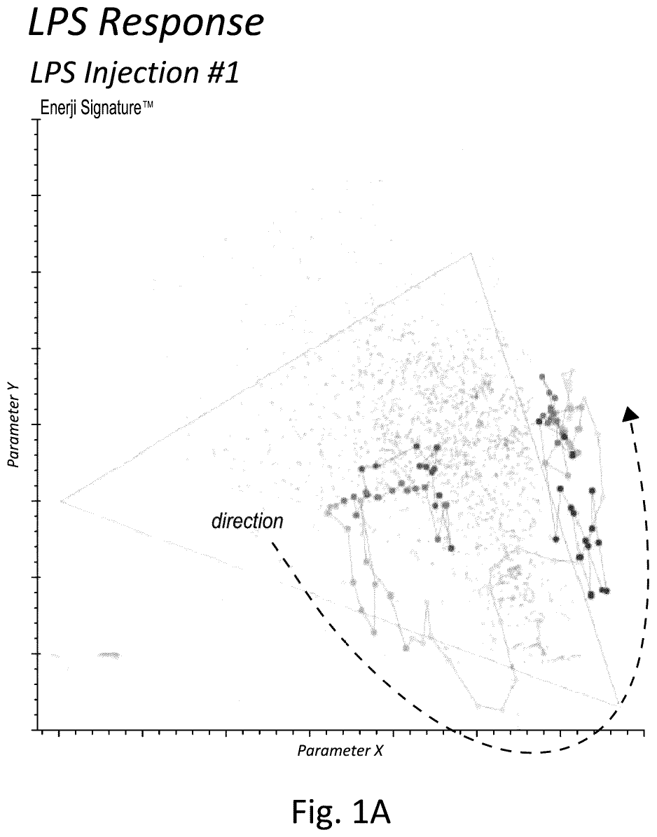

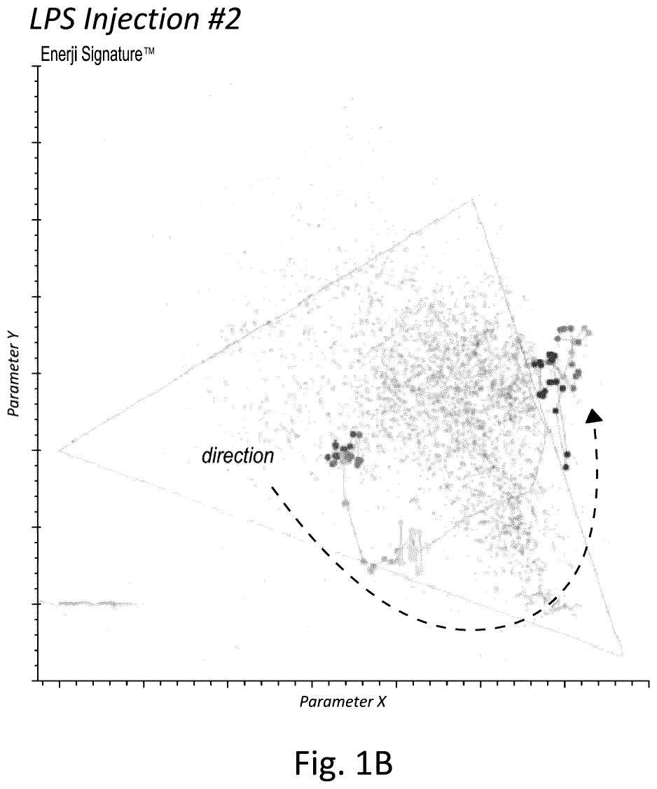

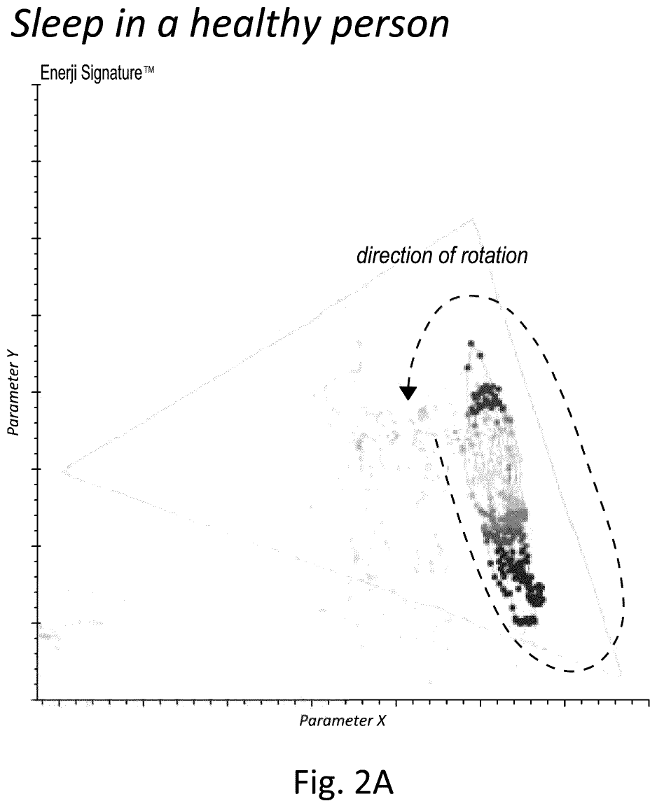

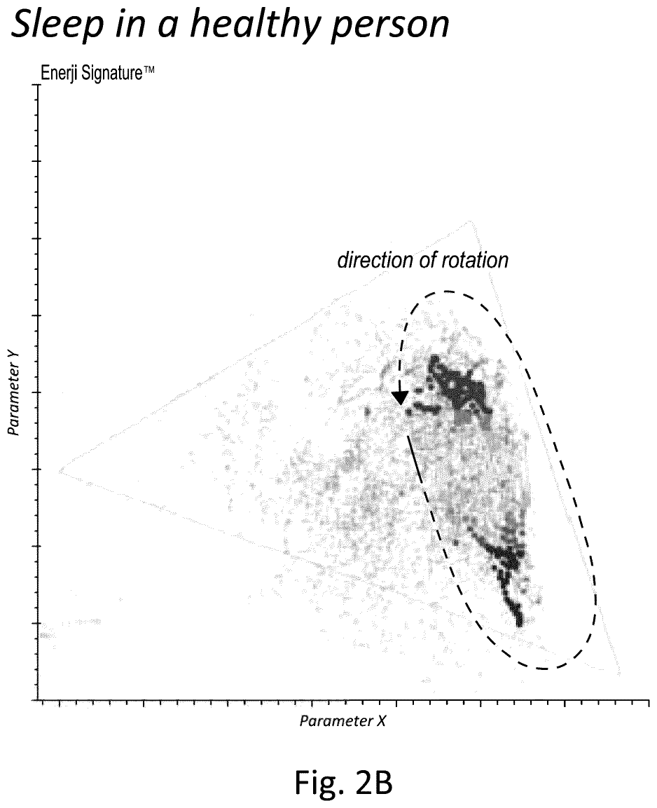

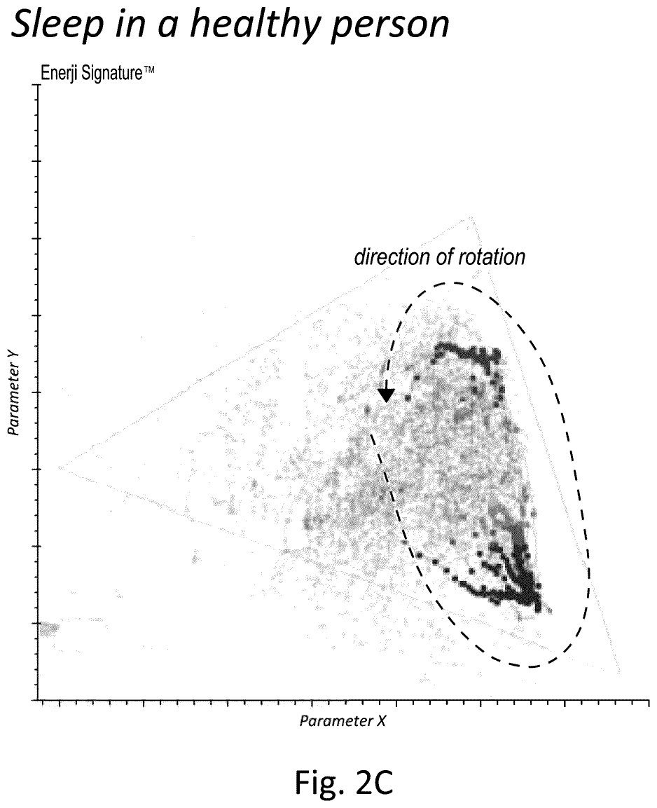

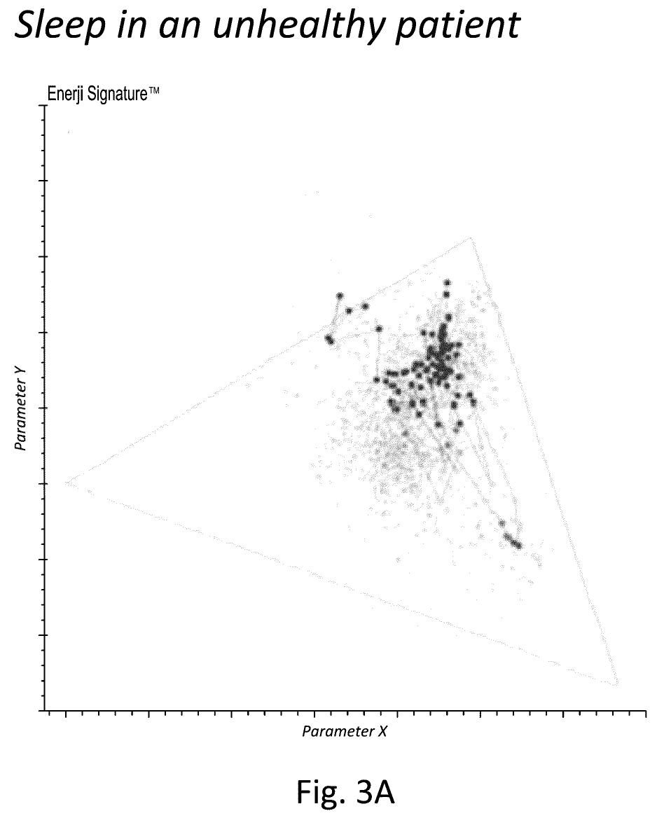

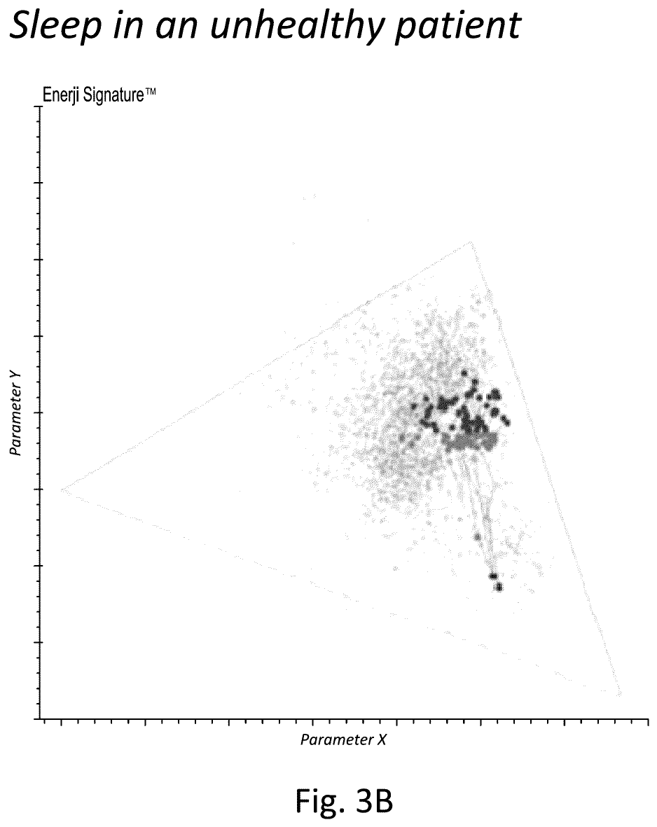

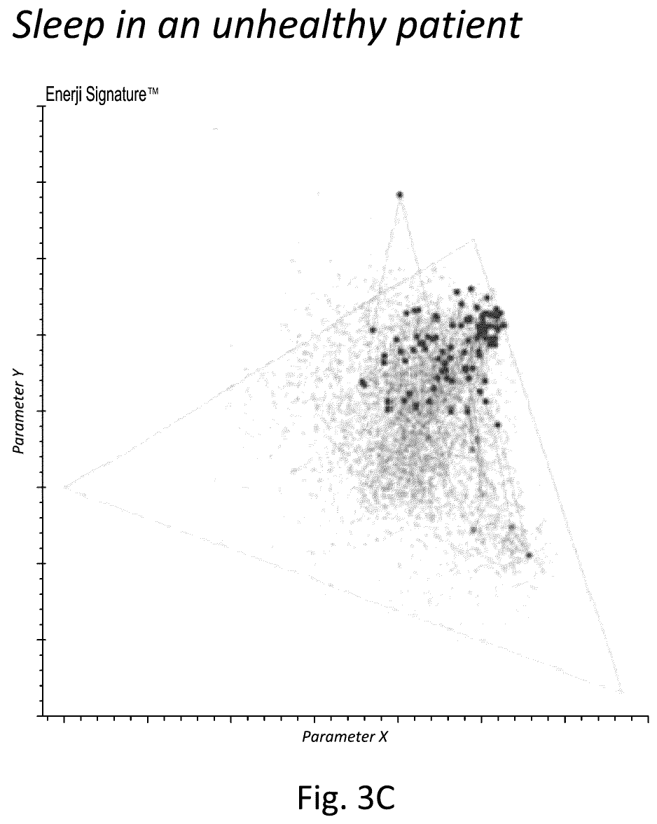

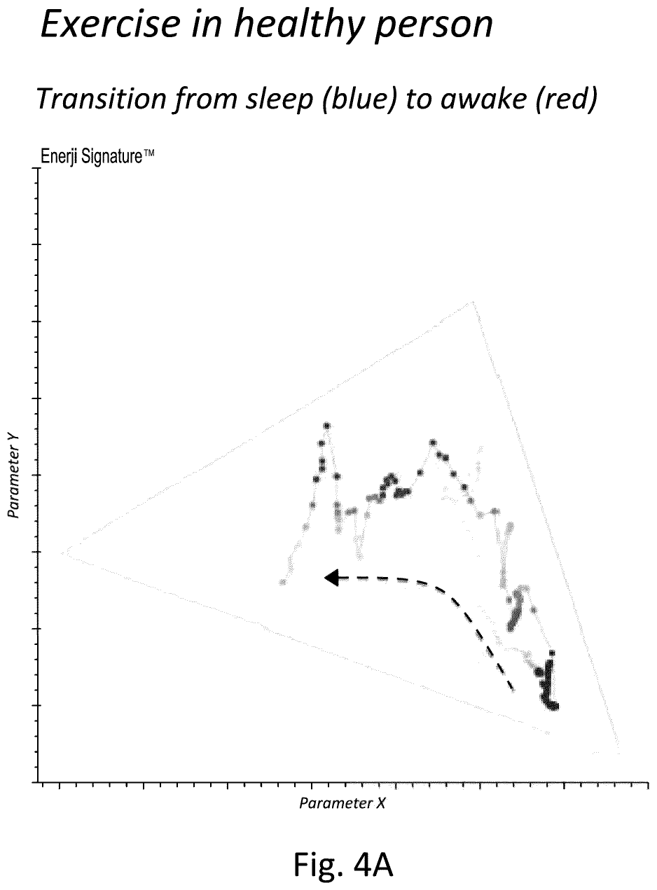

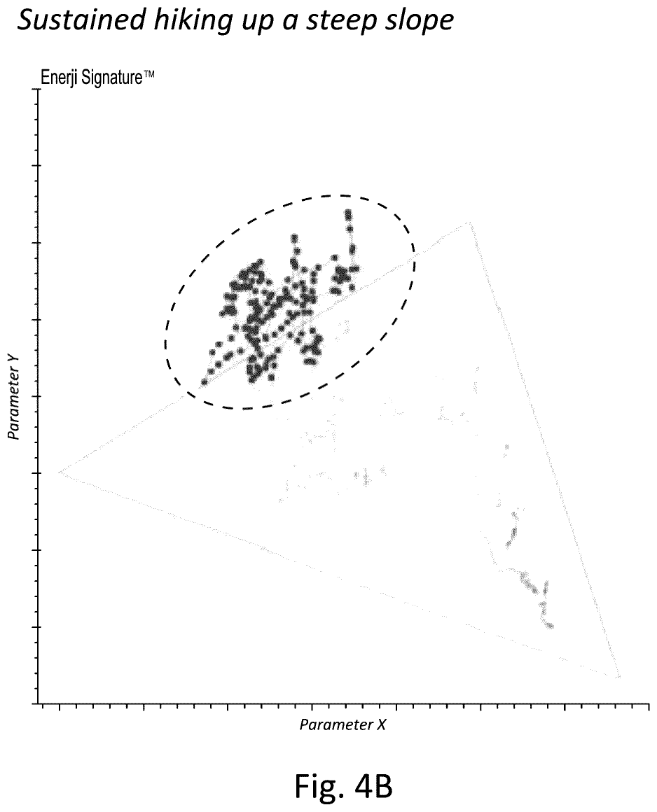

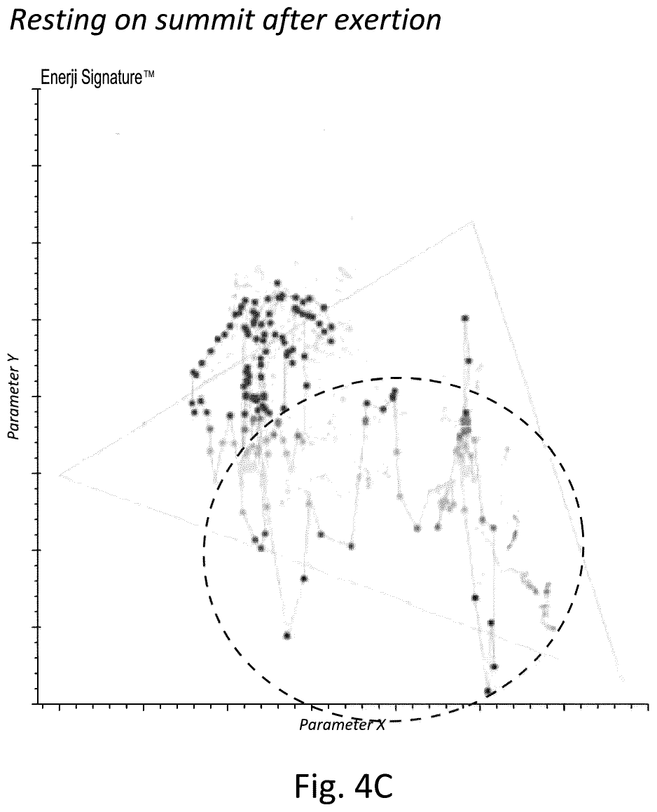

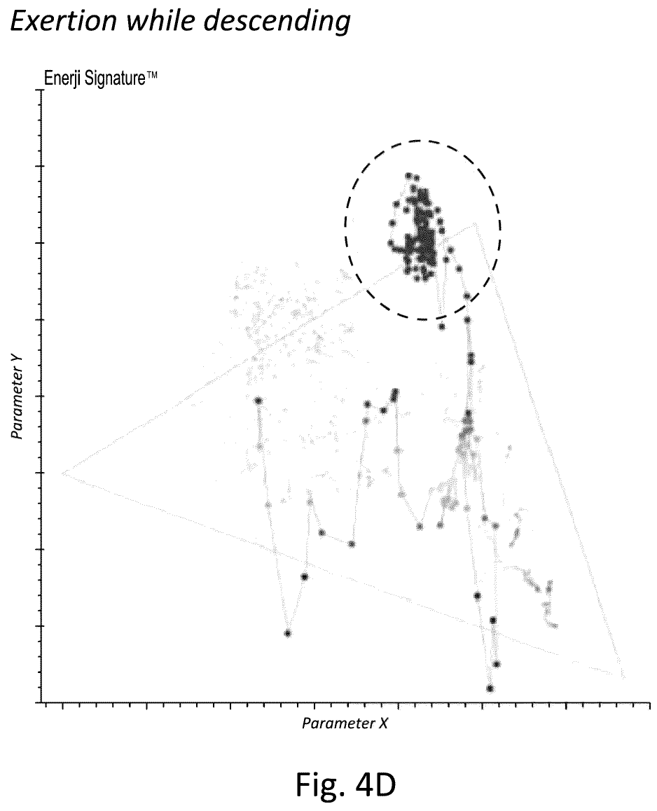

With respect to health capacity, “pre-symptomatic disease,” as used herein, refers to a process wherein health capacity is depleted by some metabolic task which is compensating of an environmental stress, the end result of which is disease and loss of function. This reduced health capacity is detectable as an anomalous energy expenditure associated with compensation or as some more general anomalous energy signature. Such a state of low or lowered health capacity places an individual at higher risk for loss of function generally, not merely from the initial stress or, more specifically, in a state of general susceptibility to disease or infection. Further, “symptomatic disease,” as used herein, refers to a state of impaired function or, more specifically, a disease state or a state in which an organism is, for example, infected by a viral pathogen. Current methodology, exemplified by the precision medicine paradigm, employs symptoms as indicators of disease (as illustrated in FIGS. 1 and 2). Aspects of the presently disclosed invention provide systems, devices, and methods for quantifying more fulsome metrics of health, exemplified by the measurement and assessment of “health capacity,” as either a static or dynamic measurement, to enable assessment of and increases in health capacity, and to identify pre-symptomatic changes in health capacity to permit early detection of disease or, more specifically, for example, infection (as illustrated in, for example FIGS. 2 and 3). An embodiment describing a learning strategy using measurement of energy and annotations to learn rules of health capacity is illustrated in FIG. 4.

Health, in one embodiment, is related to the ability of a biological system or organism to successfully adapt to a variety of challenges without significant loss of function. Physiologists, for example, may describe health as the sufficiency of a form of stored energy they call physiologic reserve—the ability of a human to positively respond to a stress. Physicists, as another example, may describe health as a capacity to incorporate, transform, and dissipate energy to persist. Cell biologists, as another example, may describe health as the baseline state of homeostasis—the ability of a cell or tissue to auto-regulate. Biochemists, as another example, may describe health as the control of anabolic and catabolic reactions in a metabolic network critical to biological function.

Each of these above-described perspectives or understandings of health is appropriate, in its context, be that the perspective of the physiologist, physicist, cell biologist, or biochemist, and contributes to the application of the disclosed technology. More specifically, the disclosed technology reconciles the above perspectives and understandings, and applies a novel conceptualization of health as high health capacity simultaneous with absence of disease—functional and fit. In one embodiment, health capacity reveals how health may be quantified by measuring physical properties of homeostasis. That is, health is quantifiable as the health capacity of a biological system. Other aspects of the disclosed technology relate to developing techniques that accurately measure an array of metrics of the state of the biological system, including, for example, the health the biological system. These techniques obtain and process information on an actionable time scale, at sufficiently high resolution.

Other aspects of the disclosed technology include: detecting features of emergent properties of biological systems; automating new recognitions and detections of emergent properties of biological systems to improve life; action plans to acquire, maintain, and promote health; allowing for faster learning and interpreting of complex biology from new health accuracy data streams; allowing frictionless collection, analysis, and decision support of health in subjects; developing a scalable solution to disease interception; reducing false-positives in diagnostics; finding causes of diseases; preventing further development of disease; early on set diagnosis of diseases; and explaining the development of symptoms. In some embodiments of the disclosed technology, properties of living systems and their physical-chemical properties are measured; these properties capture the emergent properties of living systems. In other embodiments, the disclosed technology is focused on emergent properties that gave rise to the boundary between physical and biological systems. The disclosed technology is thus applicable to synthetic biology and artificial biological systems, for example, protocells or industrial biology systems.

In another aspect, the disclosed technology relates to methods for detecting recognizable features or patterns from an array of health metrics from a subject. In some embodiments, the methods are integrated into an internet of things (IoT) architecture and infrastructure. In some embodiments, the methods detect features or patterns of metabolism at the scale of cells. These measurements of which the features may arise from may be intermitted, regular, or continuous. In some embodiments, the methods further comprise streaming arrays of such metrics into the Cloud, applying machine learning to “quantifying metabolism” and detecting changes before symptoms of diseases occur. In some embodiments, the methods non-invasively measure discrete physical parameters of biological systems at resolution and frequency that in real-time measure cellular physiology and homeostasis. In some embodiments, the methods define and quantify a quantity known as the health capacity of a subject, relating to the resilience of the subject's homeostasis, based on measured physical properties, energy and information of the subject.

The disclosed technology provides actionable information permitting the improvement of health capacity in a biological system. In preferred embodiment, individuals gain access to insight, not previously accessible, of their own health. Such actionability permits the observations of responses to particular stimuli in a specific biological system and permits alternation or influence of future stimuli. For example, an individual or health care professions may use the disclosed technology to alter the health of a biological system, for example, a patient or the individual himself or herself. It also permits a better understanding of the most influential determinants of health, for example, sleep patterns and duration, nutrition (including diet, supplements, medications, etc.), exercise (neuromuscular inputs, type, duration, periodicity, etc.) and other lifestyle decisions or detractors (e.g., pollution) that impact health. In a preferred embodiment, the disclosed technology permits an individual to be an efficient agent of his own or her own health. It also permits a better ability to plane or prepare for an action to be taken based on the status of the individual's health.

The disclosed technology provides an ability to compare detected recognizable features of an individual to detected recognizable features of a greater population of a similar to different recognizable feature.

The disclosed technology provides automated and automatable methods, thus addressing a major sources of healthcare inefficiency, manual testing and interpretation. Current healthcare testing and interpreting is generally centralized, and the interpretation of such testing is generally performed manually by healthcare professions. Both of these tasks are expensive and inefficient. The disclosed technology permits the digitalization of health data and interpretation associated with health capacity. Such digitization affords the ability to apply all the benefits of the existing IoT ecosystem and Cloud computing.

The disclosed technology provides scientists and healthcare professions with systems and methods to optimize health by improving health capacity. Rather than viewing health as an elusive concept, the current technology permits it to be viewed as a system with quantifiable, particularized or modularized measurable tasks or targets.

The disclosed technology permits the quantification of health viewed as health capacity, and the detection of its change over time, including changes in magnitude or frequency of circadian or other periodic components. Rather than the current paradigm, viewing the absence of health as the presence of symptomatic disease, the disclosed technology provides for the substantially continuous measurement of health, quantification of its decline, and detection its change prior to the onset of symptomatic disease. Thus, the disclosed technology permits individuals, public health and/or medical professionals to take corrective action with more accurate, reliable information to forestall the onset of disease, decrease morbidity, decrease mortality, and lower the cost of care.

The disclosed technology can also increase the efficiency of currently available precision medicine tools by reducing the false or improper diagnosis rate of discovery, by employing simple, fundamental and translatable metrics of energy usage—an “energy signature,” and thereby reverse the trend of inefficiency according to Eroom's Law.

In some aspects, the disclosed technology relates to methods of detecting recognizable features from the obtainment of physical, chemical, and biological measurements of emergent properties of both biological and non-biological systems; and a system or technology platform that processes, stores, transfers, analyzes, compiles, distributes, and displays emergent property measurement data to quantify, predict, control, maximize, design, and engineer complex adaptive biological and non-biological systems.

In some embodiments, a biological system could be a eukaryotic, prokaryotic, or archaea organism such as a bacteria, gamete or red blood cell, white blood cell, a cell derived from a tissue or organ such as a myocyte, or a simple or complex multicellular organism such as an apple or human, or an engineered cell, and/or a collection thereof to form an interactive ecosystem in conjunction with non-biological essential chemical materials or energy sources.

In some embodiments, a non-biological system could be an engineered non-living system analogous to a biological system but devoid of genetic material—a protocell—or any other engineered or biomimicry-based designed system or semi-synthetic system.

In some embodiments, the disclosed technology further relates to the use of such measures to gauge their state of existence and their ability to persist in biological and non-biological systems. The ability of a biological or non-biological system to exist or persist relates to the system's “health capacity.”

Advantages of the disclosed technology include its ability to quantify, predict, control, maximize, engineer, and design a biological or non-biological system based on measurement of physical, chemical, and biological parameters that relate to the property of resilience (adaptivity) as indicated primarily by its ability to persist or achieve some core function capability.

In some embodiments, the disclosed technology further relates to a technology platform and methods for obtaining physical, chemical, and biological parameters include wearable, implanted, embedded, or otherwise-coupled devices. Once such measurements are implemented, they can be stored, quantified, analyzed, compiled, distributed, and displayed to quantify, predict, control, maximize, engineer, and design a biological system. Measures of health capacity can also be analyzed in conjunction with other measures that are not those of emergent properties to improve upon the ability of the measurement technology and learning platform to quantify, predict, control, maximize, engineer, and design a biological or non-biological system.

In the example of single-celled eukaryotic or prokaryotic or archaea organisms, the disclosed technology would allow for the quantification, prediction, and maximization of function. In the case, for example, of a gamete, the disclosed technology would allow for the quantification of health capacity to maximize fertility for in vivo or in vitro fertilization. In bacteria, yeast or human cells, the disclosed technology could be used for the quantification of health capacity of, in the case of industrial or synthetic biology, the synthesis of molecules. In the case of non-pathogenic enteric bacteria, the disclosed technology could be used to maximize microbiome function.

In the example of multi-celled eukaryotic organisms, the disclosed technology would allow for the quantification, prediction, and maximization of function. For example, in humans the disclosed technology would enable the measurement and maximization of health and the diagnosis and/or pre-symptomatic diagnosis of disease, and the designs of methods for disease treatment or interception, where disease could be infectious, cancer, toxic, iatrogenic, or metabolic, and where health determinants are food/nutrition, sleep, exercise, neuromuscular activation, and changes to lifestyle such as smoking, inactivity, addiction.

In certain embodiments, the advantages of the disclosed technology can further include its applications in non-human multi-celled eukaryotic organisms. For example, in agricultural systems, such as farming of plants and animals. In these biological systems the disclosed technology would enable the maximization of food substance production and/or mitigation of abiotic or biotic stress.

In certain embodiments, the advantages of the disclosed technology can further include its application in simple ecosystems of species and chemical resources. For example, in biological systems of two or more species and one or more resources, the disclosed technology would enable their mutual maximization of functional interdependence. Where one of the species was a human, for example, the technology would enable the maximization of human functional parameters such as sleep, activity, physical or cognitive performance and disease prevention.

In certain embodiments, the advantages of the disclosed technology can further include its applications in complex ecosystems. For example, in biological systems of many species and many resources, the disclosed technology would enable their mutual maximization of functional interdependence. Where the complex system was a farm the disclosed technology would enable maximization of an ecosystem's function such as sustainability.

In certain embodiments, the advantages of the disclosed technology can further include its applications in synthetic biology, for example in eukaryotic and prokaryotic cells. In these biological systems, the disclosed technology would enable design and engineering to maximize function; for example, the synthesis of a protein, lipid, or small molecule or the capability to maintain viability under condition of abiotic or biotic stress.

In certain embodiments, the advantages of the disclosed technology can further include its applications in biometrics. For example, the disclosed technology would enable the identification of a biological system independent of or in conjunction with genetic material.

In certain embodiments, the advantages of the disclosed technology can further include its applications in non-biological systems. For example, the disclosed technology would enable the design and engineering of a non-living system analogous to a biological system but devoid of genetic material—a “protocell.” Such protocell could be used for learning and/or doing work, where work may be understood to mean any output that is not merely heat production.

In certain embodiments, the advantages of the disclosed technology can further include its application in biological systems to quantify biological time—“health span.” For example, the disclosed technology could be used to calculate the theoretical and actual life span(s) of a biological system and be used in conjunction with the aforementioned uses to maximize function or health-span.

In general, thermoregulation consists of two principal components: heat production and heat elimination. Healthy homeostasis requires a balance between the two. There is a dynamic balance in which the two components remain roughly equal through a large range of energy demands and rate of change. By measuring the two components continuously and analyzing their temporal alignment, the system can assess health in a much more detailed manner than if only one or the other were measured.

### EXAMPLES

In certain embodiments, heat flux can be conceptually plotted as a function of ambient temperature. It will be understood by one of ordinary skill in the art that an visual multi-dimensional plot need not be physically generated, but rather the calculations and analyses underlying such a plot can be performed directly. In this manner, there may be insight into how an internal signal and an external signal reflect on the health state of a subject. In certain embodiments, the internal signal and the external signal can be analyzed simultaneously, which may lead to a more in depth reflection of the overall health state of the subject. This can highlight heat as an emergent factor. In certain embodiments systems, methods, and devices may collect and/or generate a data stream that may include a plurality of heat flux data and a plurality of ambient temperature data. In certain embodiments, the heat flux data may include a plurality of heat elimination measurements expressed as a function of environmental temperature. In certain embodiments, systems, methods or devices may include a processor that may receive the data stream. In certain embodiments, systems, methods, and devices may detect features or shapelets that may be related to and/or correlate with a health state or status of an individual. In certain embodiments, the feature detected may represent a state of homeostasis. A state of homeostasis may be represented by a densely populated region within the data stream. This can be an example of a motif detected by the system. In some embodiments, the state of homeostasis can be detected by the system before or after a novelet detected by the system. In some embodiments, a more densely populated region within the data stream may represent that the health state of the subject may be maintained at that point in time. In certain embodiments, a less dense, more diffuse an area of the data stream is the system may determine the less stable or more unstable a thermal system of a subject may be. This may indicate a novelet may be forming, of which the system may detect. In certain embodiments, the system may detect an overall feature or snippet of the plurality of data collected by detecting the overall structure of the data. In certain embodiments, the system may detect sub features or shapelets within the overall pattern of the plurality of data.

In certain embodiments, an overall pattern that the system may detect may be a boomerang shape, L-shape, or the like when conceptually plotted as heat flux vs. environmental temperature. In certain embodiments, the system may identify a peak or turning point of the overall shape detected. The system may detect a sub pattern within the overall shape detected. For example, the system may detect the degree or pronunciation of the turning or transition point detected within the overall shape detected. In certain embodiments, the detected degree or pronunciation of the turning point may be indicative of age, health system efficiency, homeostasis, ability to obtain homeostasis in varying environments, etc. For example, the system may detect a more defined turning point, which may be reflective of a younger subject or one with greater cardiorespiratory fitness. In certain embodiments, the system may detect sub features that may be located at the ends of the overall shape detected and/or within the overall shape detected. The sub features may include variability within the data which may correlate to a recognizable feature. For example, the system may detect a sub feature that may be indicative of a subject's ability to tolerate cold temperatures, a subject's cardiac function, a subject's prognosis, etc.

In certain embodiments, the system may detect a feature associated with a health state of a subject. In certain embodiments, the health state may be associated with a stage of a health state or response to an infection, disease, etc. This can be based on the amount of heat the subject is conserving or eliminating at a given environmental or skin temperature. In certain embodiments, the system may detect a recognizable feature that is based on the amount of heat eliminated from the subject at a given temperature. In some embodiments, the system may detect a recognizable feature for each disease, infection, health related illness, etc. Within each detectable recognizable feature, the system may determine the subject's response to the disease, infection, or health related illness. Additionally, the system may predict, based on the detectable recognizable feature, the subject's future response to the disease, infection, or health related illness. For example, the system may determine whether the subject is healing from the illness, fighting the illness, or responding to the illness. In certain embodiments, the system may detect when the subject transitions between each stage of responding to the illness. In some embodiments, the detection of the transition states can allow a subject to gain an understanding as to the timeline of the disease, illness, or condition and then prepare for accordingly. In certain embodiments, this understanding may be provided to the subject in a minimal invasive manner without compromising the strength of the data stream signal.

In certain embodiments, a recognizable feature may be the maintenance of homeostasis. This may be where the thermoregulatory system of the subject spends the most time. While the system may detect movement, the sub feature detected by the system may be indicative of the maintenance of homeostasis. This can include the delta or change in position at any time, independent of direction, indicating that there is a change in heat flux.

In certain embodiments, the more data collected, the more indicative the detected recognizable feature may be representative of a condition, illness, disease, etc. This may allow the system to forecast future features it may detect based on the previously recognized features within the data stream. This may also allow the system to classify the data stream to a specific class of behavior based on the detected recognized features.

In certain embodiments, the recognizable features may be generalizable to a greater population. For example, the overall pattern of the data may be generalizable to a population of subjects with the same or similar disease or illness. Additionally, for example, the sub pattern of the data may be generalizable to a greater population for how a subject's thermoregulatory system may fluctuate over time. With generalizations, the system may be able to determine anomalies or outliers to provide the subject with additional information. The anomalies may be found amongst the subject's own features detected by the system or the features of a greater population.

In certain embodiments, a user may use the detected recognizable features by the system in various manners. For example, a user may use the detected recognizable feature for early detection of a disease. This may include determining a nominal heat flux of a patient, measuring a current heat flux of the patient, and comparing the current heat flux of the patient to the nominal heat flux of the patient to detect disease. In another example, a user may use the detected recognizable features for recommending a thermal regulation guideline. This may include determining a current phase of a menstrual cycle of the user, measuring a current heat flux of the user, and comparing the current phase of the menstrual cycle with the current heat flux to determine a thermal regulation guideline. In another example, a user may use the detected recognizable features for determining readiness of a warfighter. This may include determining a current state of a potential warfighter, measuring a current heat flux of the potential warfighter, and comparing the current heat flux of the potential warfighter with the current state of the potential warfighter to determine the readiness of a warfighter.

In another example, a user may use the detected recognizable features for regulating sleep. This may include determining a current circadian rhythm of a user, measuring a current heat flux of the user, and comparing the current heat flux of the user with the current circadian rhythm of the user to regulate sleep of the user. In another example, a user may use the detected recognizable features for determining readiness of a user to conceive. This may include determining a current phase of a menstrual cycle of the user, measuring a current heat flux of the user, and comparing the current phase of the menstrual cycle of the user with the current heat flux to determine the user's readiness to conceive. In another example, a user may use the detected recognizable features for determining a stage of pregnancy of a user. This may include determining a current state of the user, measuring a current heat flux of the user, and comparing the current state of the user with the current heat flux of the user to determine the state of pregnancy of the user.

In another example, a user may use the detected recognizable features for determining gut health of a user. This may include determining a current phase of digestion of at least one food of the user, measuring a current heat flux of the user, and comparing the current phase of digestion with the current heat flux to determine gut health of the user. In another example, a user may use the detected recognizable features for determining athletic capabilities of a user. This may include determining a current state of health capacity of a user, measuring a current heat flux of the user, and comparing the current state of health capacity of the user with the current heat flux of the user to determine athletic capabilities of the user. In another example, a user may use the detected recognizable features for determining metabolic rate of a user. This may include determining a current amount of muscle mass of a user, measuring a current heat flux of the user, and comparing the current amount of muscle mass of a user with the current heat flux of the user to determine the determining a metabolic rate of a user.

In another example, a user may use the detected recognizable features for determining a syncope event of a user. This may include determining current physical state of the user, measuring a current heat flux of the user, and comparing the current physical state of the user and the current heat flux of the user to determine an upcoming syncope event. In another example, a user may use the detected recognizable features for determining health capacity of a user. This may include determining a current quasiperiodic rhythm of the user, measuring a current heat flux of the user, and comparing the current quasiperiodic rhythm of the user with the current heat flux of the user to determine health capacity of the user. In another example, a user may use the detected recognizable features for determining heart function efficiency of a user. This may include determining a current heart capacity of the user, measuring a current heat flux of the user, and comparing the current heart capacity with the current heart flux to determine heart function efficiency of the user.

In another example, a user may use the detected recognizable features for determining immune system efficiency. This may include determining a current state of immune system of a user, measuring a current heat flux of the user, and comparing the current state of the immune system of the user with the current heat flux of the user to determine the efficiency of the immune system of a user. In another example, a user may use the detected recognizable features for determining a user's weight loss capabilities. This may include determining a current weight of the user, measuring a current heat flux of the user, and comparing the current weight of the user with the current heat flux of the user to determine the user's weight loss capabilities. In another example, a user may use the detected recognizable features for determining medication effectiveness to treat a disease. This may include determining a current stage of a disease, measuring a current heat flux of the user, and comparing the current stage of the disease with the current heat flux of the user to determine the effectiveness of a medication to treat the disease.

In another example, a user may use the detected recognizable features for determining a risk of loss of pregnancy. This may include determining a current stage of pregnancy, measuring a current heat flux of the user, and comparing the current stage of pregnancy with the current heat flux of the user to determine the risk of loss of pregnancy. In another example, a user may use the detected recognizable features for determining a level of sobriety of a user. This may include determining a current amount of alcohol consume by the user, measuring a current heat flux of the user, and comparing the current amount of alcohol consumed by the user with the current heat flux of the user to determine the level of sobriety of the user. In another example, a user may use the detected recognizable features for determining a recovery regimen for a user. This may include determining a current stage of an injury, measuring a current heat flux of the user, and comparing the current stage of an injury with the current heat flux of the user to determine the recovery regiment for the user. In another example, a user may use the detected recognizable features for recommending a thermal regulation guideline to a user. This may include determining a current phase of menopause of the user, measuring a current heat flux of the user, and comparing the current phase of menopause with the current heat flux to determine a thermal regulation guideline.

In certain embodiments, subjects are classified in one of at least two groups: for example, as having a positively functioning thermoregulatory system or as having a negatively functioning thermoregulatory system. In some embodiments, subjects may lie in between the two groups as well. In other words, the functional level (functionality) of a subject's thermoregulatory system or the efficiency at which a subject's thermoregulatory system functions at may exist on a spectrum. In some embodiments, the functionality of a subject's thermoregulatory system may correlate with the resilience of the subject's thermoregulatory system. In some embodiments, the functionality of a subject's thermoregulatory system may be based on a health status of the subject. The resilience of the subject's thermoregulatory system, for example, may be related to the ability of the subject to control its thermoregulatory system, the ability of the subject's thermoregulatory system to return to homeostasis if it veers from its homeostatic state, or the ability of subject's thermoregulatory system to return to homeostasis in an ordered manner or fashion. In other words, there may be a plurality of thermoregulation measurements that make up a transitory phase of the thermoregulation system. The thermoregulation system may be in a transitory phase when the thermoregulation system veers from homeostasis. Homeostasis may be considered the normal state of the thermoregulation system of a subject. The resilience of a subject's thermoregulatory system may be based on the ability of the thermoregulatory system to control itself when it veers from the normal state to a transitory phase over a period of time. The resilience of the thermoregulatory system may be related to the duration, efficient manner, or lack thereof, of which the subject's thermoregulatory system returns to its homeostatic or normal state. In some embodiments, the way in which the subject's thermoregulatory state returns to its homeostatic or normal state can be associated with a phenotype. The phenotype associated with the way in which the subject's thermoregulatory state returns to its homeostatic or normal state may allow various biological measurements to be used to assess the thermoregulatory system of the subject. For example, heart rate, heat flux, skin temperature, or the like may be used to assess the thermoregulatory system of the subject.

In some embodiments, a non-resilient thermoregulatory system does not return to its homeostatic or normal state if it remains in a transitory state or veers from its homeostatic or normal state. In some embodiments, a non-resilient thermoregulatory system may return to its homeostatic state over an extended period of time. In other words, a non-resilience thermoregulation system may remain in a transitory state for an extended period of time. This may mean that the thermoregulation system may not be able to control itself. In some embodiments, a non-resilient thermoregulatory system may return to its homeostatic or normal state in an unordered manner or fashion.

In some embodiments, the resilience of the thermoregulatory system of a subject is assessed. The assessment of the resilience of the thermoregulatory system may lead to a recommendation provided to the subject. In some embodiments, the recommendation may include a recommendation for the subject to engage in an intervention based on the assessed resilience of thermoregulation. In some embodiments, a thermoregulation resilience score may be generated for the subject over a pre-determined time period. In some embodiments, the thermoregulation resilience score may be based on a data stream that includes a plurality of thermoregulation measurements of the subject. The plurality of thermoregulation measurements may be collected from any of the devices described above. In some embodiments, the plurality of thermoregulation measurements may include any biological measurement that can be related to the thermoregulatory system of a subject.

The assessment may include determining whether a need for intervention as to the subject is above a predetermined threshold of urgency. A recommendation to engage in the intervention as to the subject may be generated as well. In this manner the recommendation may be tailored to or based on the thermoregulation resilience score, the need of intervention, or a combination thereof.

In some embodiments, the threshold of urgency may be related to the urgency of which an intervention as to the subject may be needed. For example, the higher the thermoregulation resilience score, the less urgent the intervention as to the subject may be. In some embodiments, a predetermined threshold of urgency may determine the urgency of the intervention as to the subject needed. For example, the thermoregulation resilience score of a subject may be greater than a predetermined threshold of urgency, which may lead to an assessment that an intervention as to the subject is not as urgent as an intervention for a subject whose thermoregulation resilience score is less than the predetermined threshold of urgency. In some embodiments, the predetermined threshold of urgency may be subject specific. For example, the predetermined threshold of urgency may be based on or vary based on the health status of the subject.

In some embodiments, it may be useful to have a metric of health that is refreshed, reanalyzed, recalculated, observed, and/or reconfigured every month, three weeks, 14 days, 10 days, 7 days, 5 days, 3 days, 2 days, 1 day, daily, less than one day, or any measure of time in in between. This may be because the health of the subject can change over time. The frequency of which the metric of health is calculated or observed may allow the subject to quantify a transitory phenomenon that can exist in the health of a subject. For example, the transitory phenomenon may include a transition phase, or the transition or transitory state described above. Similarly, the transitory phenomenon may include the resilience of the subject's thermoregulatory system and its ability to respond to a factor that causes the thermoregulatory system to veer from its homeostatic or normal state. This may relate to the homeostatic robustness of the subject. In other words, the homeostatic robustness of the subject may be based on the resilience of the thermoregulation system of the subject. In some embodiments, the homeostatic robustness may contribute to the pattern recognized within the plurality of data or characterization of the plurality of data. In some embodiments, the more resilient the thermoregulation system of subject is, the greater homeostatic robustness of the subject may be. In some embodiments, the transitory phase may exist for a period of time. The period of time can be any length of time. For example, the transitory phase may exist for an extended period of time, a non-extended period of time, or for a pre-determined period of time. In some embodiments, if the transitory period exists for an extended period of time, then the transitory phase may indicate a non-resilient thermoregulation system of the subject. This may be because the thermoregulatory system of the subject has less control over itself when it veers from its normal state. In some embodiments, if the transitory period exists for a non-extended period of time, then the transitory phase may indicate a resilience thermoregulation system of the subject.

In some embodiments, the assessment and generation of a recommendation may include alerting a medical practitioner that a recommendation has been generated. This may alert the medical practitioner to intervene as to the subject as necessary or as recommended.

In some embodiments, the thermoregulation resilience score may be viewed in combination with other biological measurements. This may allow for a greater probability of identifying a subject that may have a health status that may be in need of or may be at risk for treatment or intervention. For example, comparing the thermoregulation resilience score against the thermoregulation resilience score in combination with other biological measurements can better identify a subject that may have a health status that is in need of or at risk for treatment or intervention. This may be due to the positive correlation between the resilience of a subject's thermoregulation system and the health status of the subject. The correlation may further be supported by the other biological measurements of the subject. In some embodiments, the other biological measurements may be collected from any a device that can sense and/or collect biological measurements.

In some embodiments, a plurality of thermoregulation resilience scores of a plurality of subjects may be plotted against a combination of a plurality of thermoregulation resilience scores and a plurality of other biological measurements. The combination may be considered a risk rank. The lower the risk rank, the greater risk the subject may have. In some embodiments, by allowing the risk rank to include both thermoregulation resilience scores and other biological measurements, there may be a greater probability of identifying a subject that may have a health status that is considered at risk. For example, this risk assessment can increase the probability of identifying a subject at risk by approximately 2-2.5 fold.

In some embodiments, the risk assessment may be tailored to remove at least one outlier that maybe included in the plot of the combination of a plurality of thermoregulation resilience scores and a plurality of other biological measurements. The at least one outlier that may be removed may include an outlier that is on a high end of the risk rank scale and a low end of the risk rank scale. By removing the outliers, the probability of identifying a subject with a health status that is considered at risk may increase by approximately 4-5 fold.

In some embodiments, the risk assessment discussed above may be used to identify serious medical events in fragile elderly subjects. For example, the fragile elderly subject may be constantly assessed for risk by constantly collecting a plurality of biological measurements and calculating the thermoregulation resilience score of the fragile elderly subject. The risk assessment described above can be used to predict the odds of a medical event occurring. Additionally, the risk assessment described above can be used to triage outpatient care resources, which can multiply the effective capacity of skilled medical care practitioners. This may decrease the morbidity associated with aging. This may also decrease the cost of related hospitalization by identify a health risk of an individual prior to a medical event occurring.

In some embodiments, the risk assessment discussed above may be used to determine medical insurance for a younger population that may be considered healthy. For example, in general, the younger population may need minimal medical care except in cases of medical events, traumatic injury, and/or unexpected diseases. By assessing the risk of the young population as described above, subjects of the younger population that are at risk or have physiological risk factors may be identified. By identifying these subjects, the subjects may be segmented into various subgroups of various risks. The subgroups may then be able to be incentivized to screen themselves to identify the subjects within the subgroup that are at a higher risk than others.

In some embodiments, the risk assessment discussed above may be used to identify workers or laborers that are at a higher risk for heat stroke than others. For example, workers or laborers that may be working in hot conditions may be at a higher risk for heat stroke than other individuals. In general, businesses may impose a set limit that workers or laborers may not work when the outdoor temperature exceeds a certain temperature threshold. However, some individuals may have a risk for heat stroke at a lower temperature threshold. This may be an all or nothing strategy. By identifying subjects that have a higher risk for heat stroke than others using the above described risk assessment, targeted work management may be implemented. This may lead to less downtime for the business, workers and/or laborers. This may also lead to less cost from injury of the workers or laborers.

FIG. 1A and FIG. 1B illustrate an example embodiment of the system detecting recognizable features from a subject's response to an LPS injection. Because each figure illustrates the same or similar recognizable feature identified by the system in two different subjects who are otherwise healthy subjects, FIG. 1A will be described, however the description will also apply to FIG. 1B. The thermoregulation response to an LPS injection can be seen in FIG. 1A. As shown, the system can analyze the data generation by grouping the data into bins that encompass a period of about every 5 minutes. Upon an injection of LPS, the system may detect a decrease in heat flux or heat elimination. This may be because the thermoregulation system of the subject is trying to retain heat due to the LPS injection; by retaining heat, the subject may initiate a fever to try and fight the infection. As shown in FIG. 1A, the system may detect a recognizable sub feature associated with the LPS injection and/or transition to retention of heat. The recognizable sub feature detected may be a downward trend. The system may detect the thermoregulatory system continuing to retain heat by identifying a recognizable feature created by the retention of heat by the subject. As shown in FIG. 1A, the system may detect another recognizable sub feature upon the thermoregulatory system of the subject releasing the heat it had previously retained. The recognizable sub feature detected may be an upward trend. The system may detect the recognizable feature as eliminating a greater amount of heat than normal. This may be because the thermoregulatory system of the subject had previously retained more heat than normal, so more heat may need to be eliminated. As shown in FIG. 1A, the overall recognizable feature may be J-shape when plotted as heat flux as a function of ambient temperature. The overall recognizable feature detected by the system may be created by at least one transition stage before and after each recognizable sub feature. As further shown in FIG. 1A, the overall recognizable feature detected may be presented in a counterclockwise direction.

FIG. 2A, FIG. 2B, and FIG. 2C illustrate an example embodiment of recognizable features detected by the system from a circadian rhythm of a subject who is otherwise health for a night's sleep. The detected recognizable features may be illustrated by a plot containing heat flux as a function of ambient temperature. Because each figure illustrates the same or similar recognizable features identified by the system in three different subjects who are otherwise healthy subjects, FIG. 2A will be described, however the description will also apply to FIG. 2B and FIG. 2C. As shown, similar to FIG. 1A, the system can group data points into bins spanning about every 5 minutes. As shown in FIG. 2A the system may detect and increase heat retention as the subject is falling asleep. The system may detect a recognizable sub feature of a downward trend that indicates the subject is falling asleep. As the subject stays asleep, the system may detect a recognizable sub feature that indicates the subject is sleeping. The recognizable feature may be a cluster of data points in the bottom right quadrant of the plot. As the subject wakes, the system may detect a recognizable sub feature that indicates the subject is waking. The system may detect the recognizable sub feature as an upward trend, indicating the subject is releasing the previously retained heat. As the subject remains awake, the system may detect a recognizable sub feature that indicates the subject has awoken. The recognizable feature may be a cluster of data points in the upper right quadrant. While the system can detect a recognizable sub feature for each stage of the sleep cycle, the system can also detect an overall recognizable feature for the sleep cycle. As shown, for example, the recognizable feature for the sleep cycle of a healthy person may be an ellipse. Furthermore, the ellipse may have a counter-clockwise rotation when plotted as in FIG. 2A.

FIG. 3A, FIG. 3B, and FIG. 3C illustrate an example embodiment of recognizable features detected by the system from a circadian rhythm of a subject who is otherwise unhealthy for a night's sleep. The detected recognizable features may be illustrated by a plot containing heat flux as a function of ambient temperature. Because each figure illustrates the same or similar recognizable feature identified by the system in three different subjects who are otherwise considered unhealthy subjects, FIG. 3A will be described, however the description will also apply to FIG. 3B and FIG. 3C. As in FIGS. 1A and 2A, as shown, the system may group data points into bins spanning about every 5 minutes. As shown in FIG. 3A the system may detect an increase in heat retention as the subject is falling asleep. The system may detect a recognizable sub feature of a downward trend that indicates the subject is falling asleep. As the subject stays asleep, the system may detect a recognizable sub feature that indicates the subject is sleeping. However, the unhealthy subject may not be able to regulate heat in the same way as a healthy subject, as described in FIG. 2A, the system may detect a recognizable sub feature that indicates the subject is more awake than asleep. This can result in a recognizable sub feature that is a wide range of a cluster of data points in the top left quadrant of the plot. As the subject wakes, the system may detect a recognizable sub feature that indicates the subject is waking. The system may detect the recognizable sub feature as an upward trend, indicating the subject is releasing the previously retained heat. As the subject remains awake, the system may detect a recognizable sub feature that indicates the subject has awoken. The recognizable sub feature may be a cluster of data points in the upper left quadrant. While the system can detect a recognizable sub feature for each stage of the sleep cycle, the system can also detect an overall recognizable feature for the sleep cycle. As shown, the recognizable feature for the sleep cycle of an unhealthy person may be an inverted triangle. This may be because the unhealthy subject does not retain heat well, and therefore eliminates more heat than it can retain.

FIGS. 4A, 4B, 4C, and 4D illustrate an example embodiment of recognizable features detected by the system from exercise in a subject that is considered otherwise healthy. As shown in FIG. 4A, the system can detect a recognizable feature that is indicative of the subject transitioning from sleep to awake. The recognizable feature may be recognized in a counterclockwise direction starting in the bottom right quadrant of the plot and trending upward to indicate the subject may be eliminating heat. As shown in FIG. 4B, the system can detect a recognizable feature that is indicative of the subject hiking up a steep slope. The recognizable feature may be a cluster of data points in the upper left quadrant. This may be because the thermoregulatory system of the subject may be eliminating heat to maintain their thermoregulatory homeostasis. As shown in FIG. 4C, the system can detect a recognizable feature that is indicative of the subject resting after exertion of energy. The recognizable feature may be a widespread cluster of data points in the bottom right quadrant of the plot. This may be indicative of the subject retaining heat after elimination of heating during the exertion of energy. As shown in FIG. 4D, the system can detect a recognizable feature that is indicative of the subject exerting energy during exercise. The recognizable feature may be a cluster of data points in the upper right quadrant of the plot. This may be indicative of the subject eliminating heat to regulate its thermoregulatory system during exertion of energy.

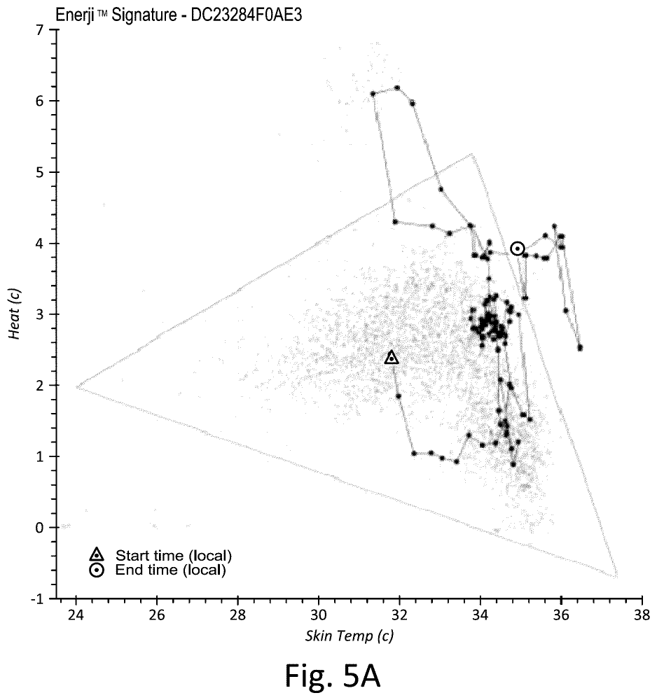

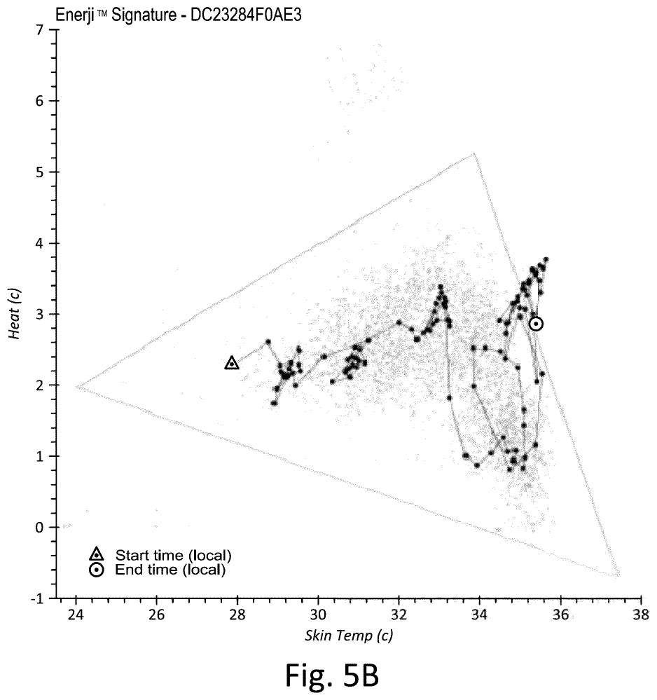

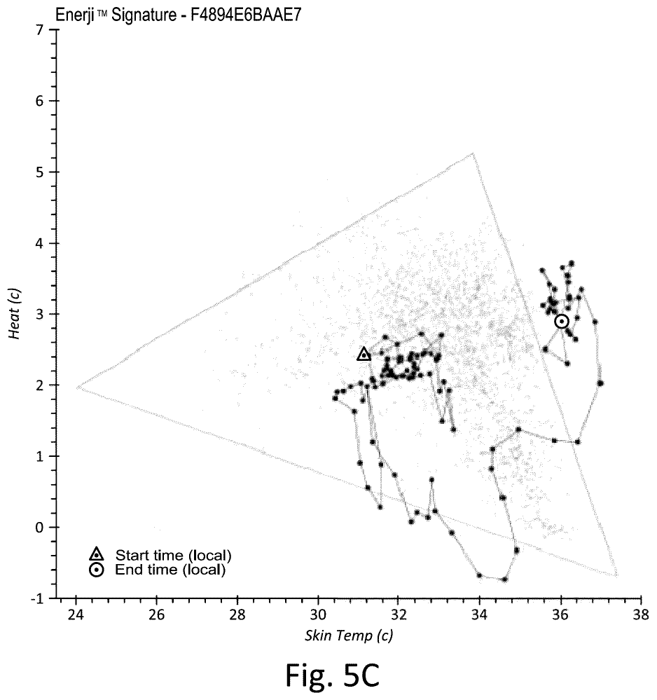

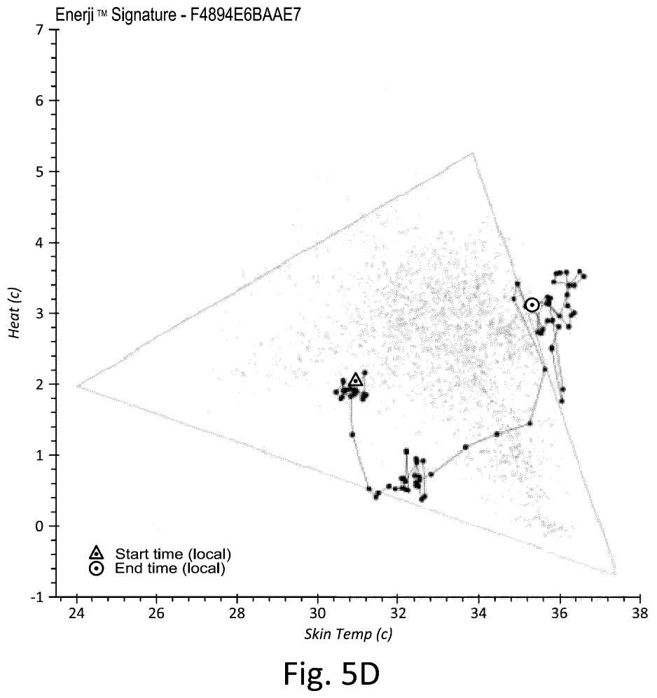

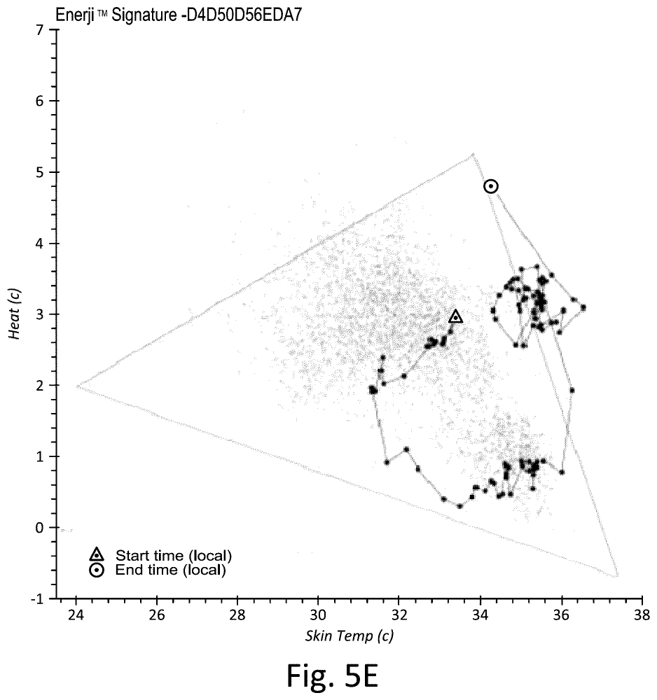

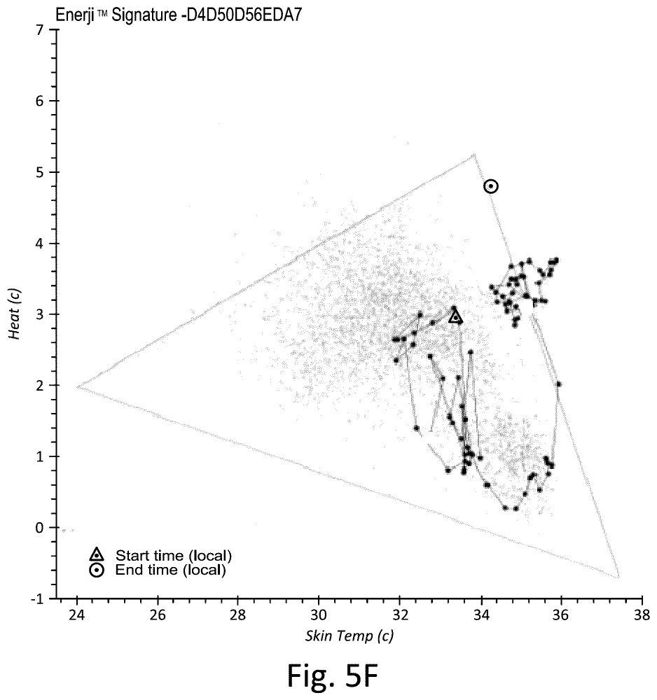

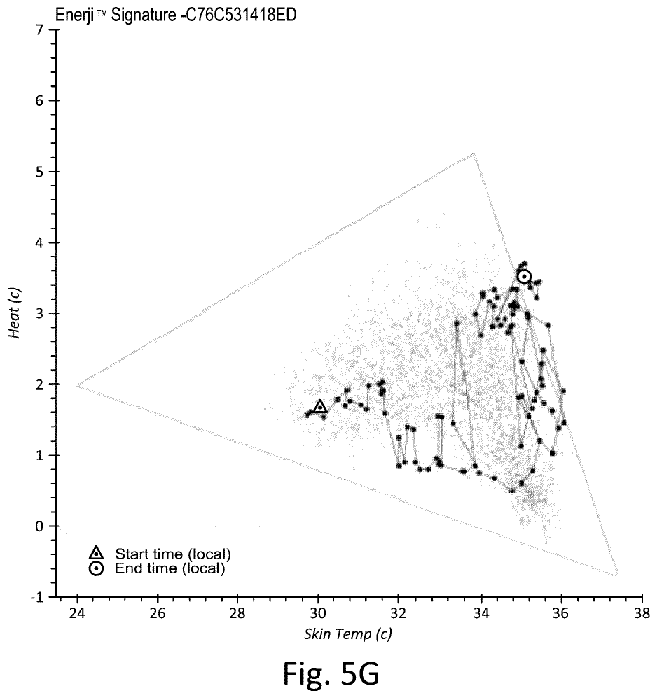

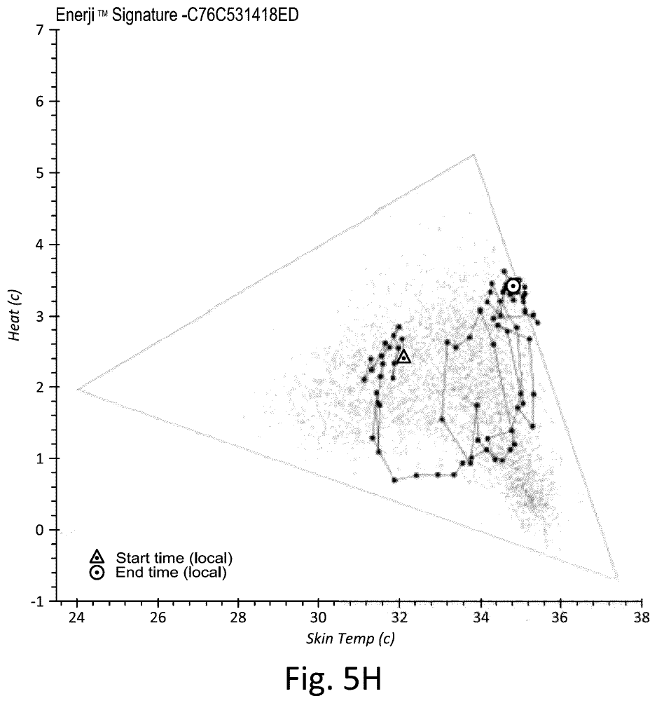

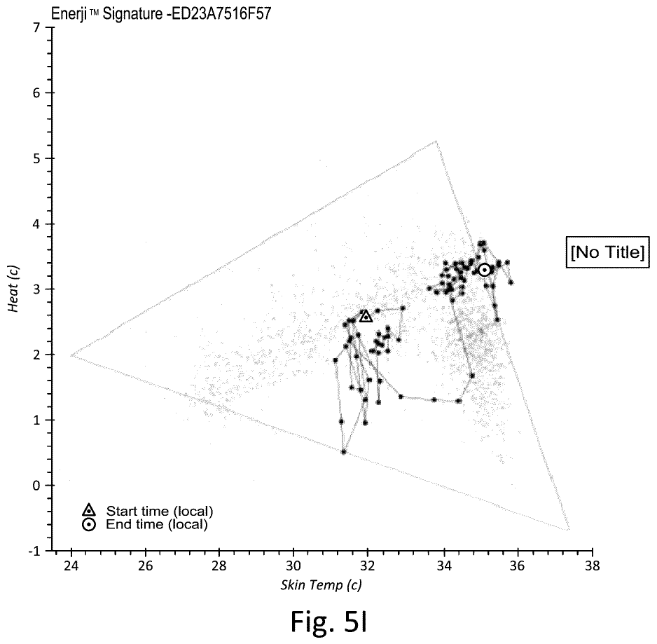

FIGS. 5A, 5B, 5C, 5D, 5E, 5F, 5G, 5H, 5I illustrate an example embodiment of recognizable features detected by the system from inflammation response in a subject.

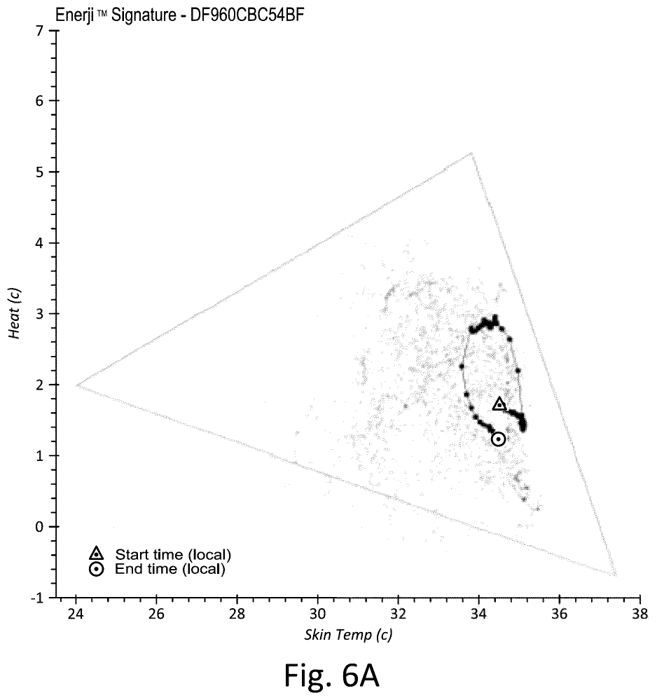

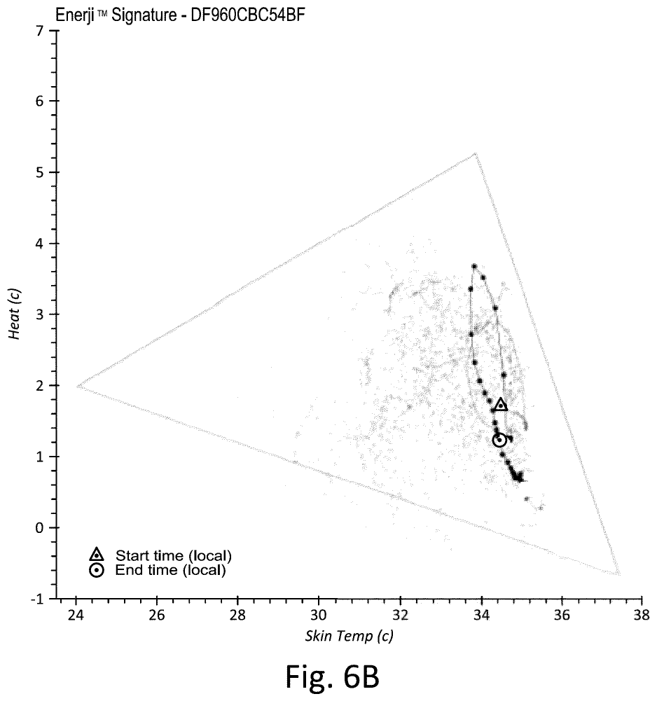

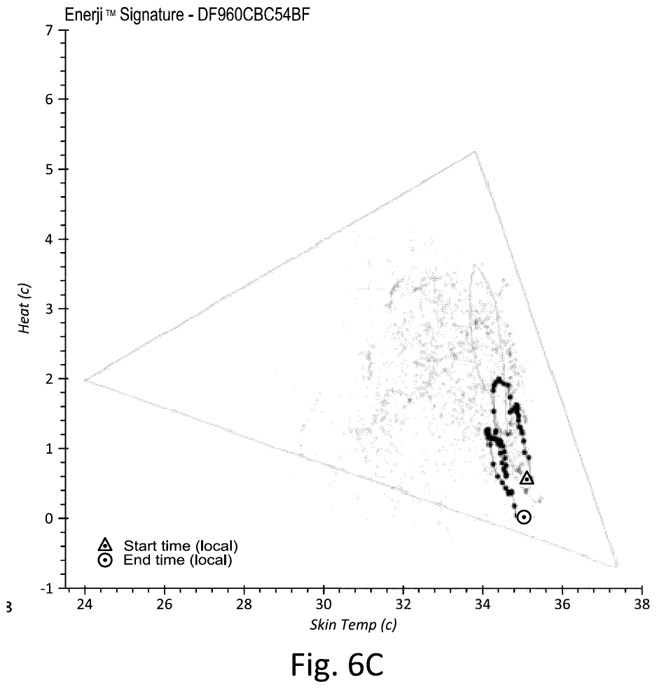

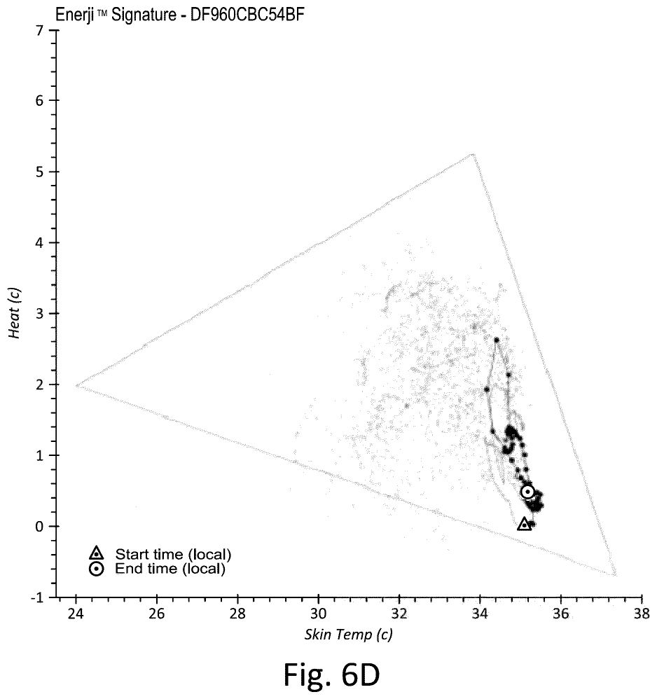

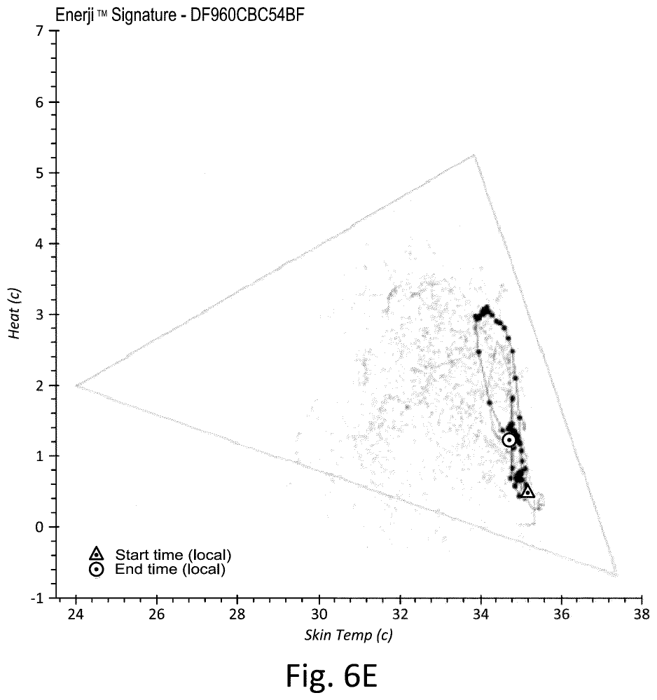

FIGS. 6A, 6B, 6C, 6D, 6E illustrate an example embodiment of recognizable features detected by the system from sleep sequences in a subject that is considered otherwise healthy.

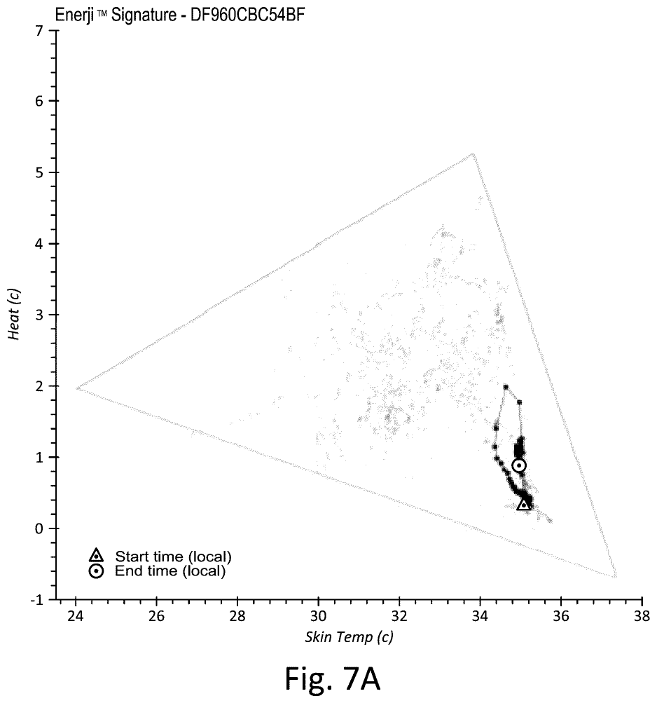

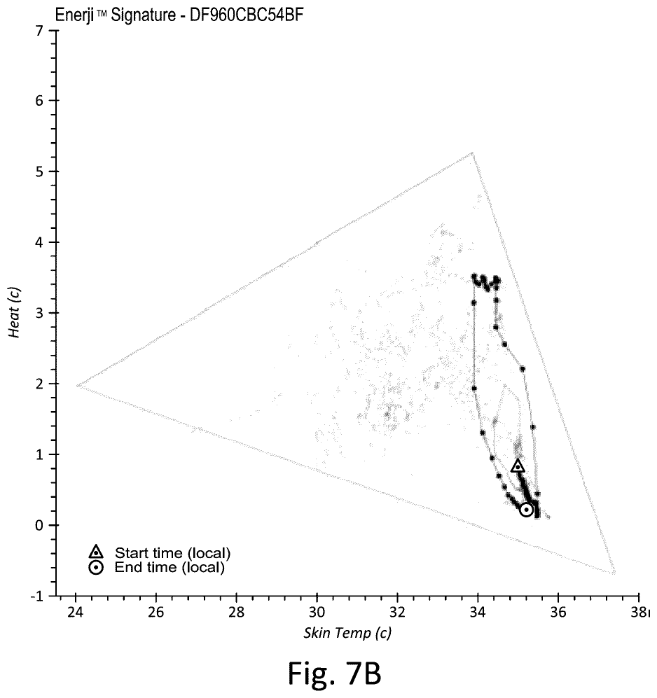

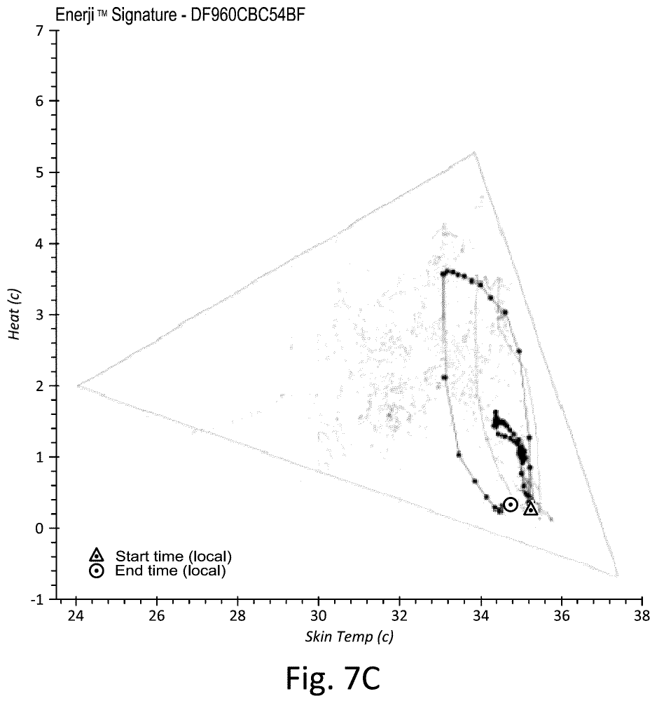

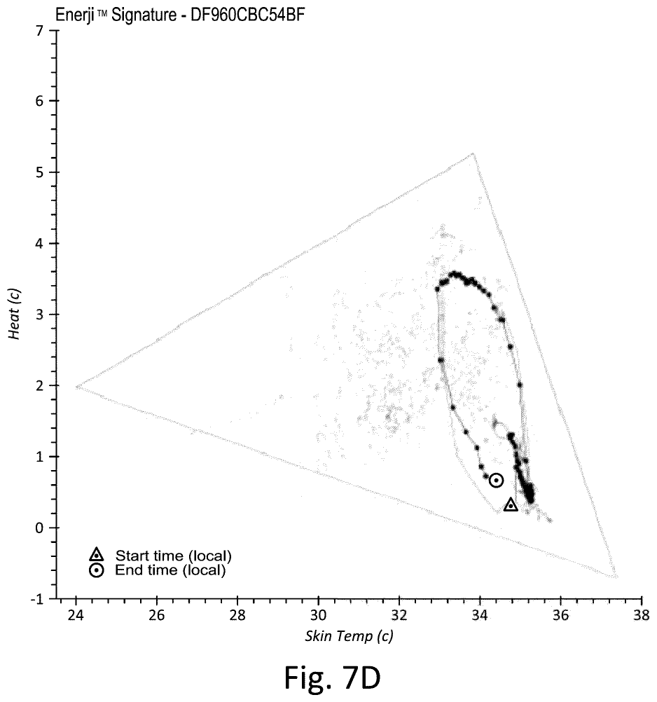

FIGS. 7A, 7B, 7C, 7D illustrate an example embodiment of recognizable features detected by the system from sleep sequences in a subject that is considered otherwise healthy.

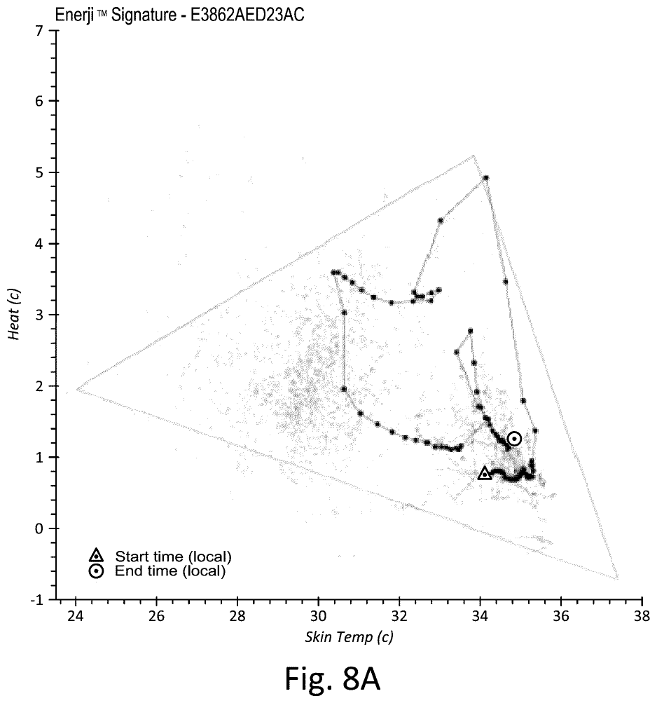

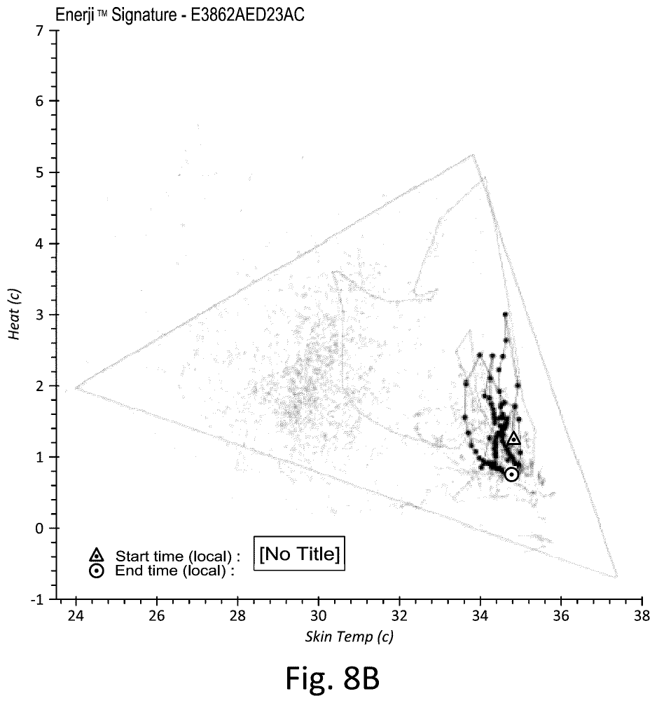

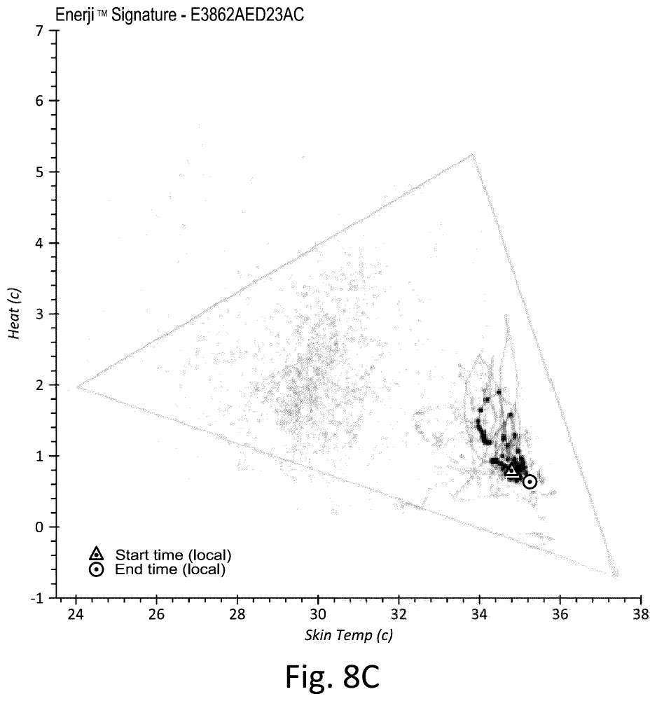

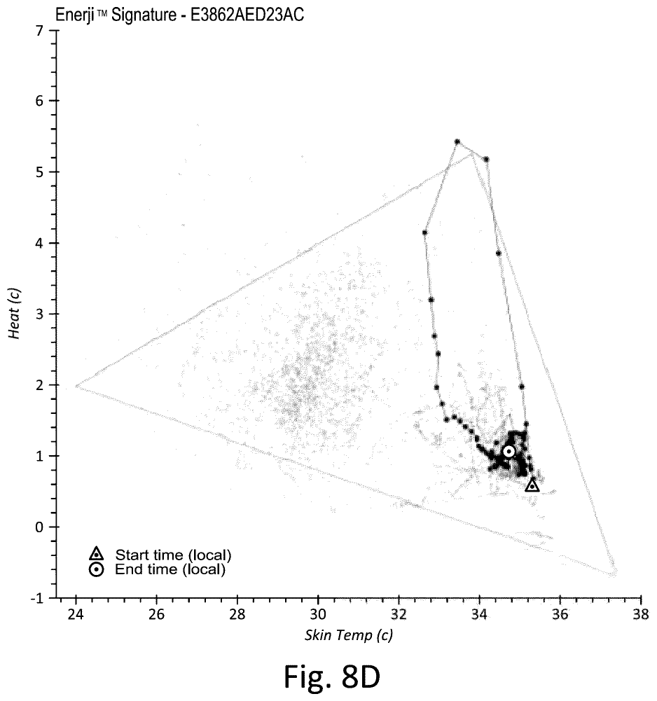

FIGS. 8A, 8B, 8C, 8D illustrate an example embodiment of recognizable features detected by the system from sleep sequences in a subject that is considered otherwise healthy.

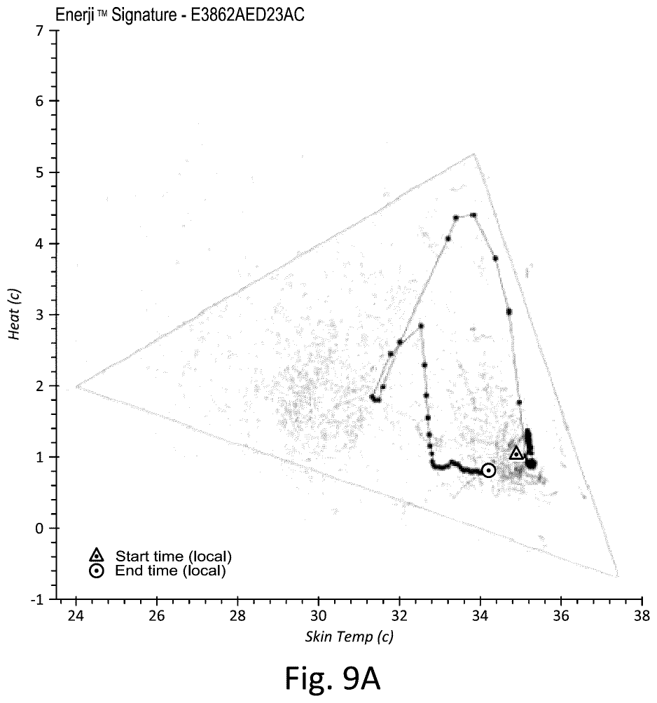

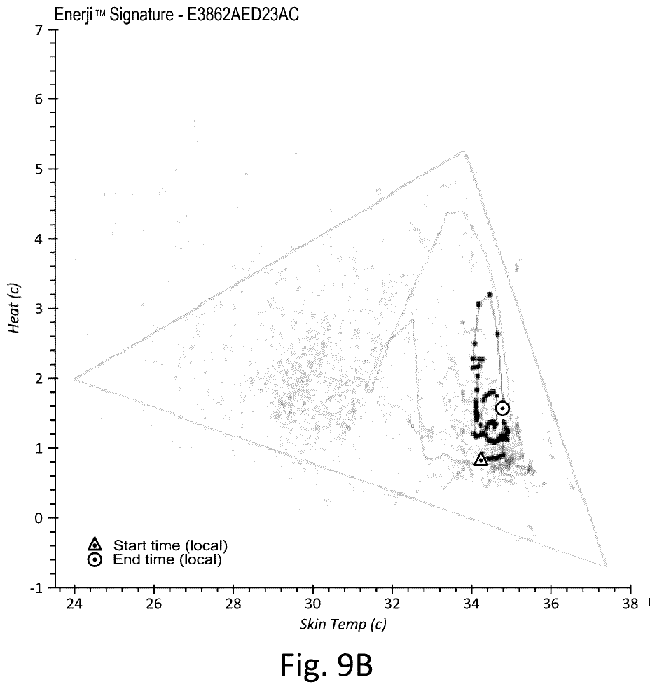

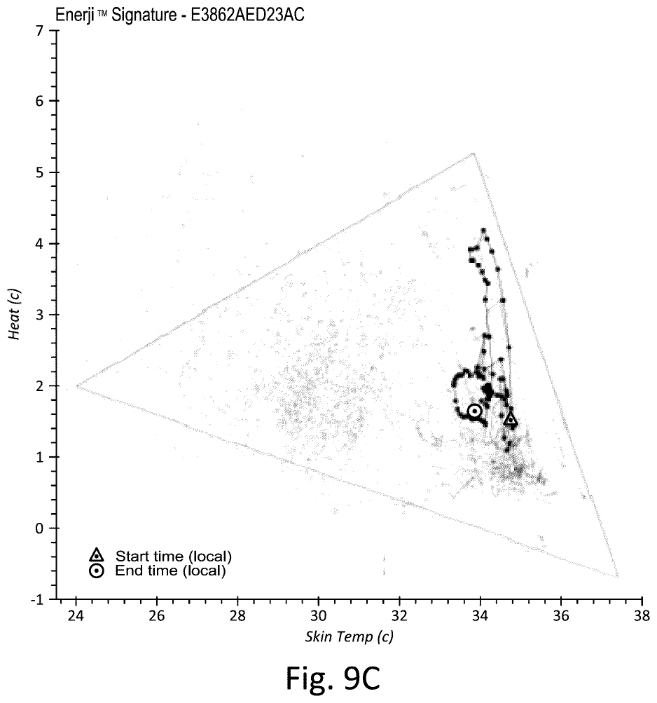

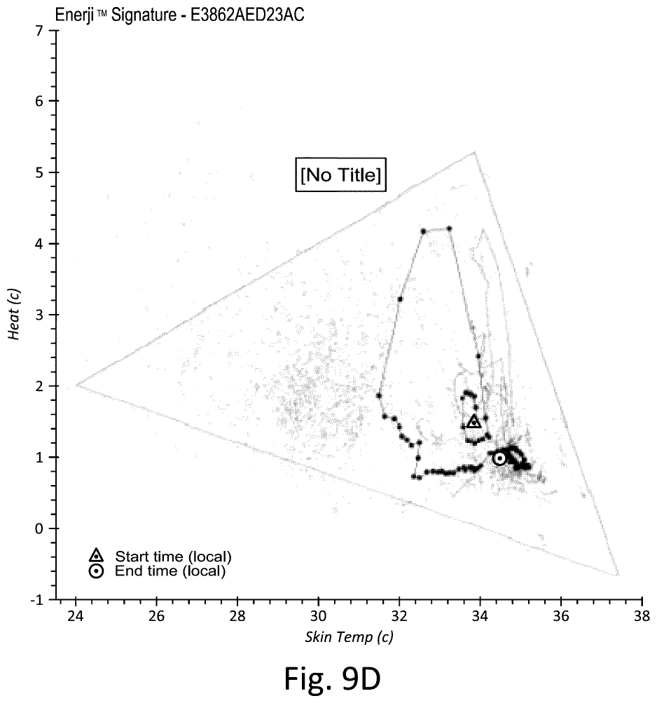

FIGS. 9A, 9B, 9C, 9D illustrate an example embodiment of recognizable features detected by the system from sleep sequences in a subject that is considered otherwise healthy.

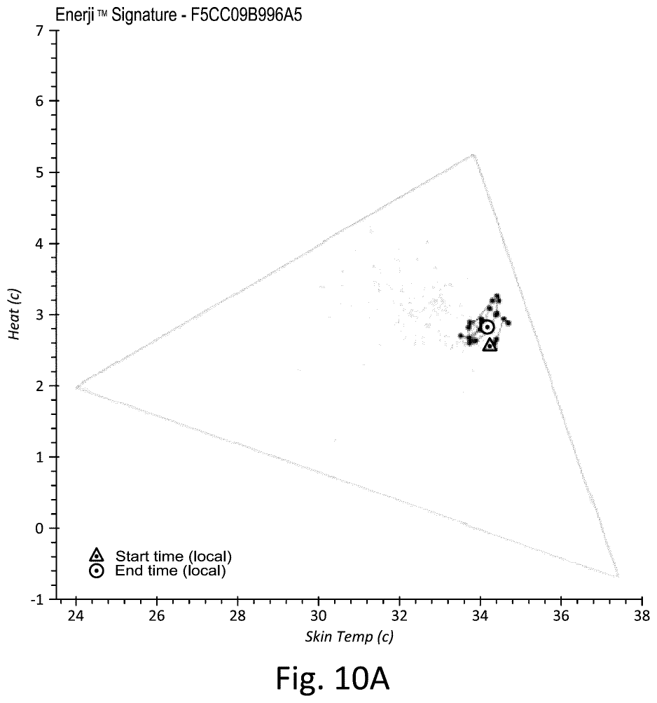

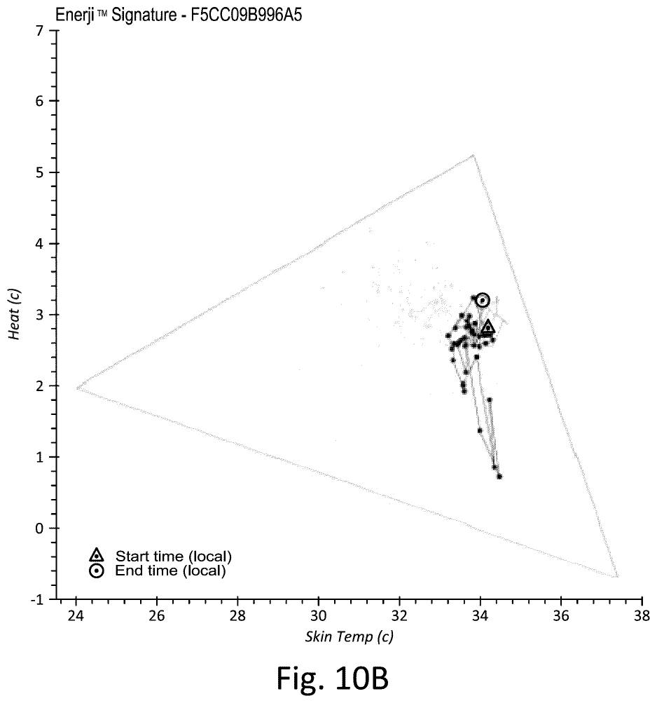

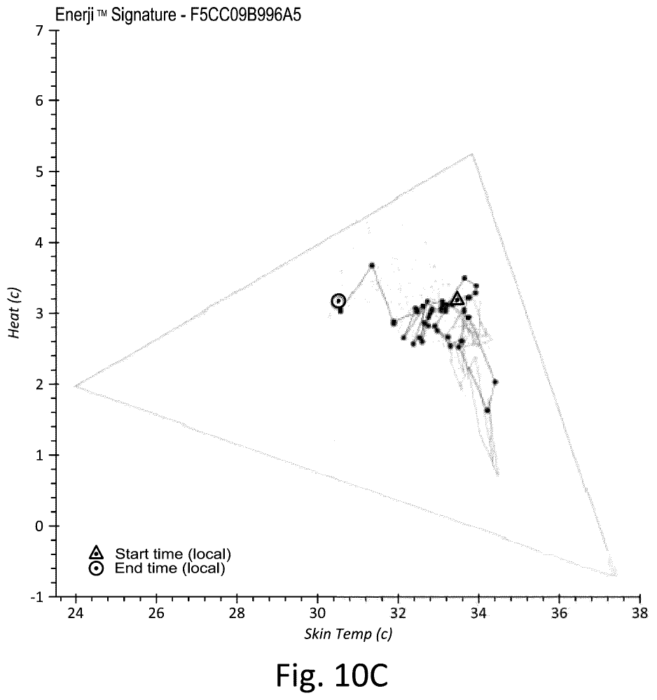

FIGS. 10A, 10B, 10C illustrate an example embodiment of recognizable features detected by the system from sleep sequences in a subject that is considered otherwise unhealthy.

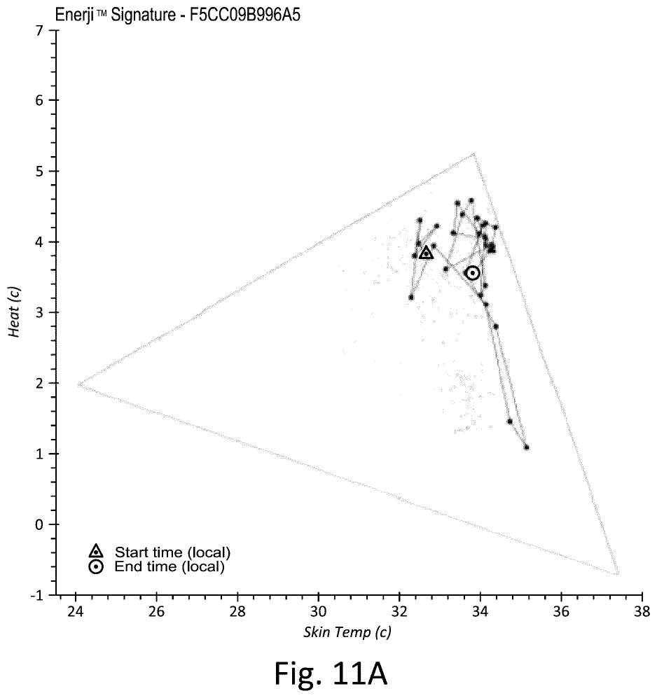

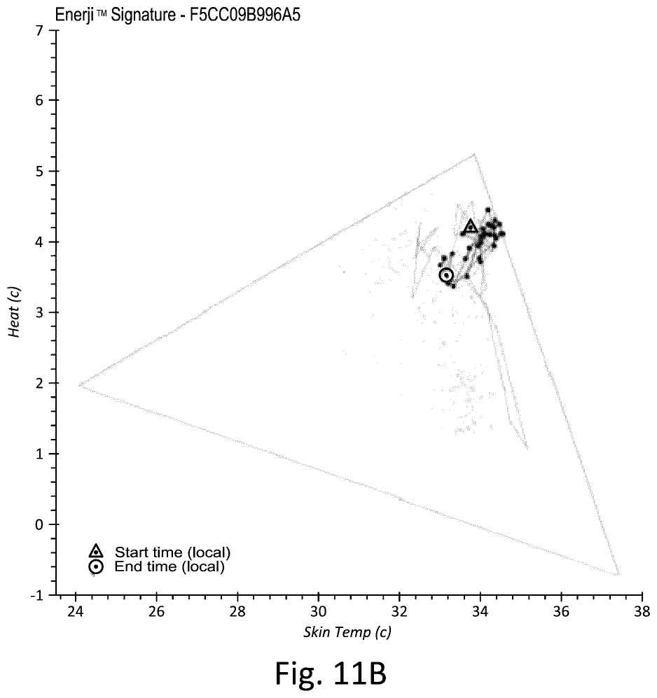

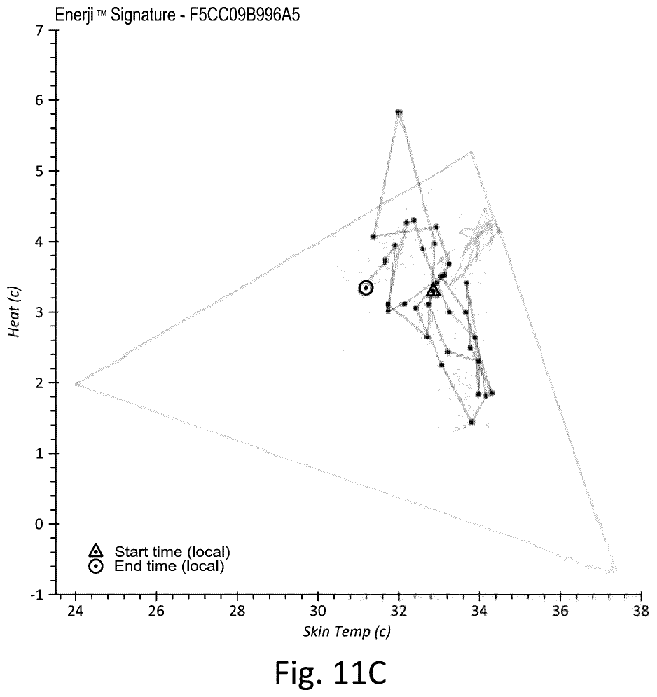

FIGS. 11A, 11B, 11C illustrate an example embodiment of recognizable features detected by the system from sleep sequences in a subject that is considered otherwise unhealthy.

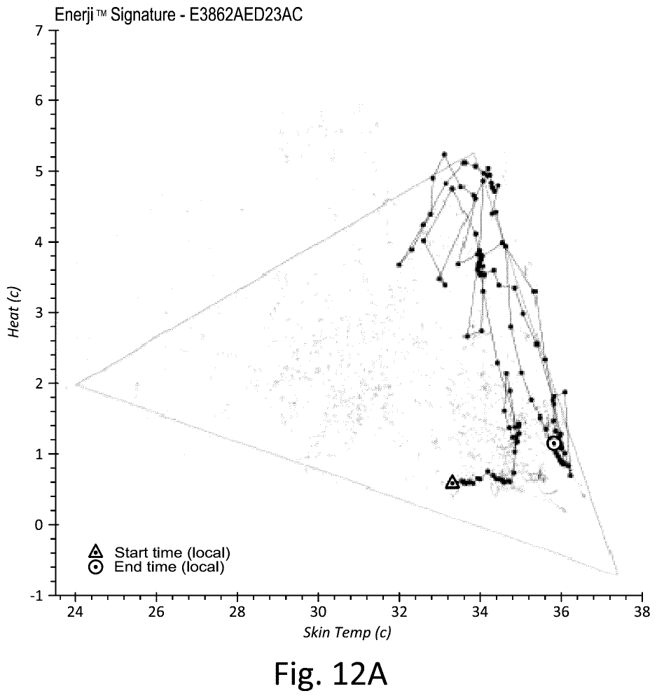

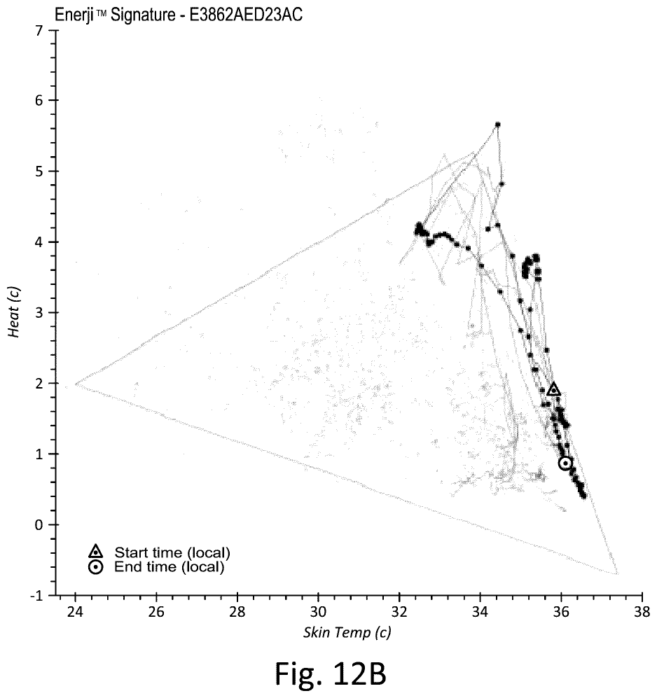

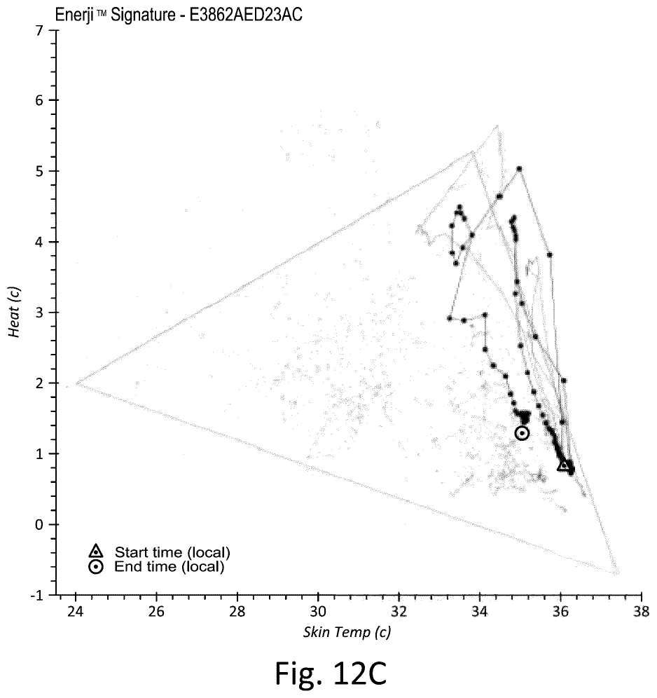

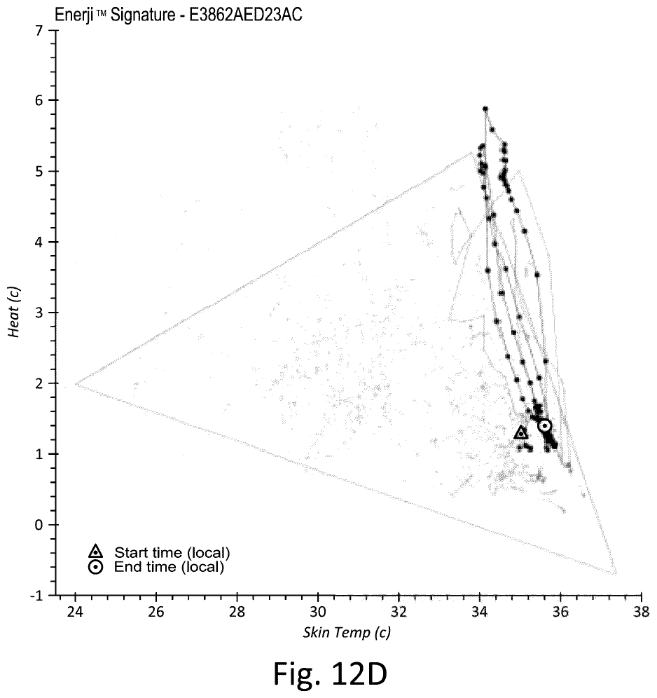

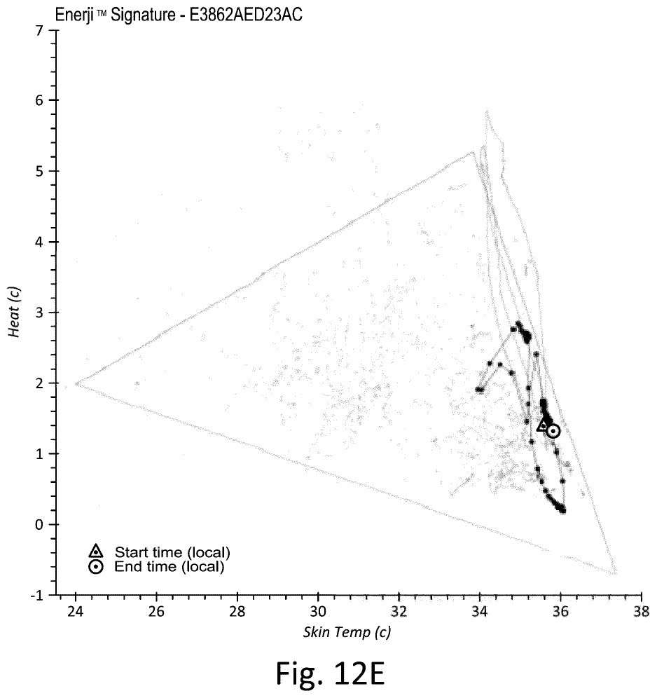

FIGS. 12A, 12B, 12C, 12D, 12E illustrate an example embodiment of recognizable features detected by the system from sleep sequences in a subject that is considered otherwise unhealthy.

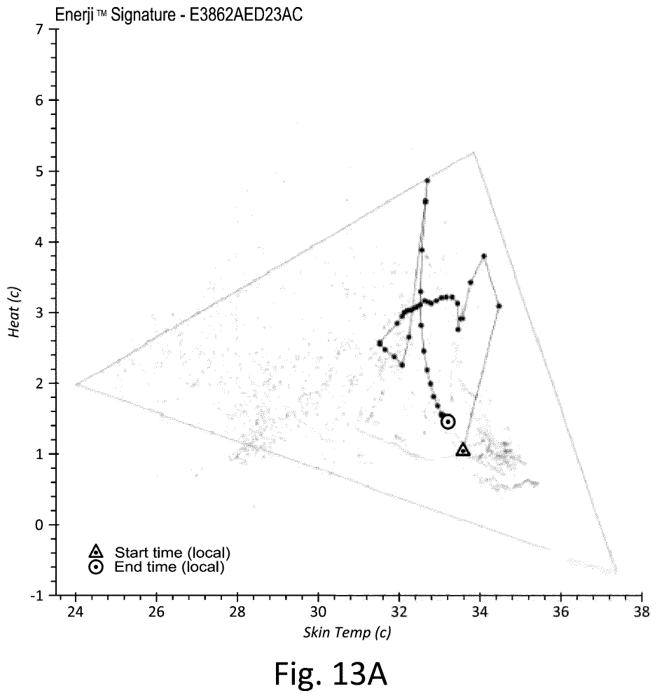

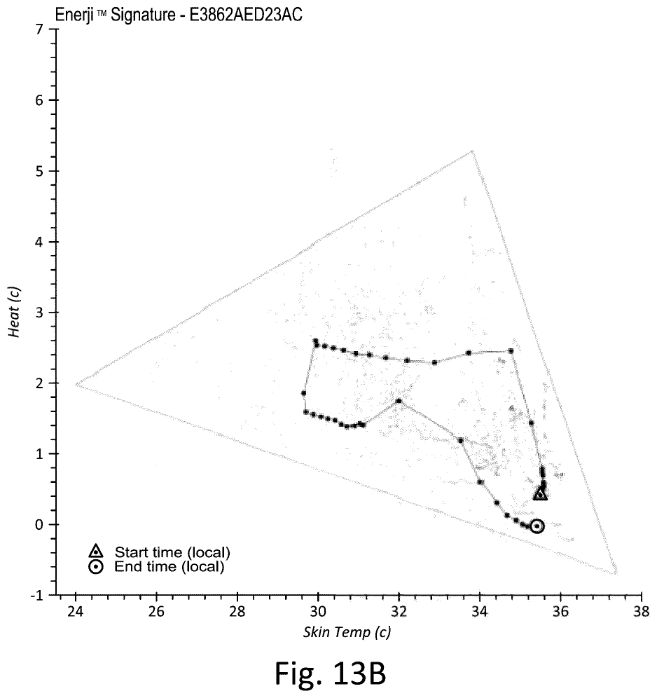

FIGS. 13A and 13B illustrate an example embodiment of recognizable features detected by the system from menopausal hot flash in a subject.

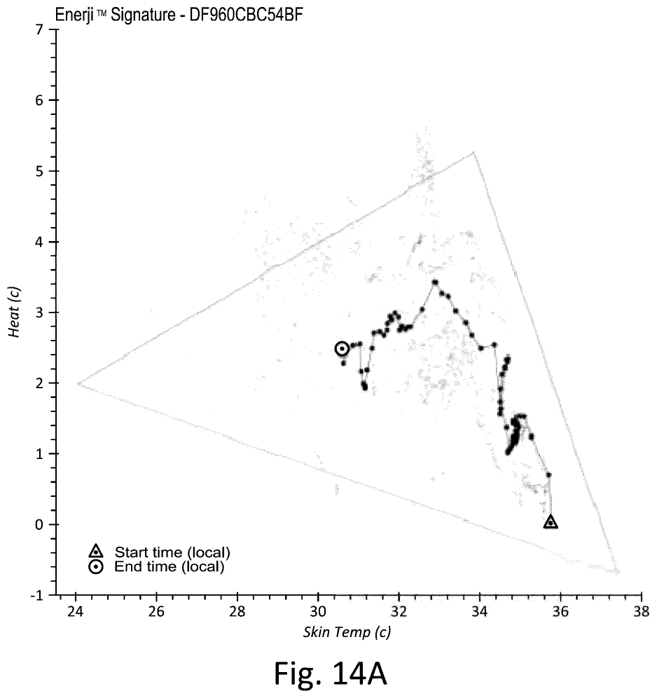

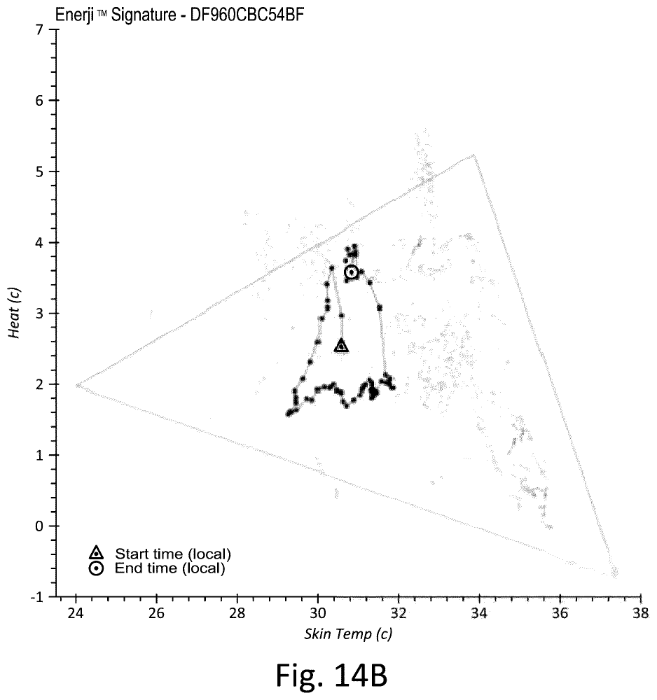

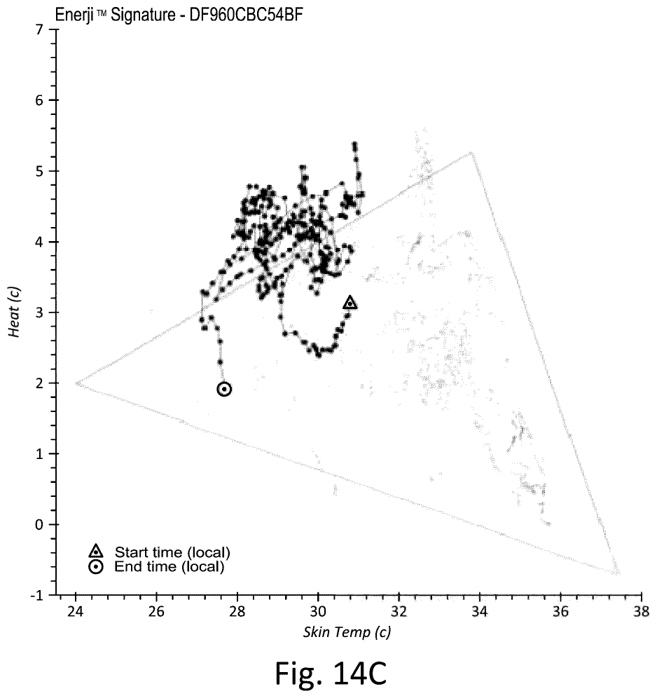

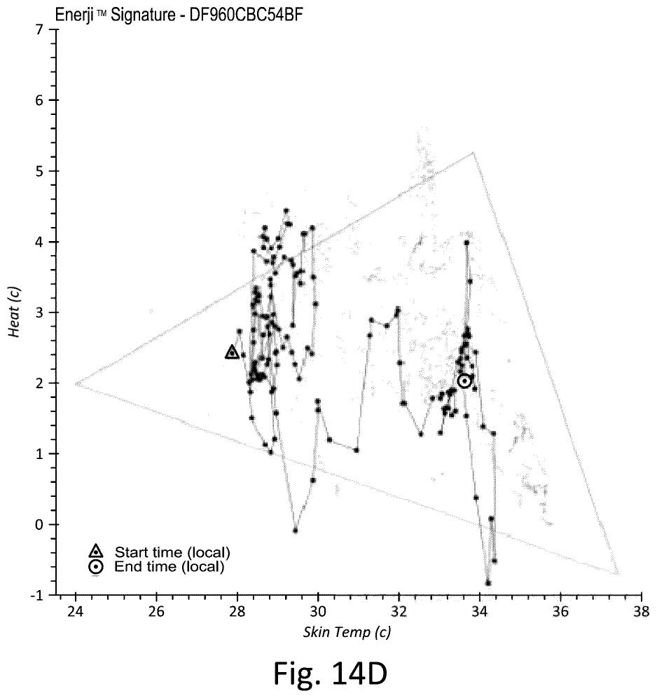

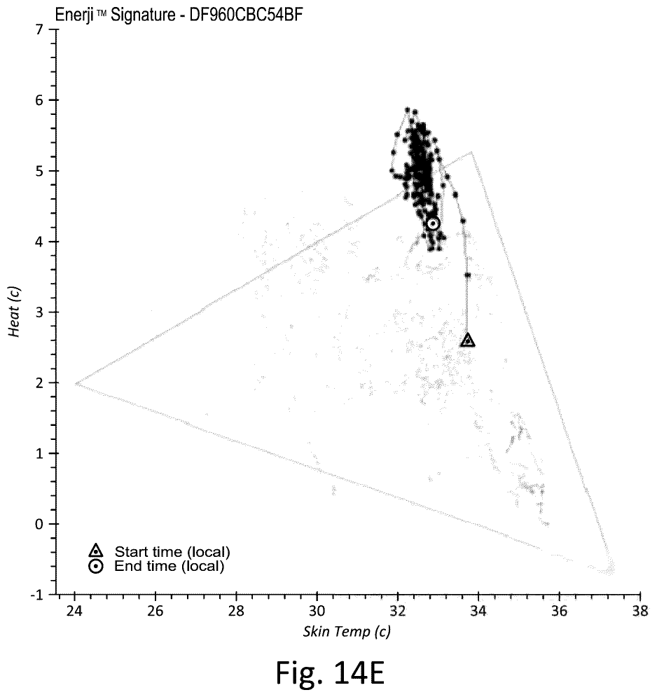

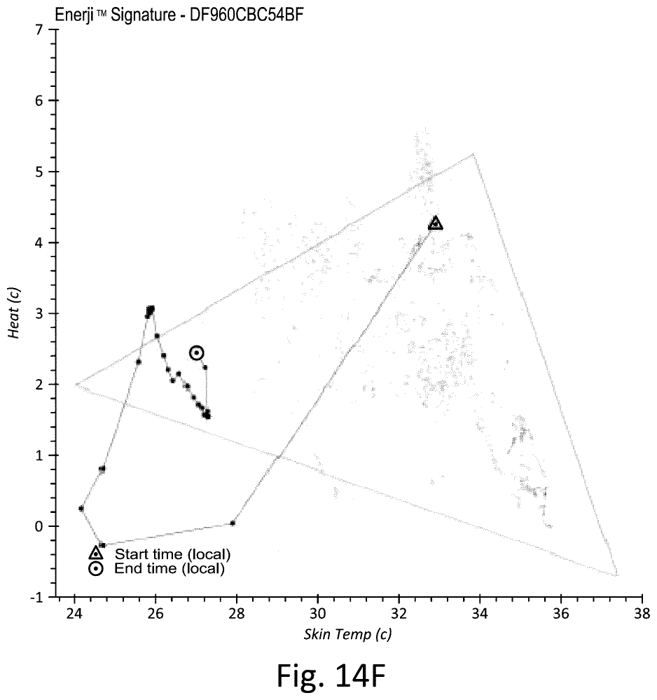

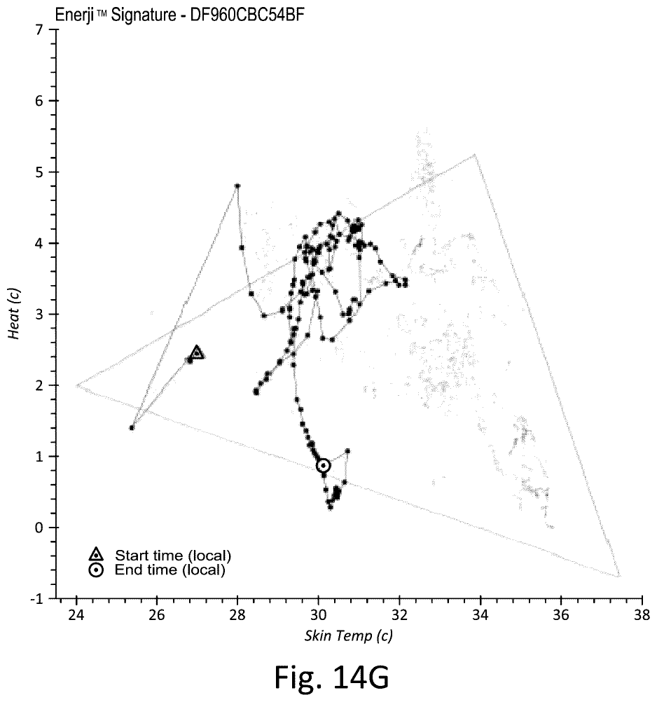

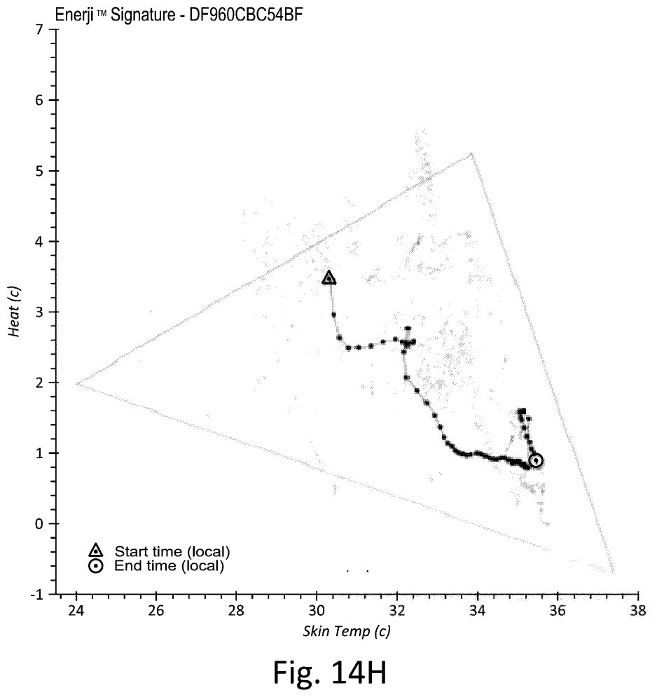

FIGS. 14A, 14B, 14C, 14D, 14E, 14F, 14G, 14H illustrate an example embodiment of recognizable features detected by the system from physical exertion a subject. FIG. 14A illustrates an example embodiment of recognizable features detected by the system when the subject is aroused by sleep. FIG. 14B illustrates an example embodiment of recognizable features detected by the system when the subject is driving to the trailhead. FIG. 14C illustrates an example embodiment of recognizable features detected by the system when the subject is level hiking leading to steep ascent. FIG. 14D illustrates an example embodiment of recognizable features detected by the system when the subject is on summit and resting. FIG. 14E illustrates an example embodiment of recognizable features detected by the system when the subject is on a steep descent. FIG. 14F illustrates an example embodiment of recognizable features detected by the system when the subject is cooling down in the river. FIG. 14G illustrates an example embodiment of recognizable features detected by the system when the subject has finished the hike. FIG. 14H illustrates an example embodiment of recognizable features detected by the system when the subject has fallen asleep.

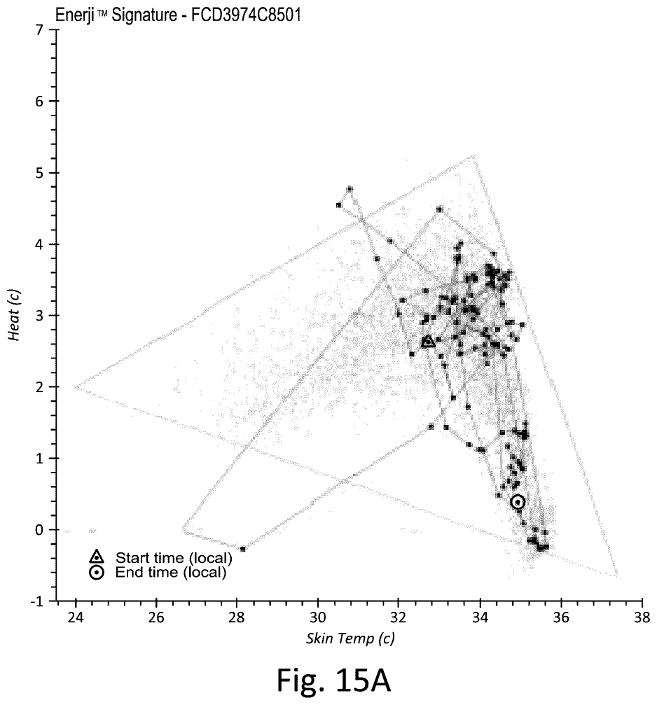

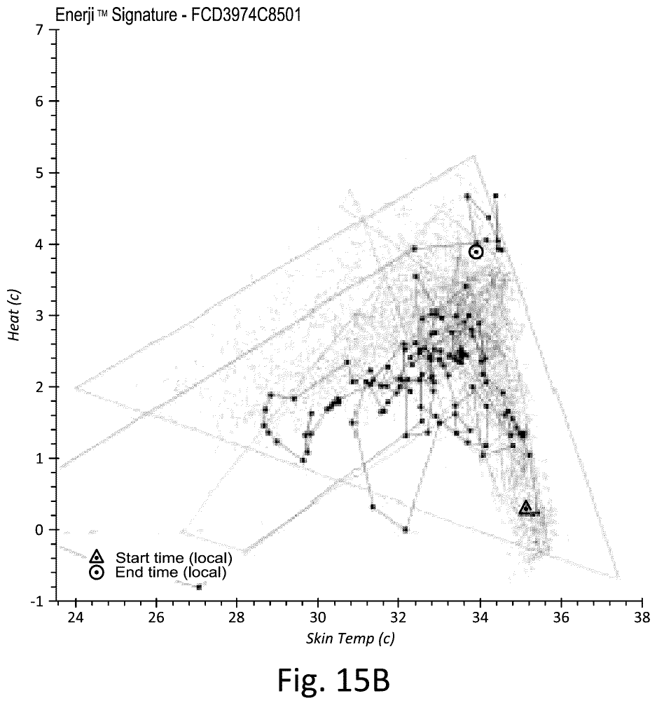

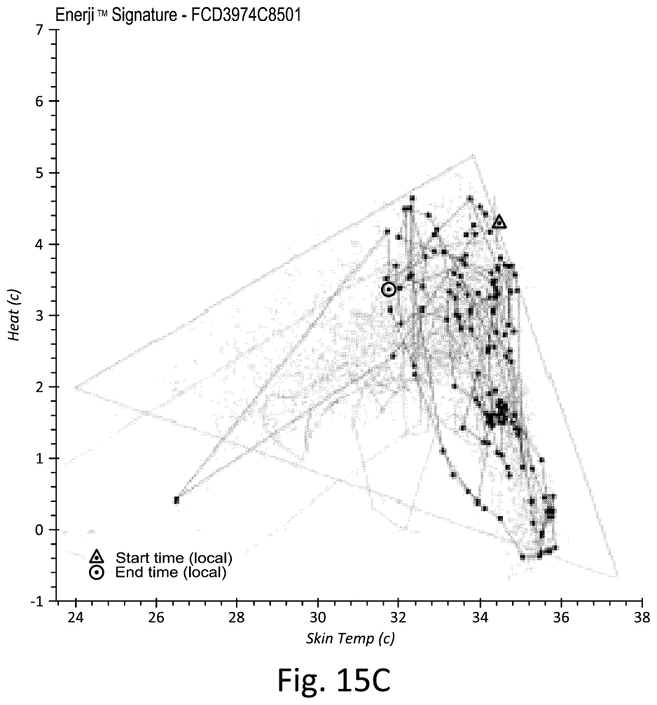

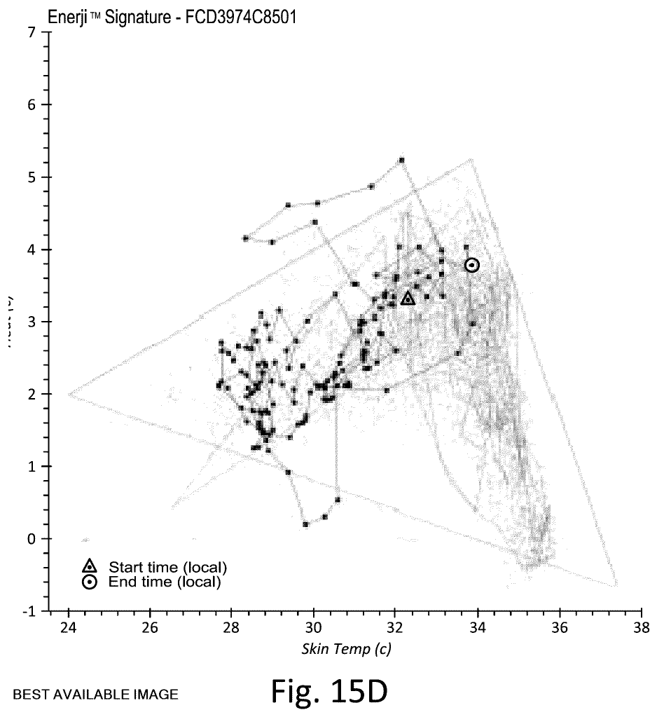

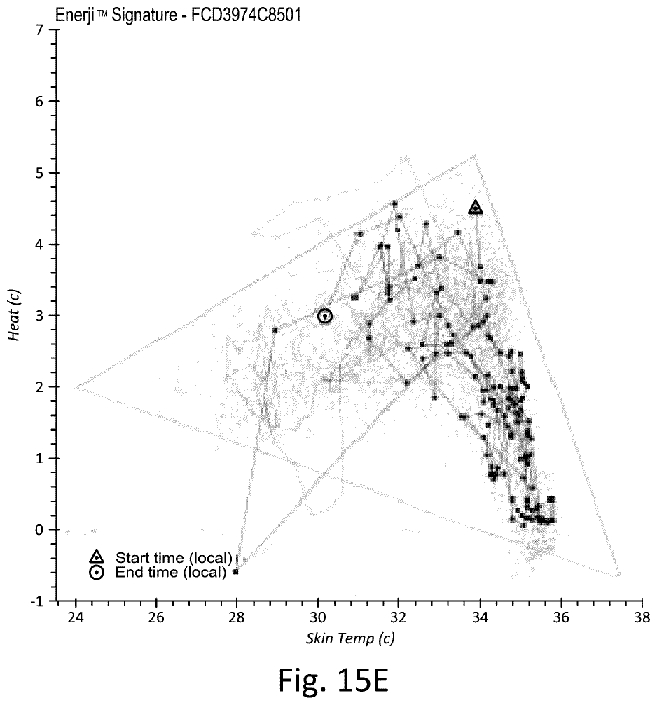

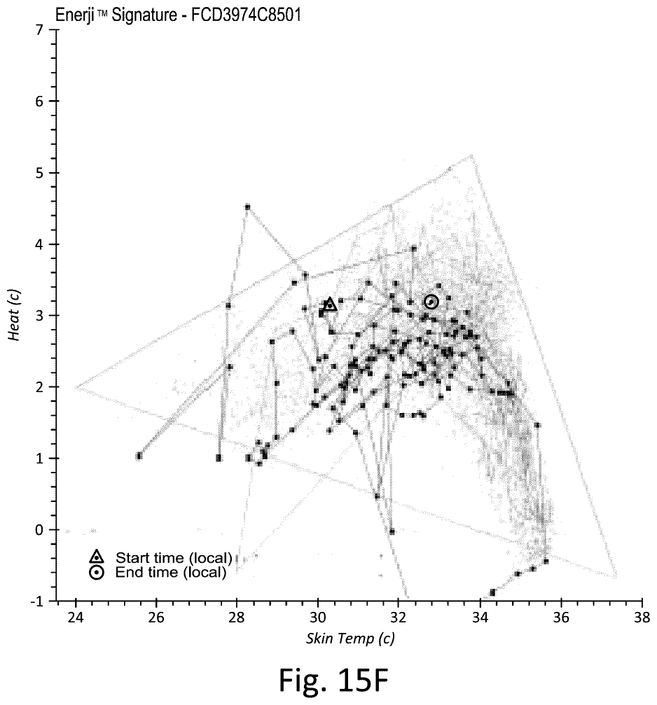

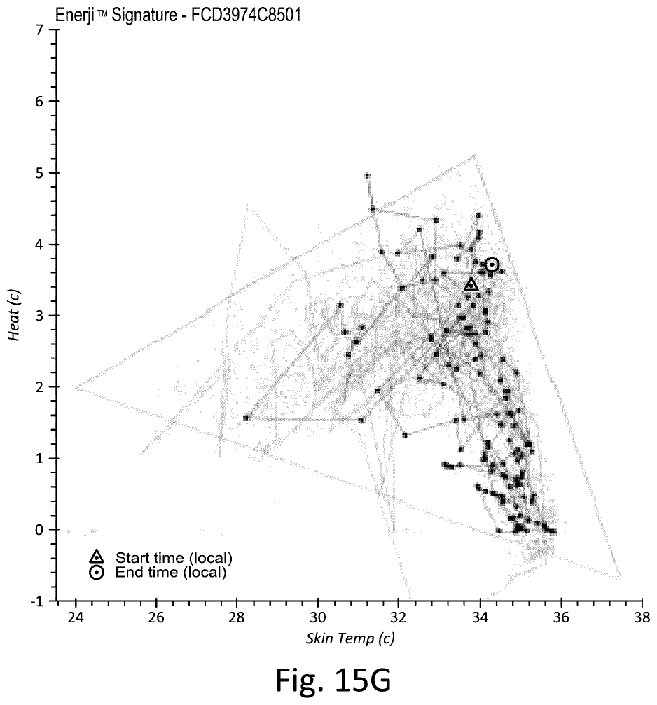

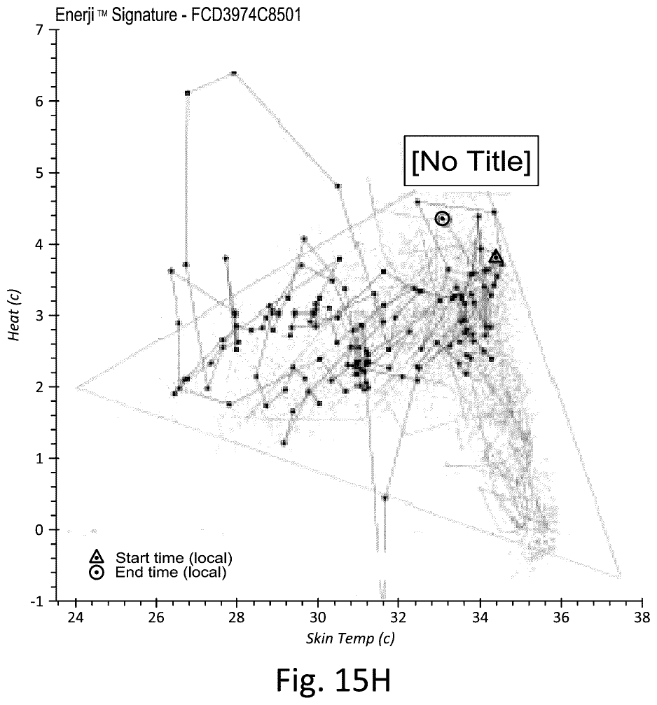

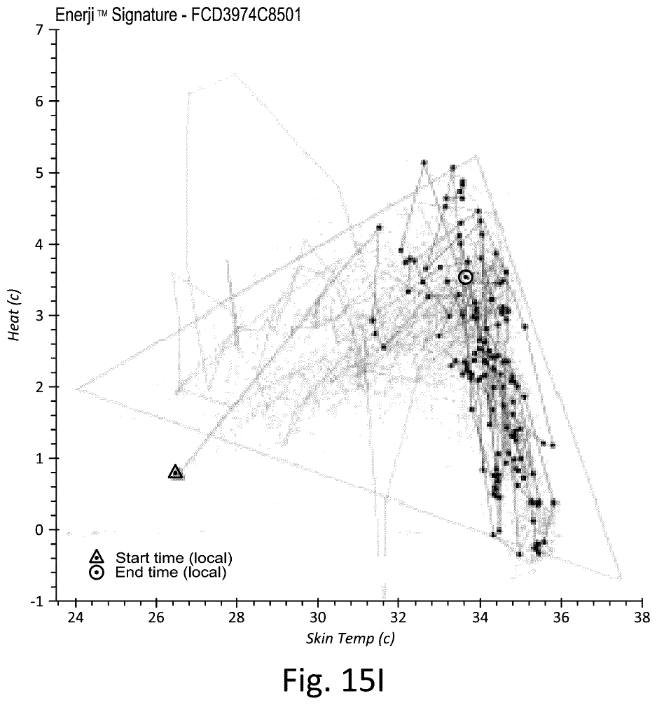

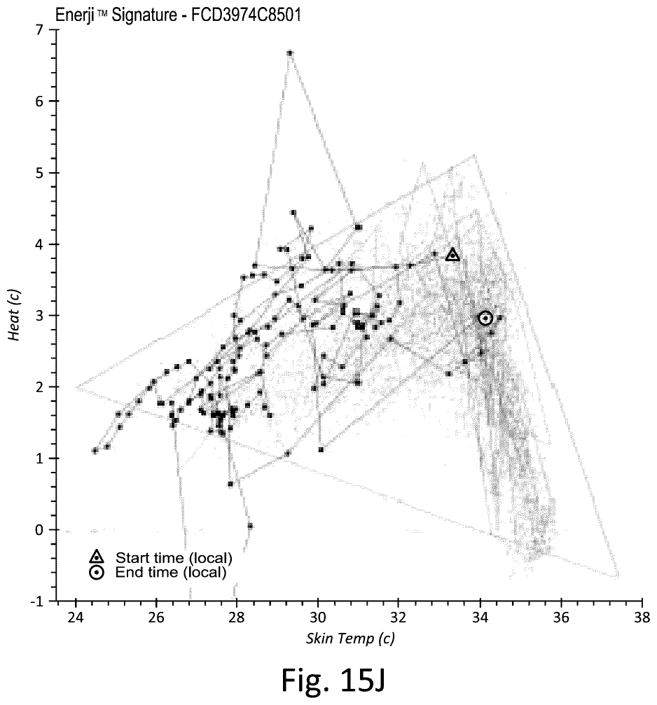

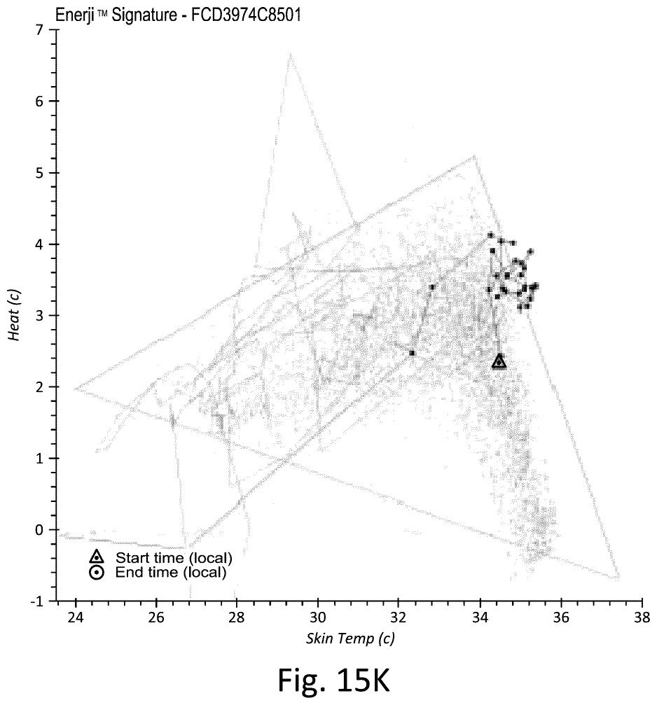

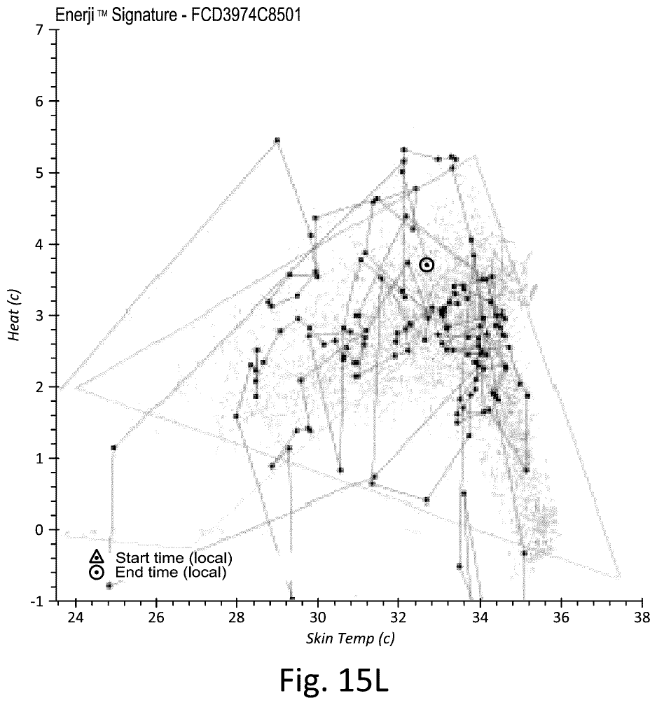

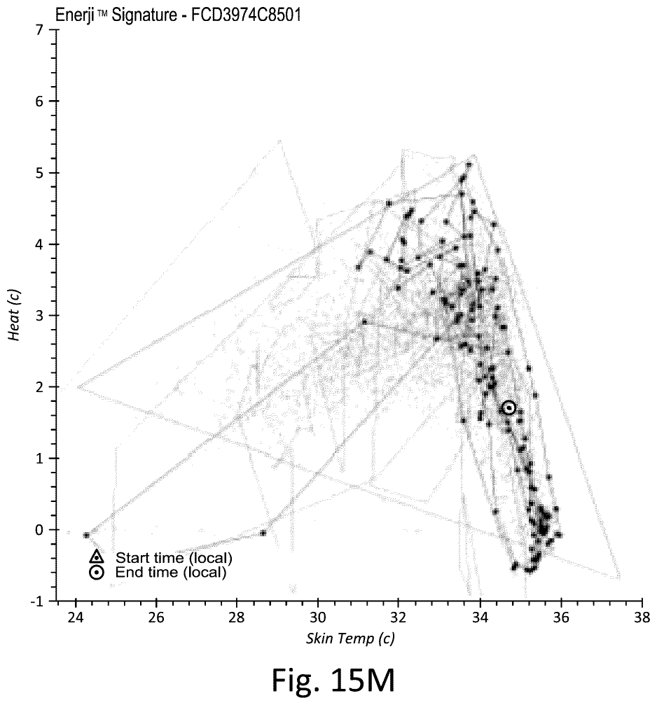

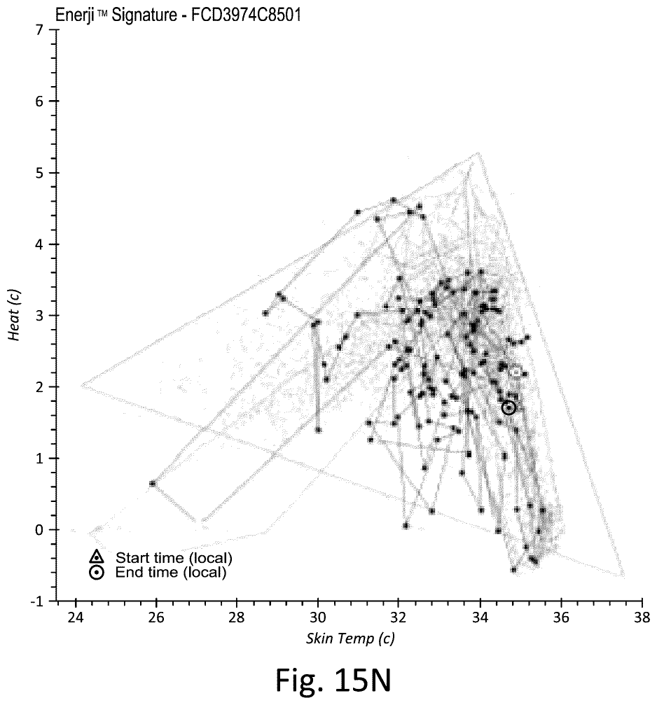

FIGS. 15A, 15B, 15C, 15D, 15E, 15F, 15G, 15H, 151, 15J, 15K, 15L, 15M, 15N illustrate an example embodiment of recognizable features detected by the system for an 8 hour period over days in a subject that is otherwise considered healthy.

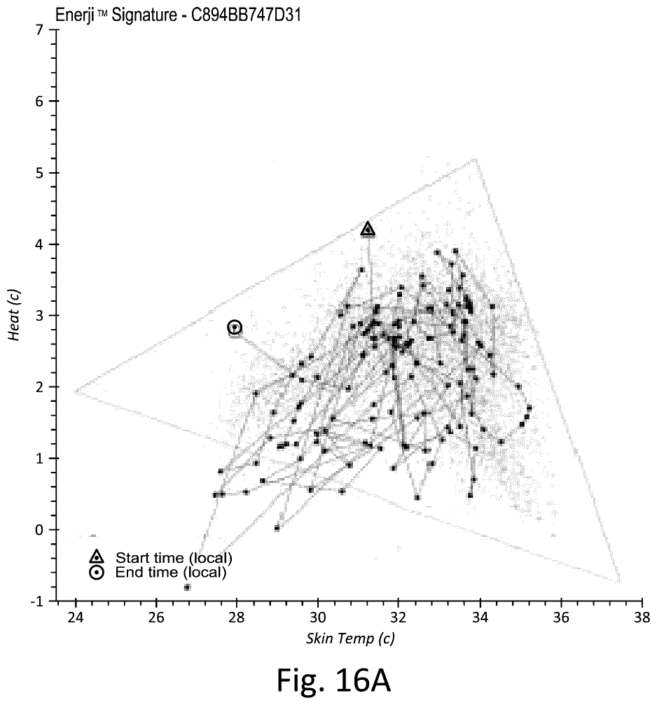

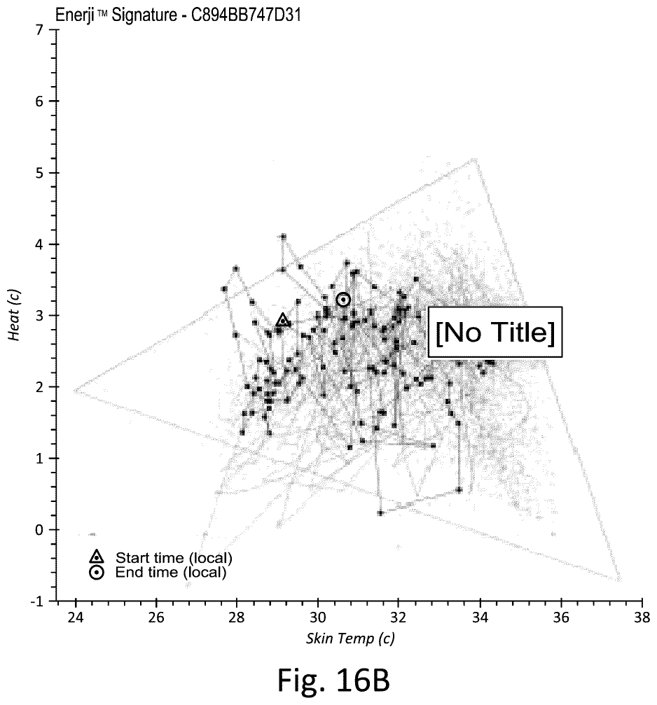

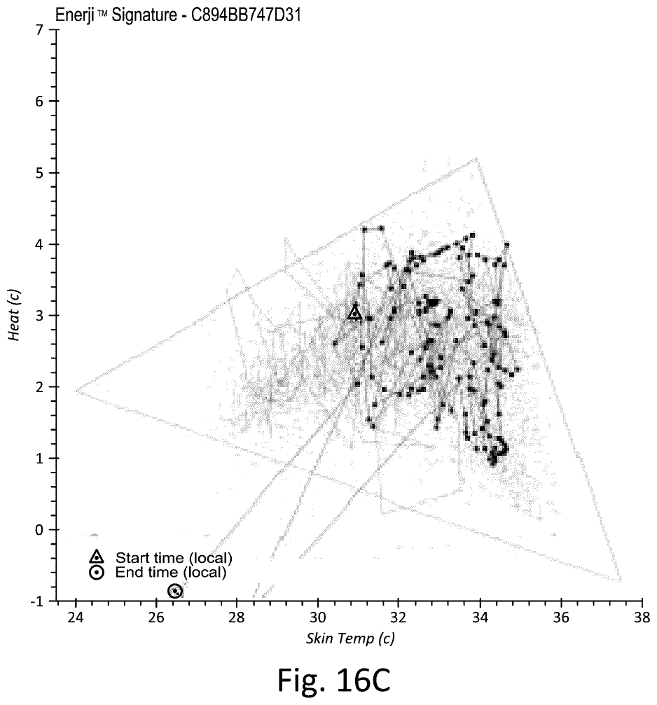

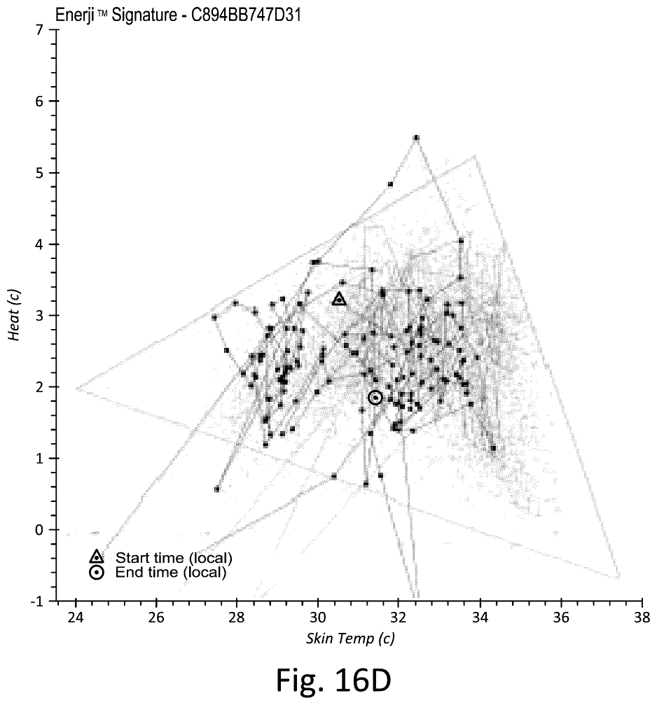

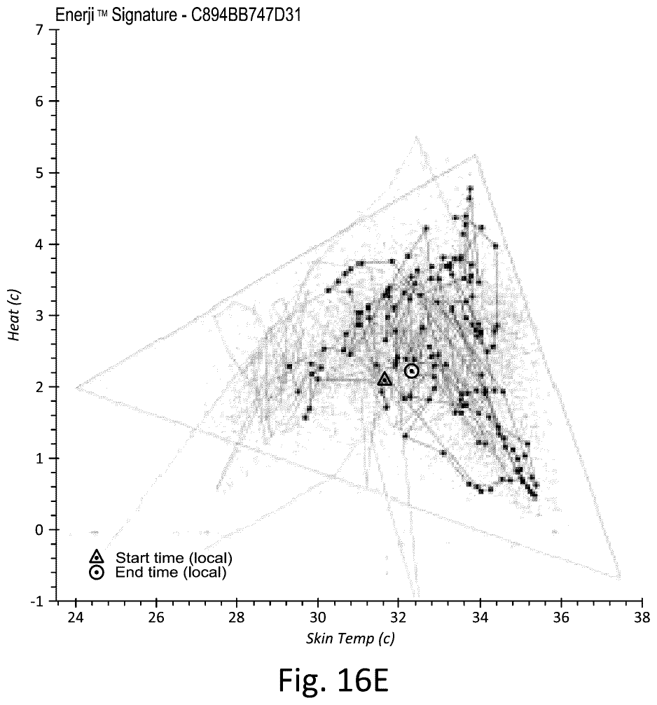

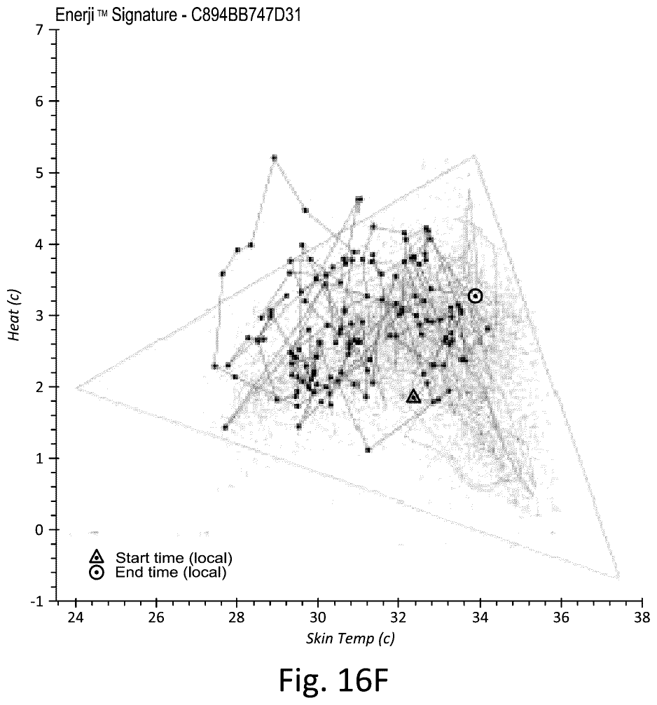

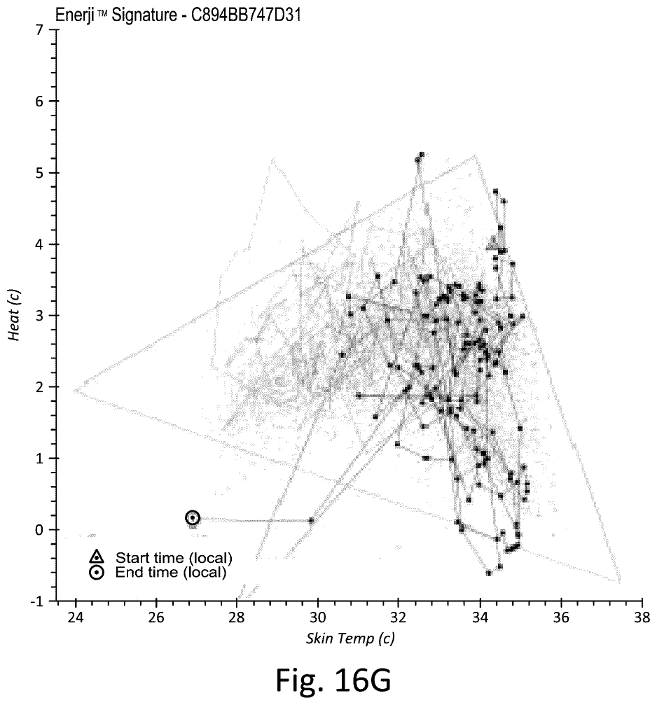

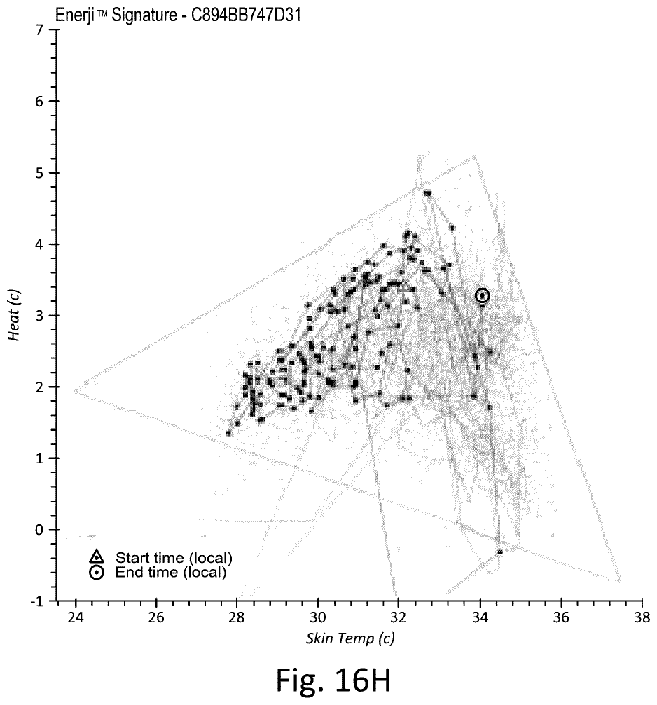

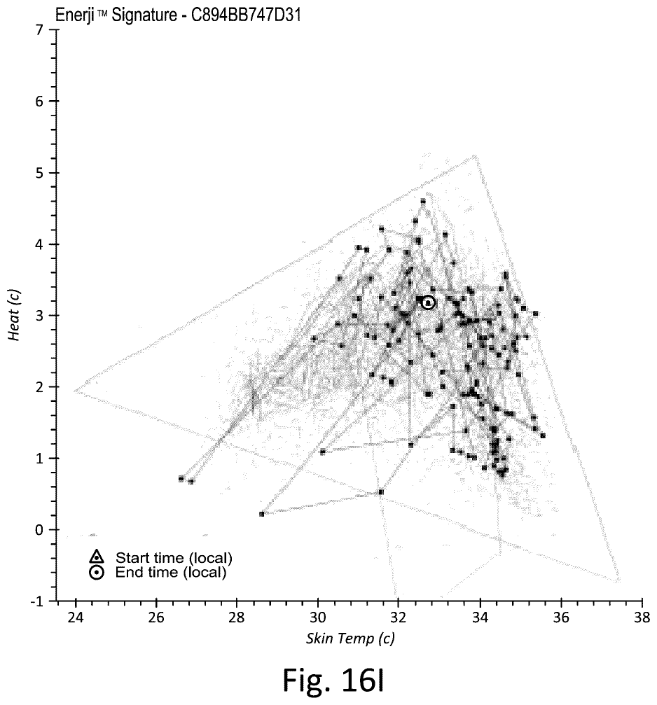

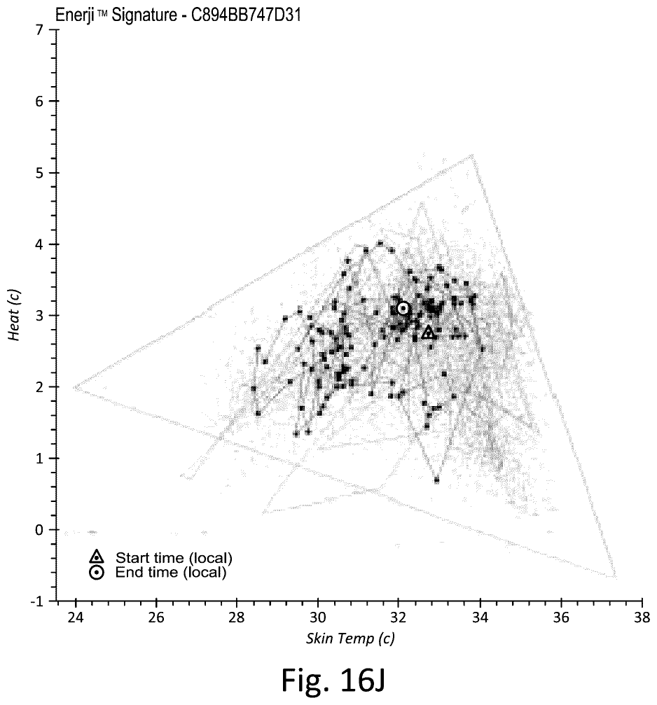

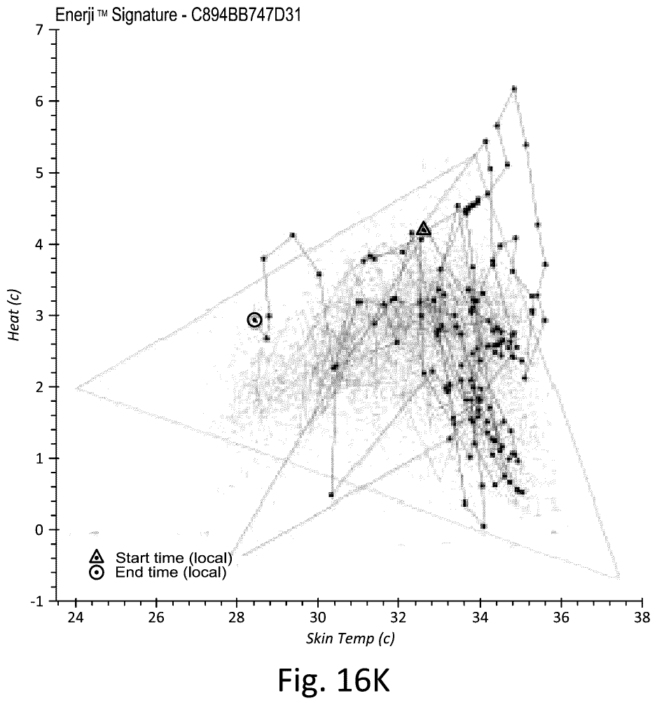

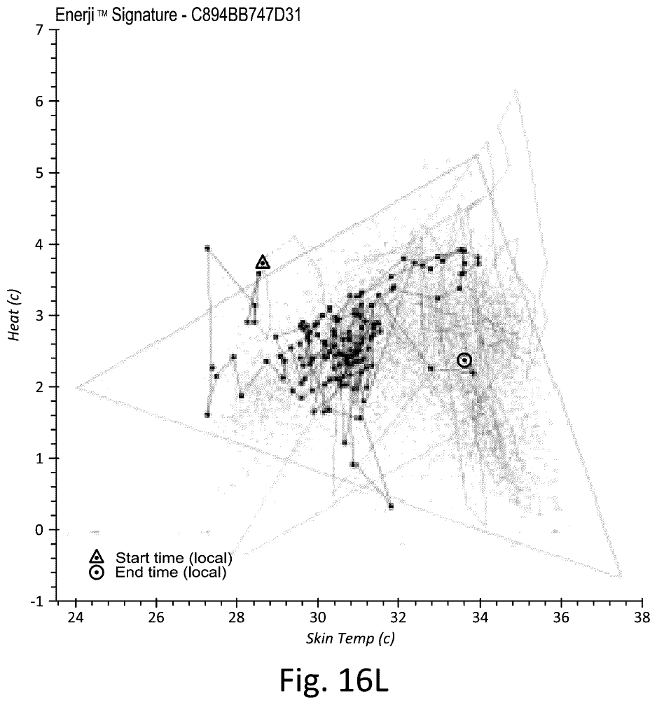

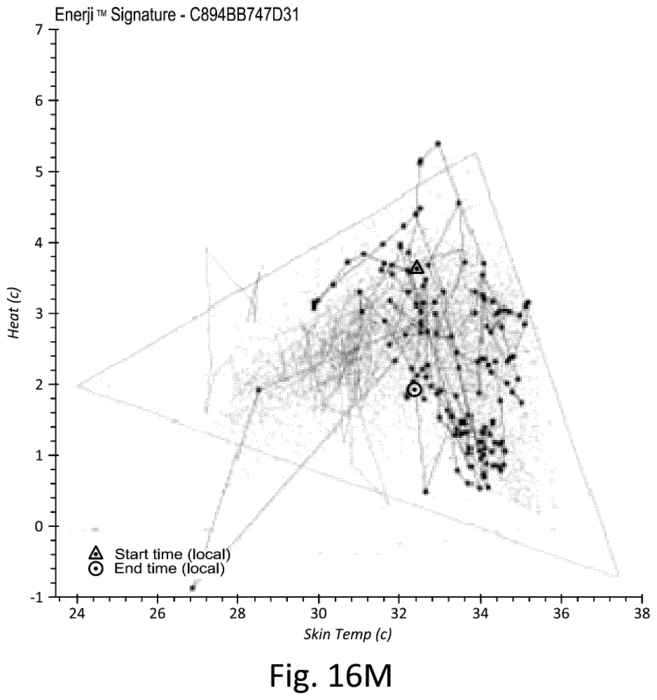

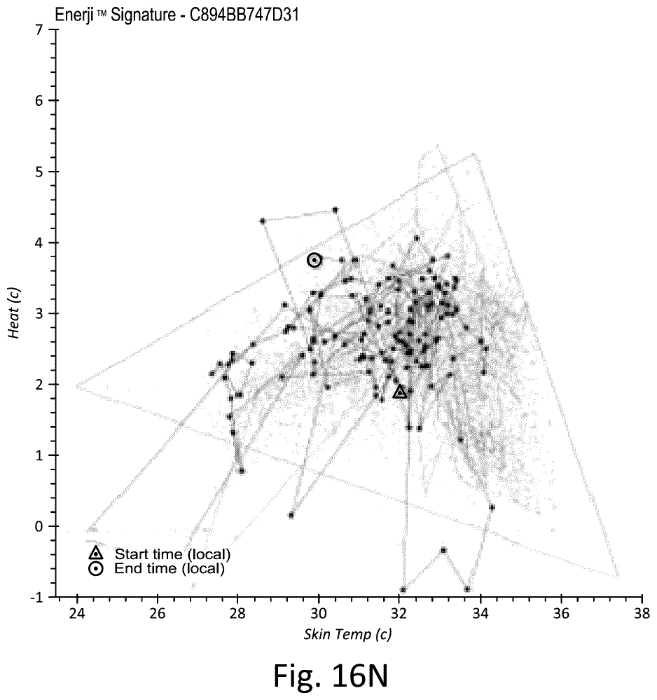

FIGS. 16A, 16B, 15C, 16D, 16E, 16F, 16G, 16H, 161, 16J, 16K, 16L, 16M, 16N illustrate an example embodiment of recognizable features detected by the system for an 8 hour period over days in a subject that is otherwise considered healthy.

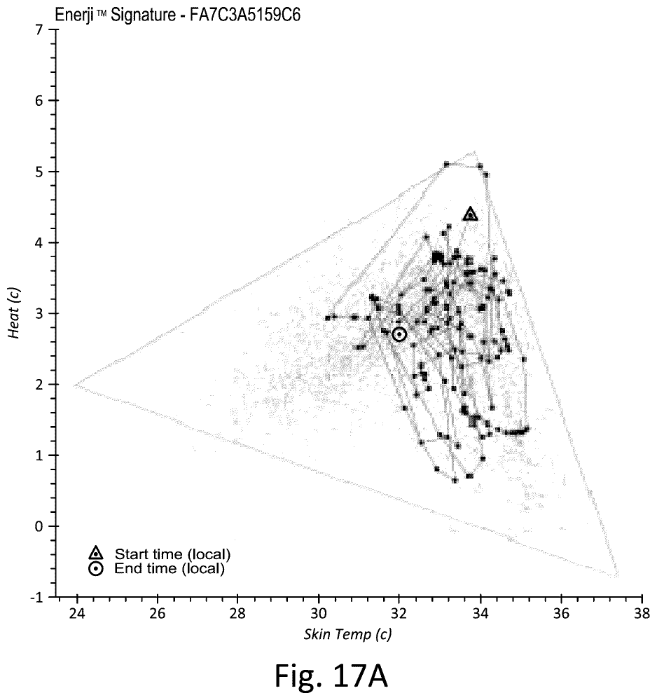

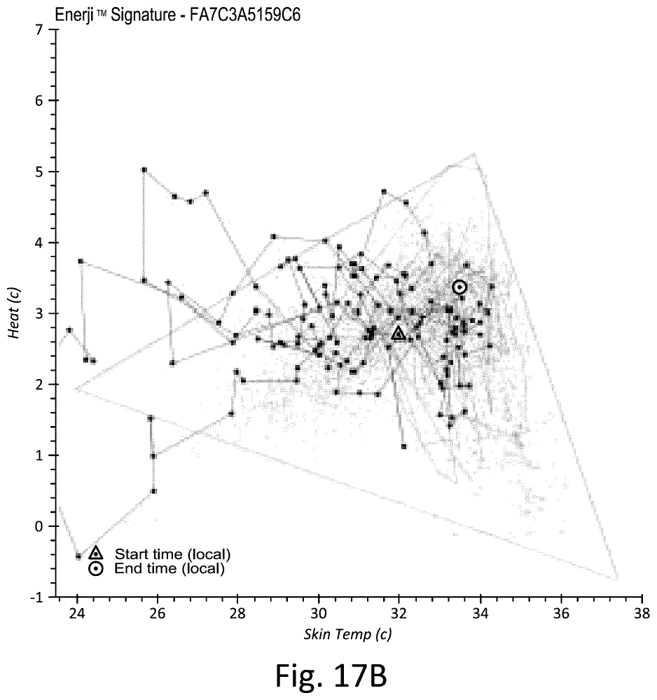

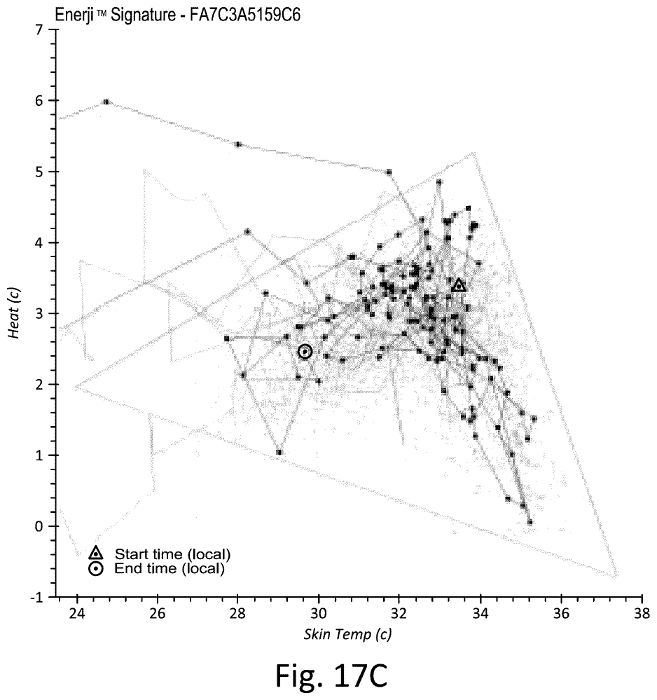

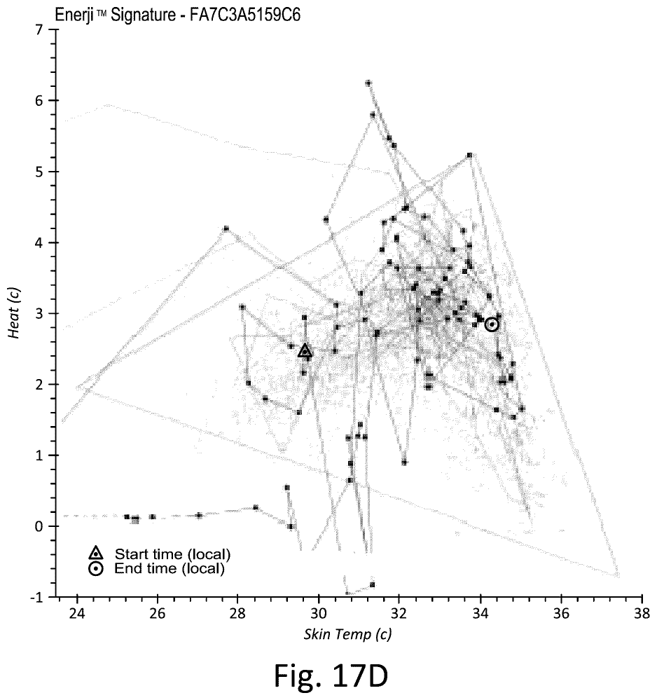

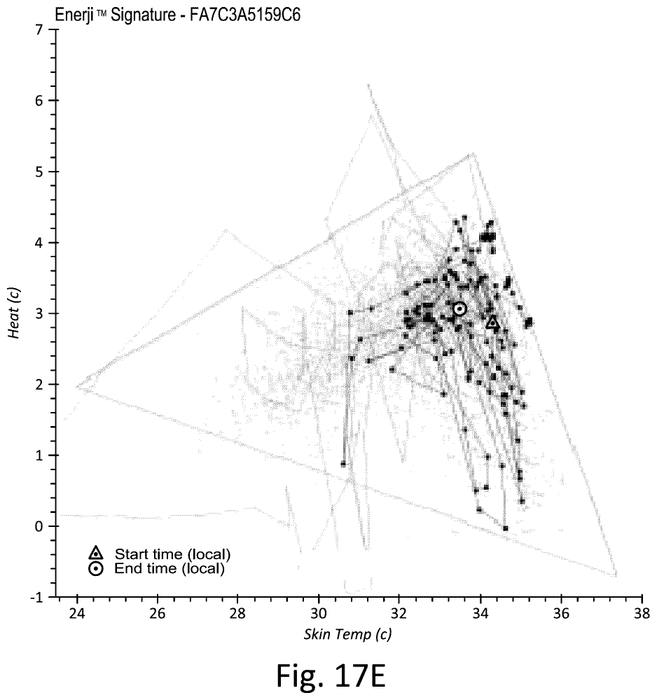

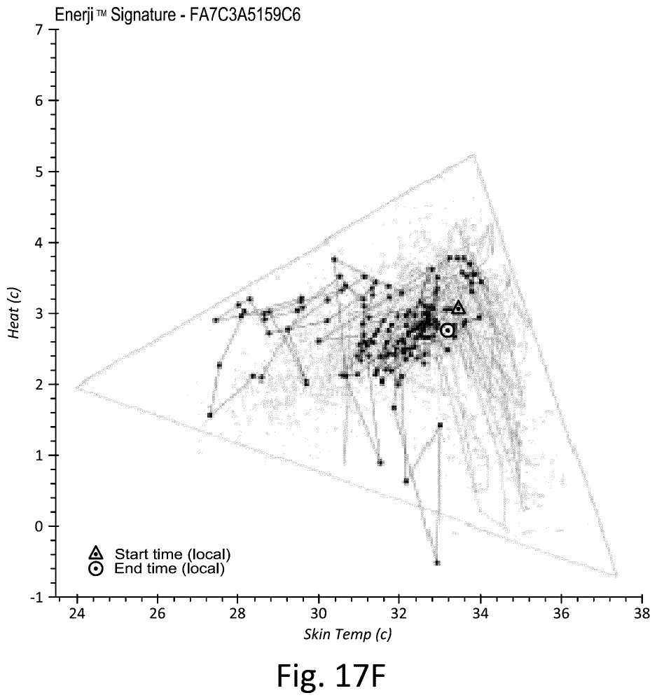

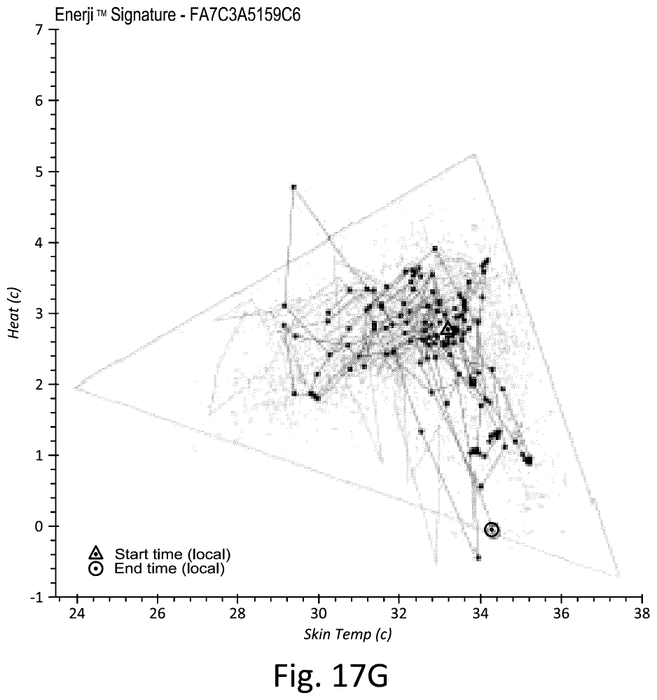

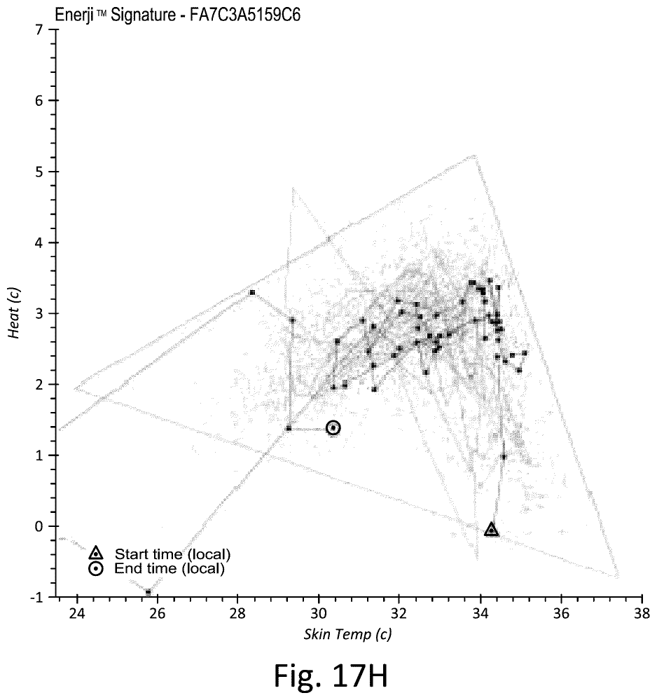

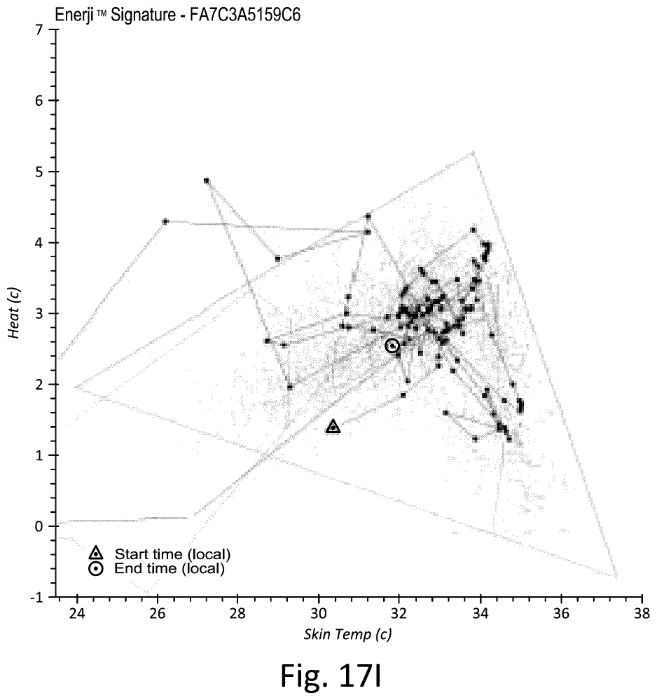

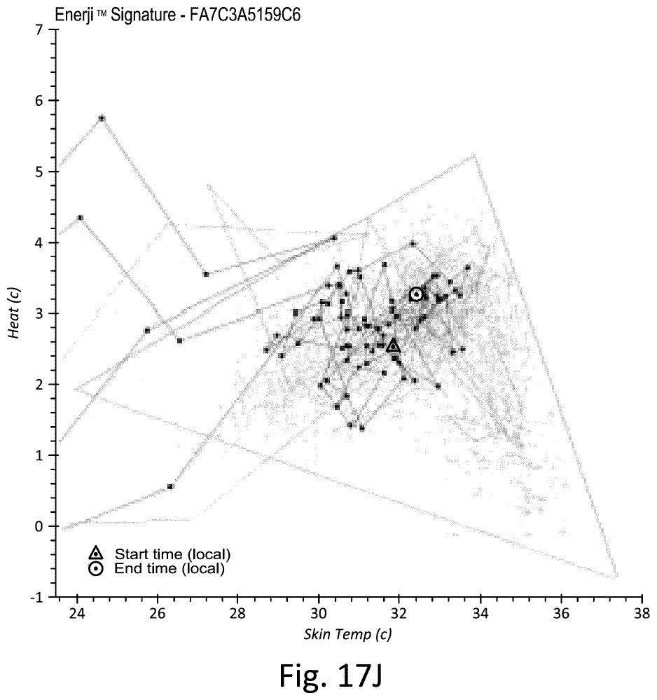

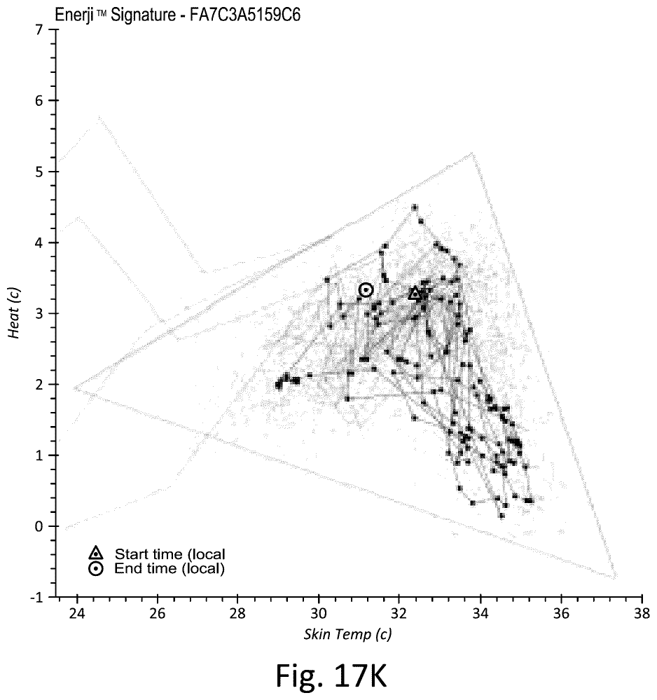

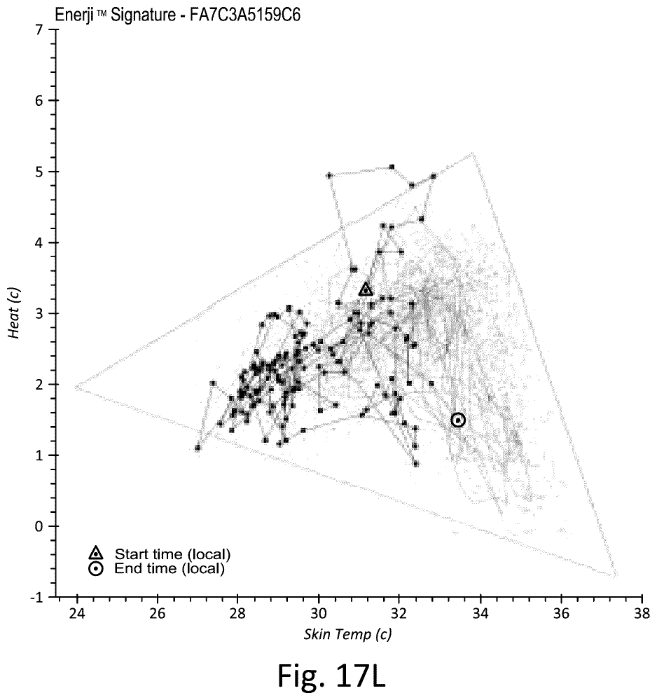

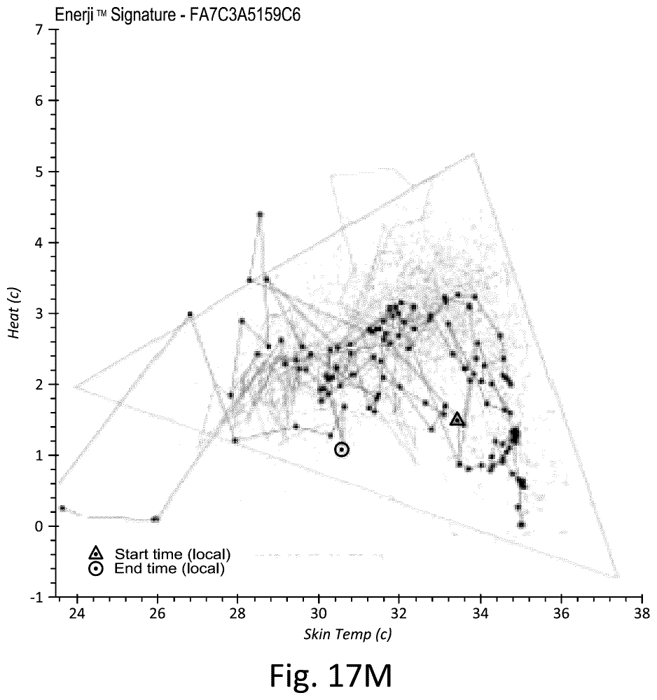

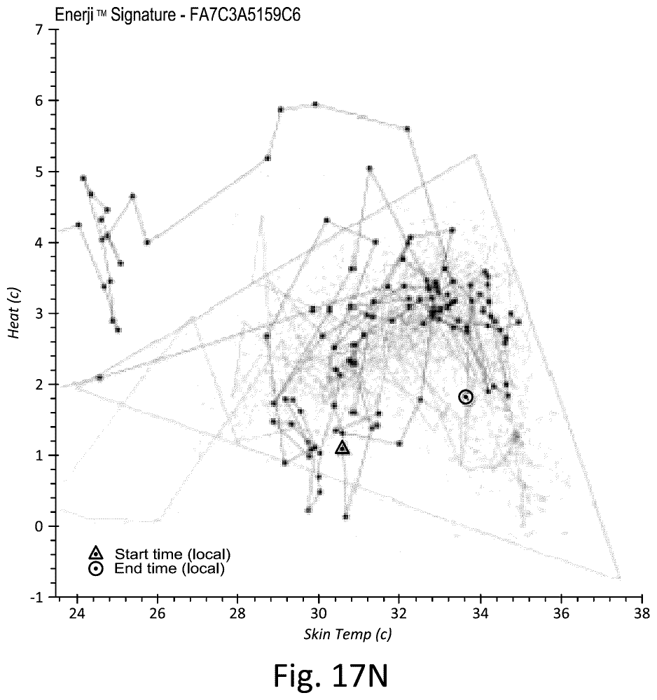

FIGS. 17A, 17B, 17C, 17D, 17E, 17F, 17G, 17H, 171, 17J, 17K, 17L, 17M, 17N illustrate an example embodiment of recognizable features detected by the system for an 8 hour period over days in a subject that is otherwise considered healthy.

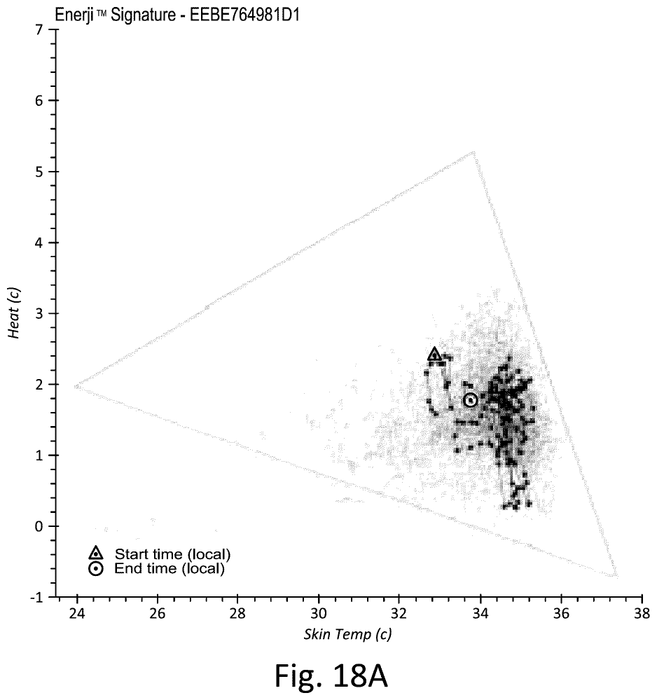

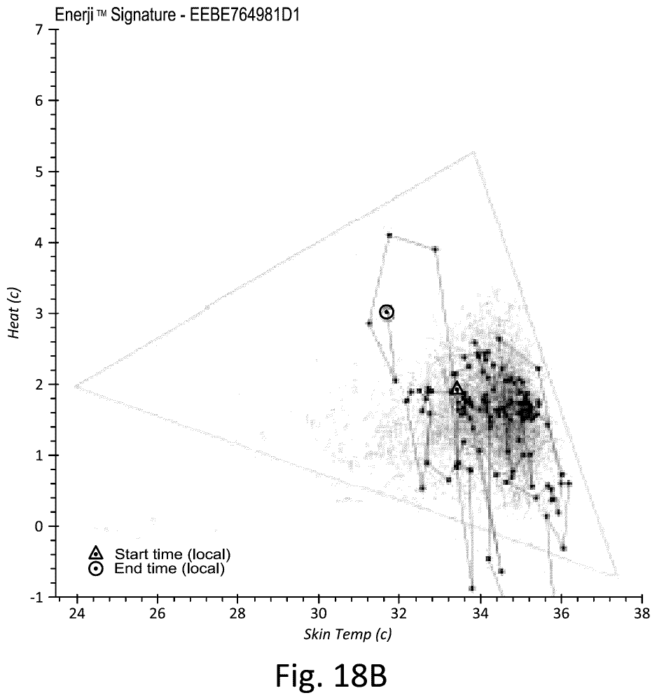

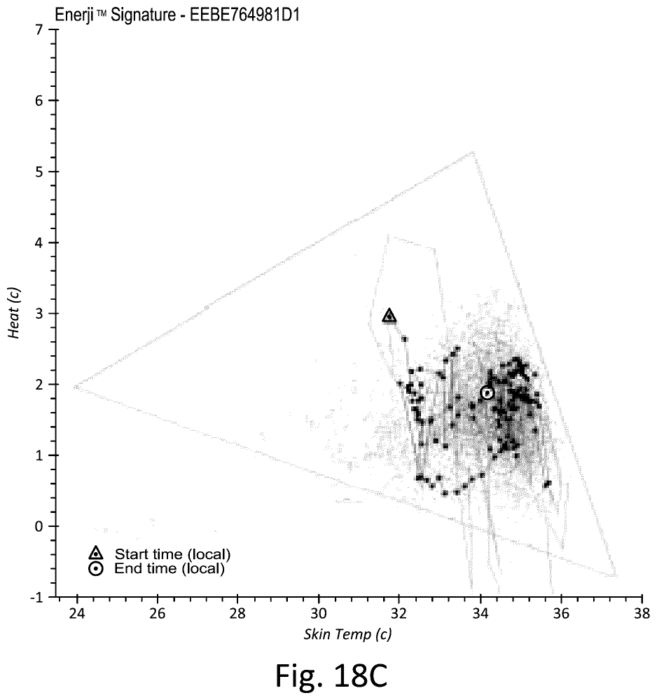

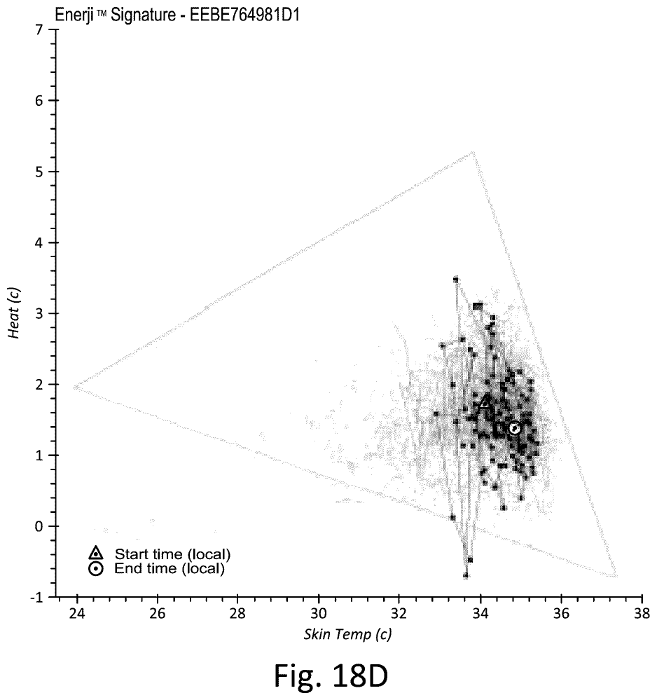

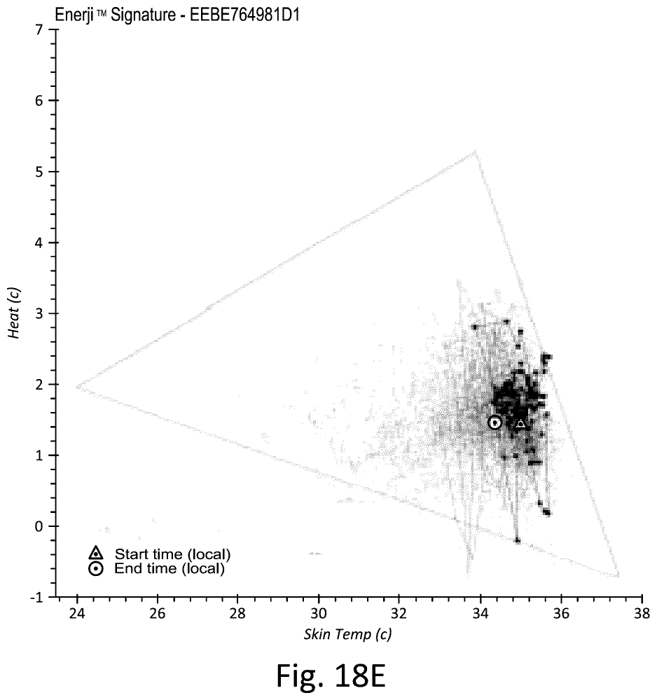

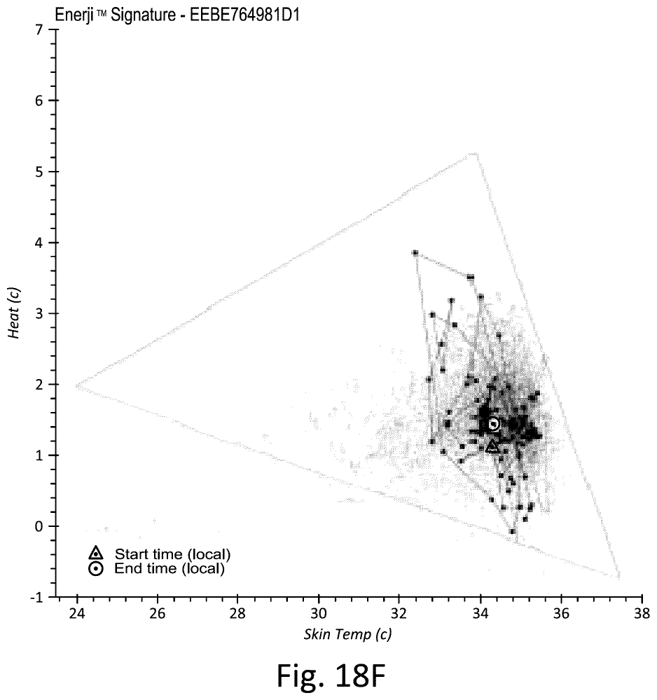

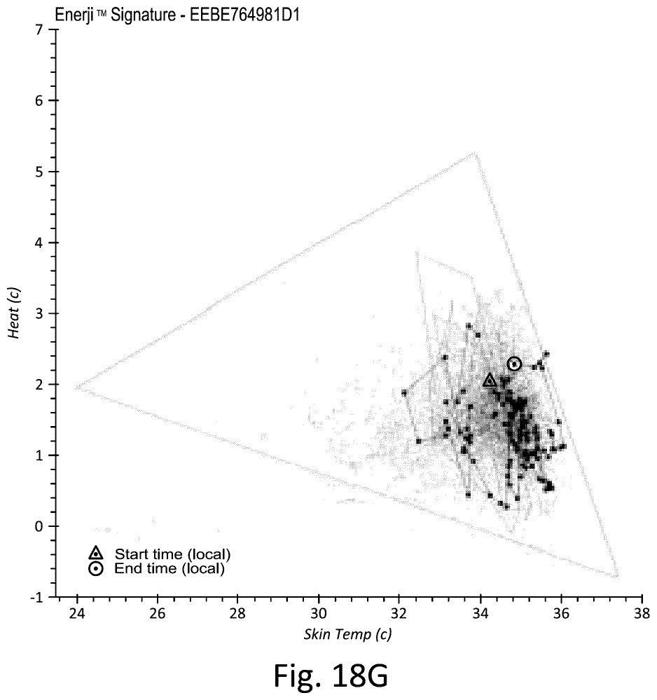

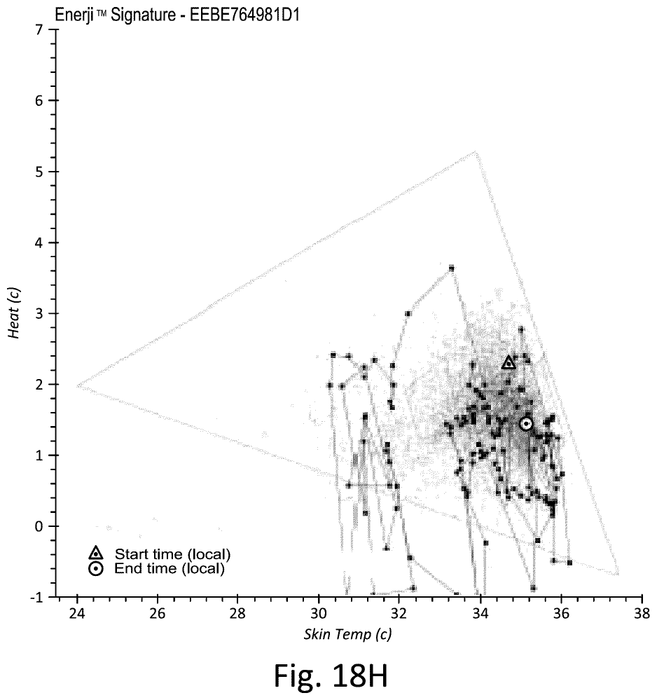

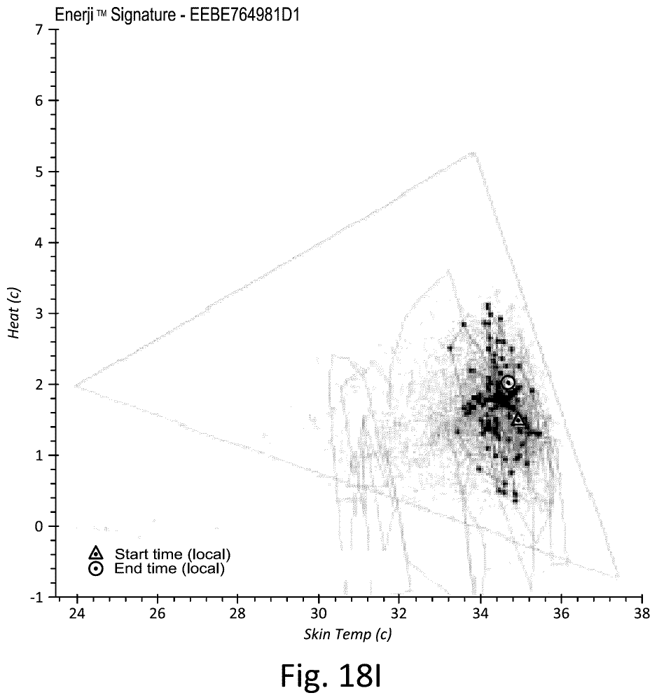

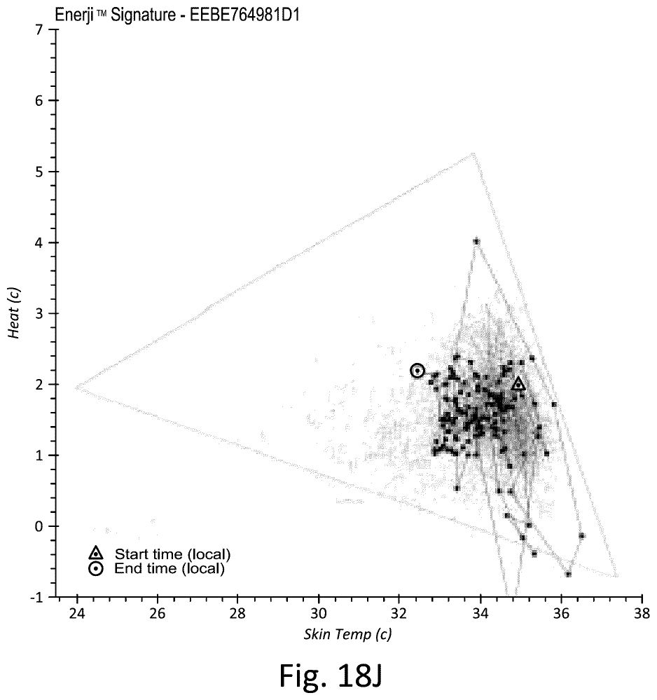

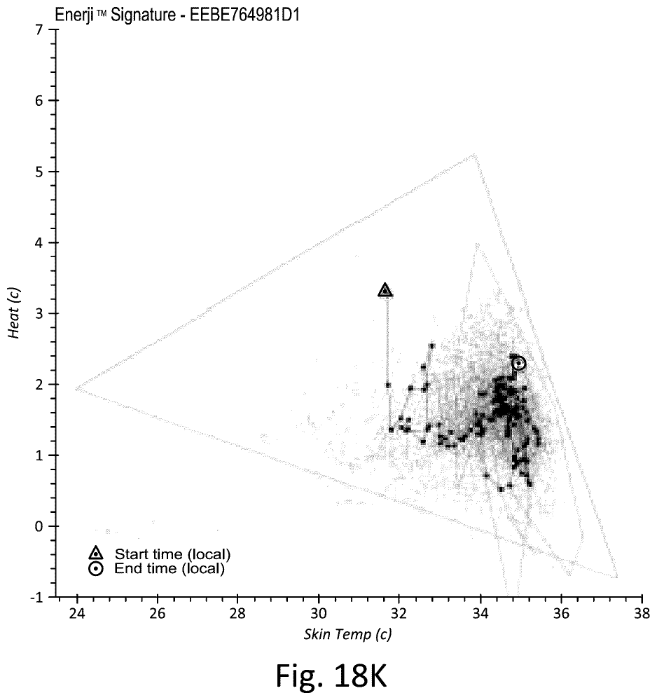

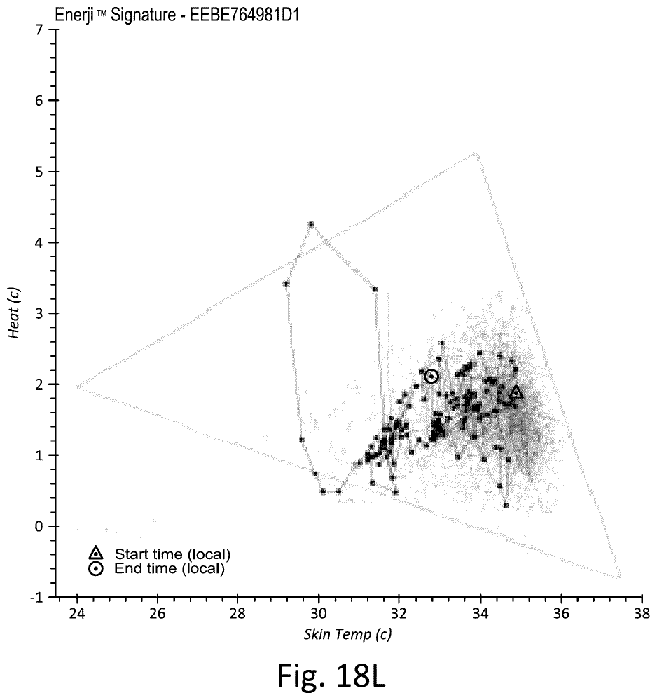

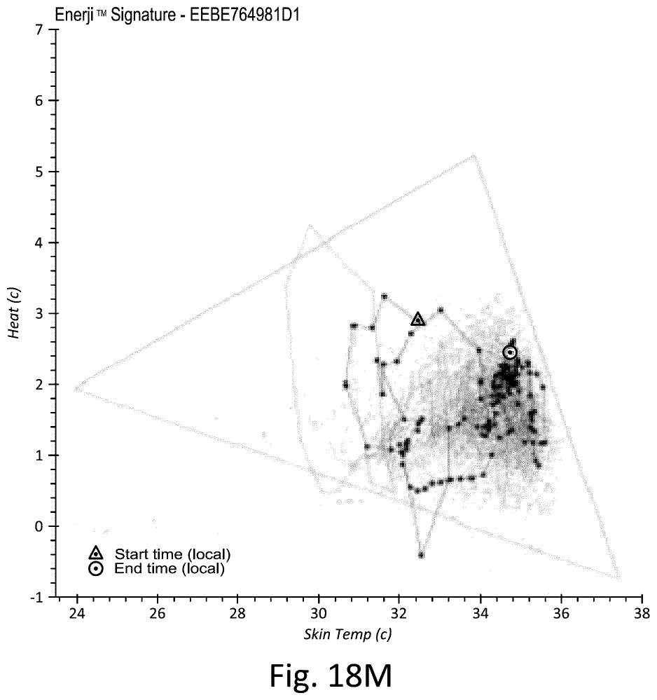

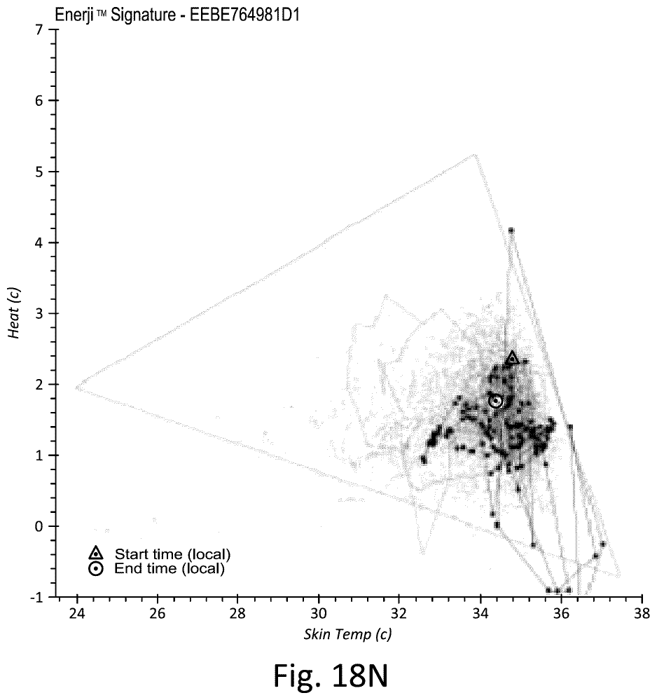

FIGS. 18A, 18B 18C, 18D, 18E, 18F, 18G, 18H, 18I, 18J, 18K, 18L, 18M, 18N illustrate an example embodiment of recognizable features detected by the system for an 8 hour period over days in a subject with chronic heart failure.

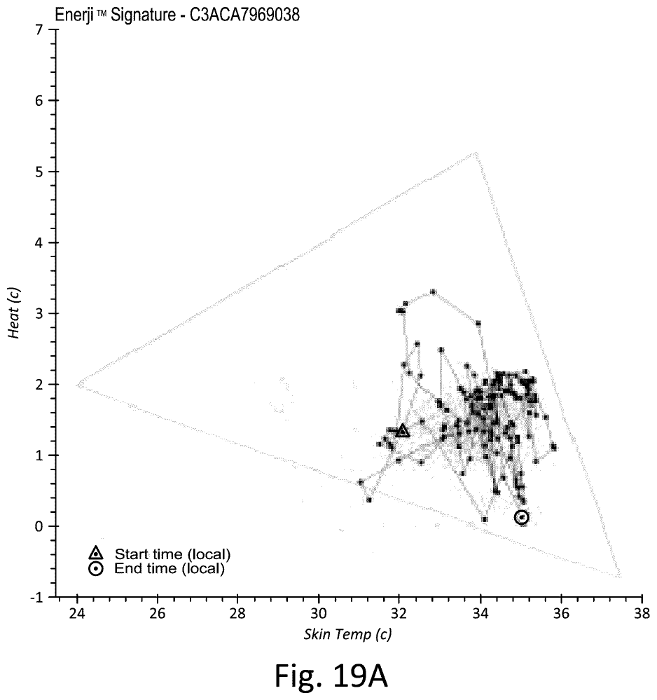

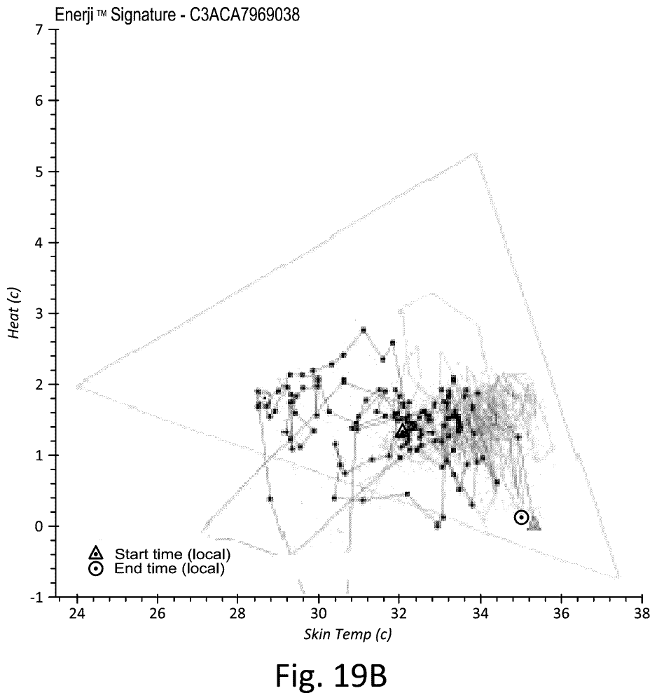

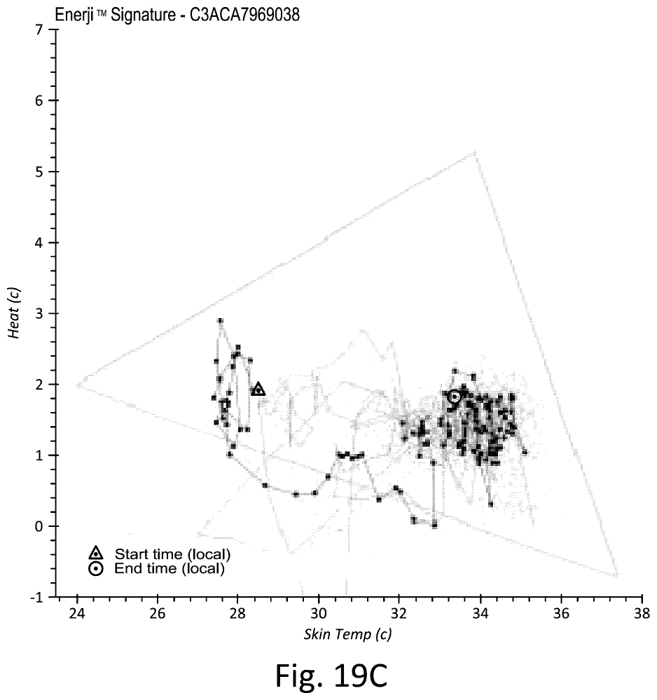

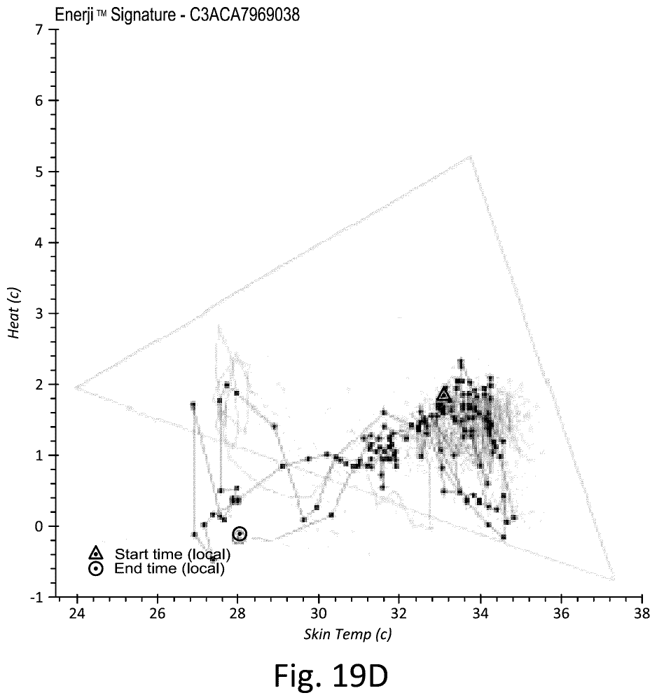

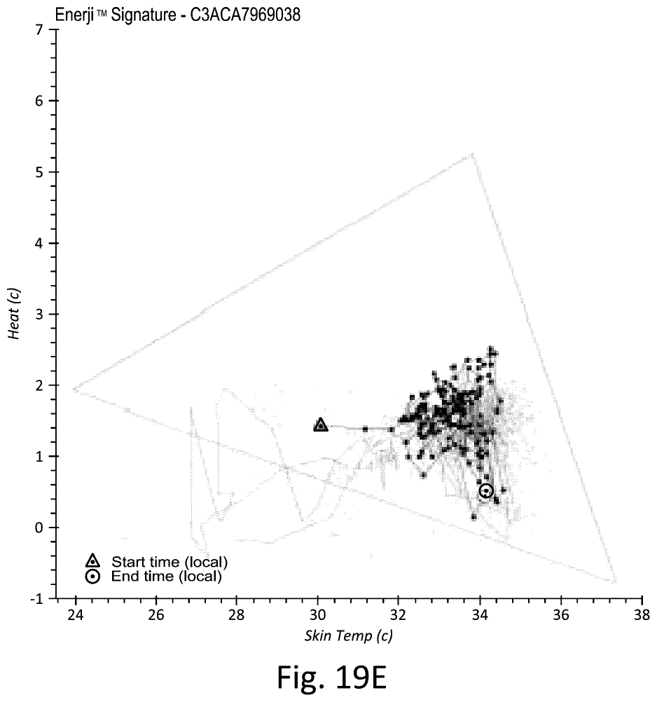

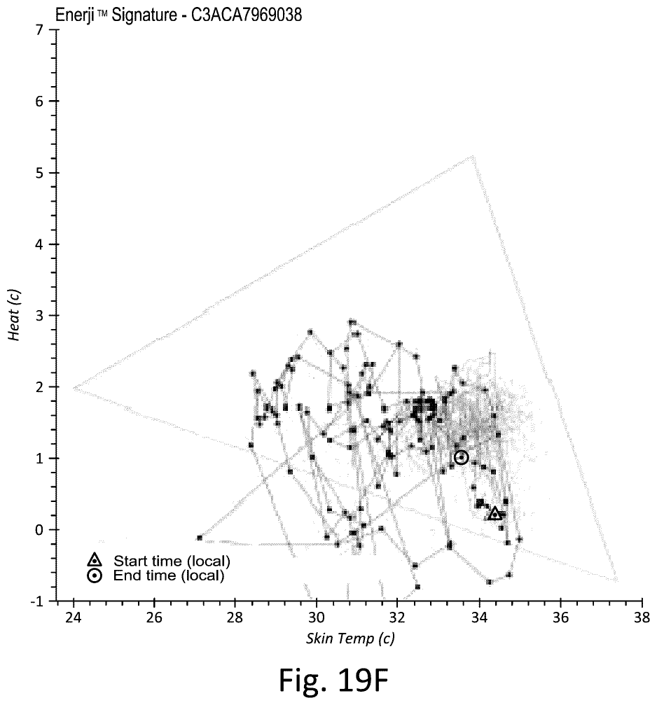

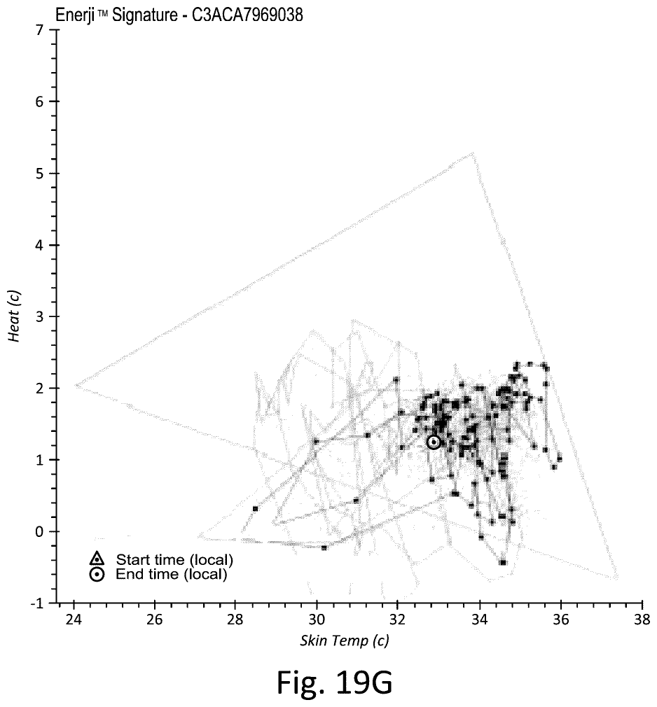

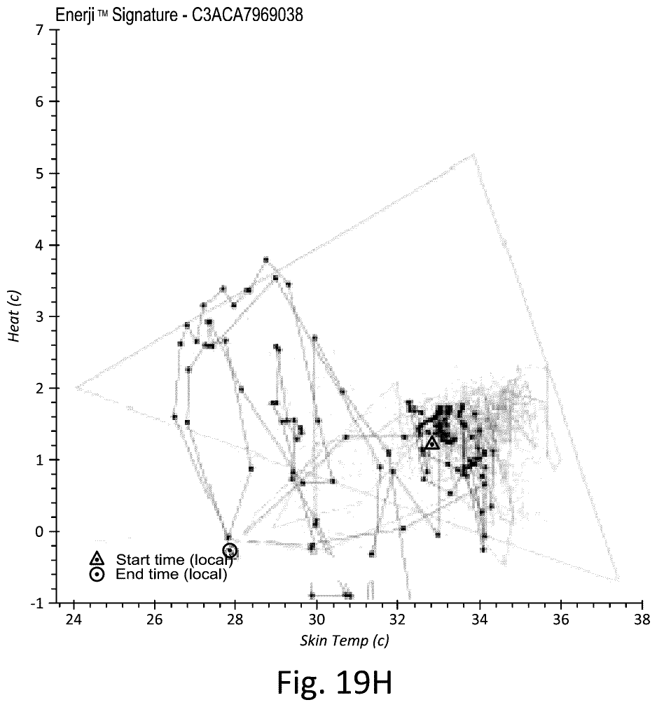

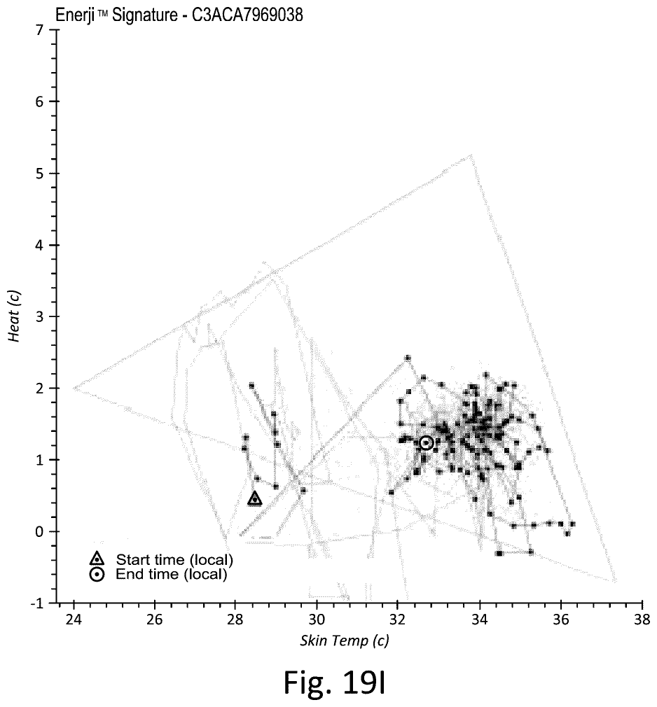

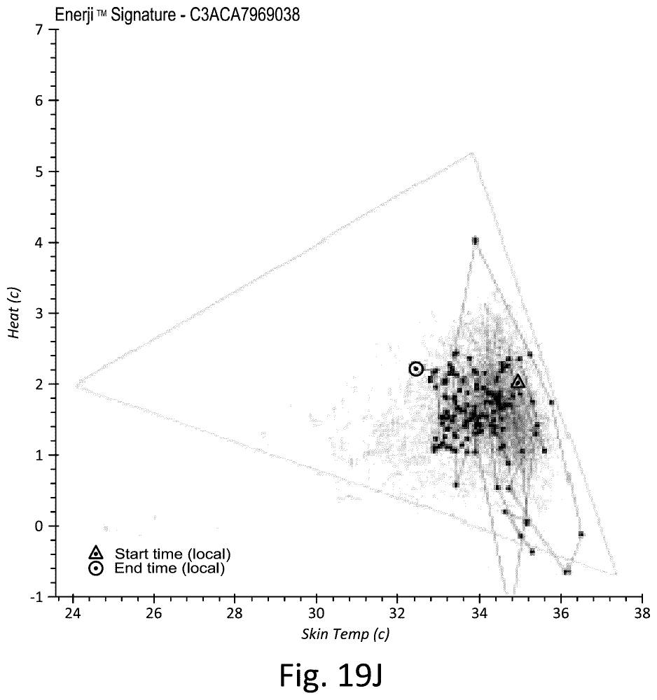

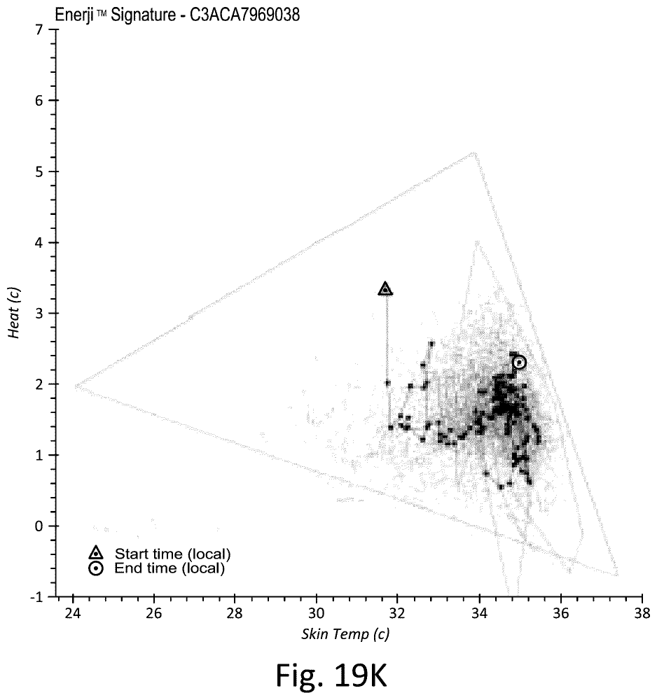

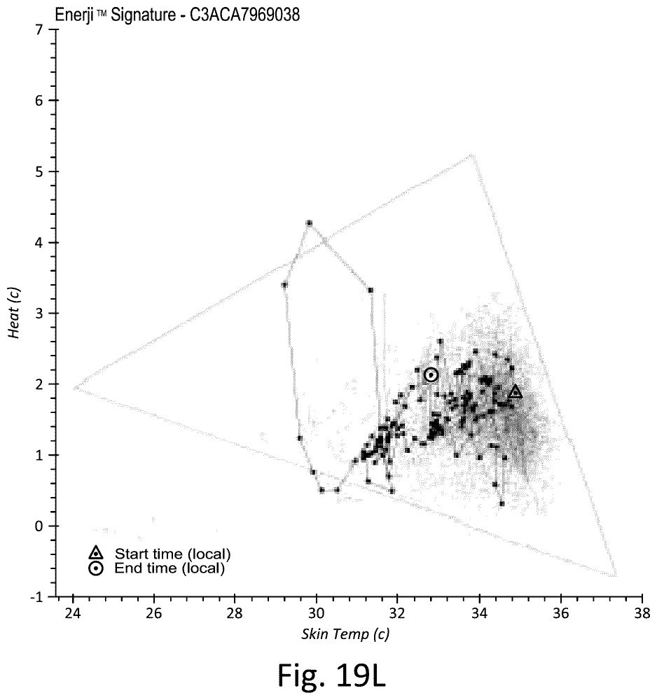

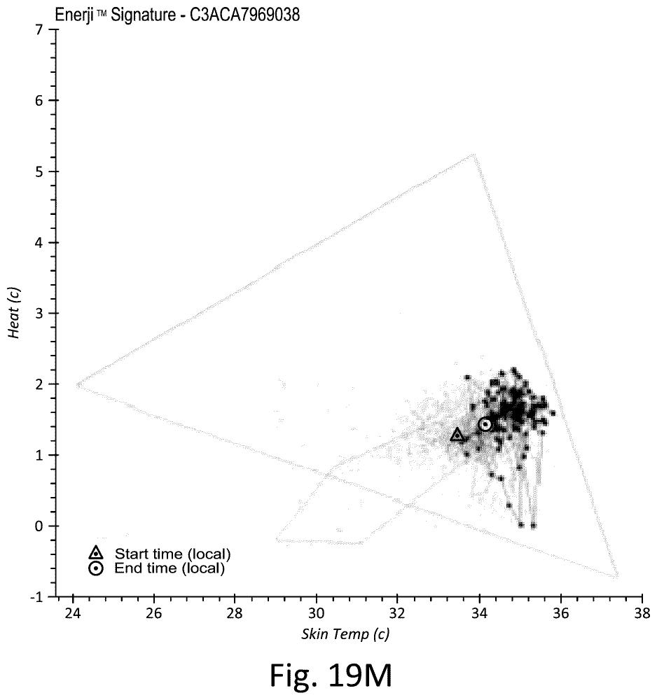

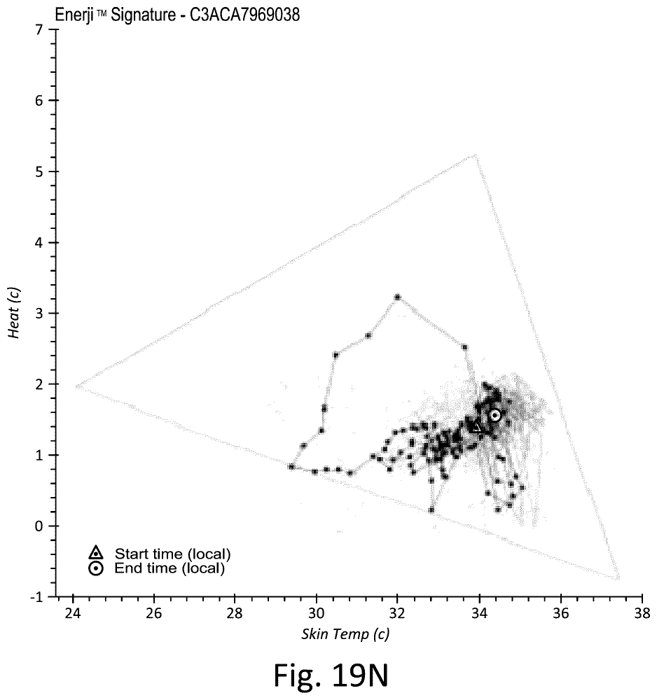

FIGS. 19A, 19B 19C, 19D, 19E, 19F, 19G, 19H, 191, 19J, 19K, 19L, 19M, 19N illustrate an example embodiment of recognizable features detected by the system for an 8 hour period over days in a subject with chronic heart failure.

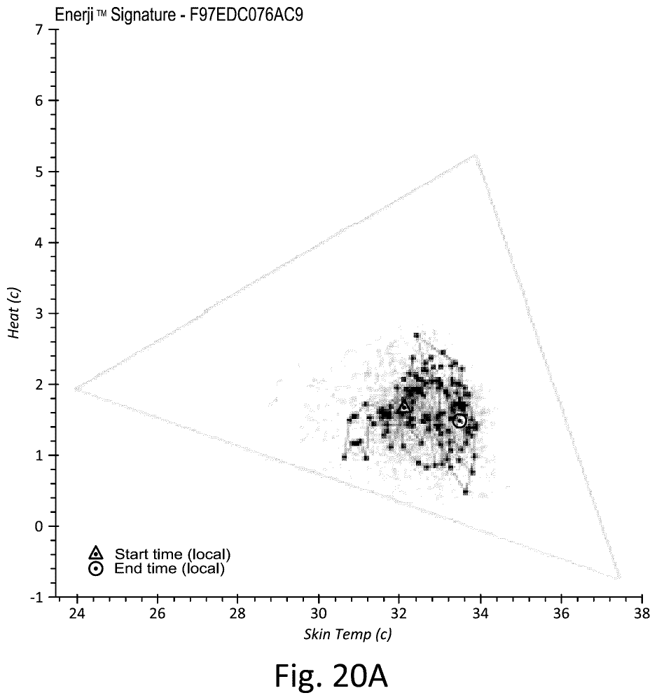

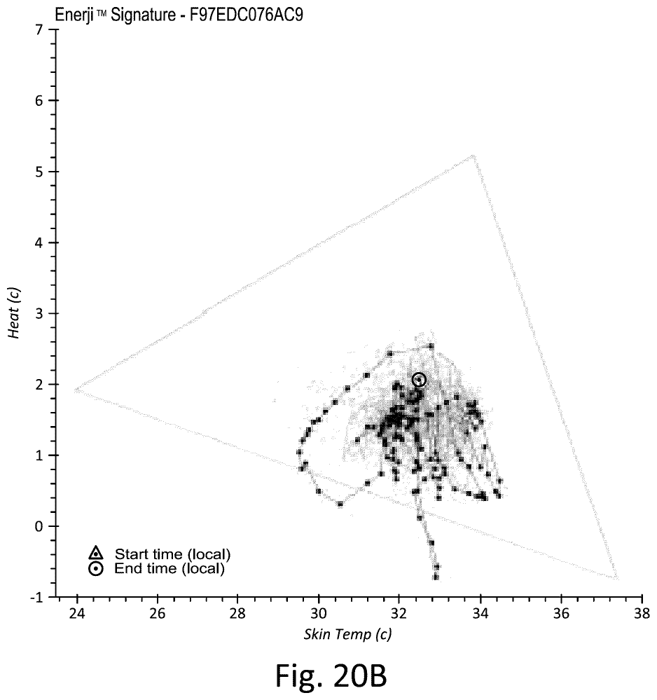

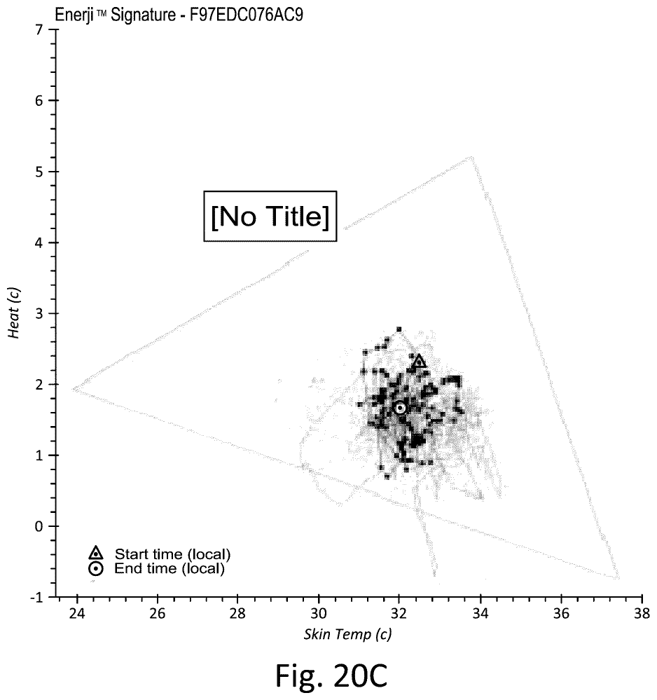

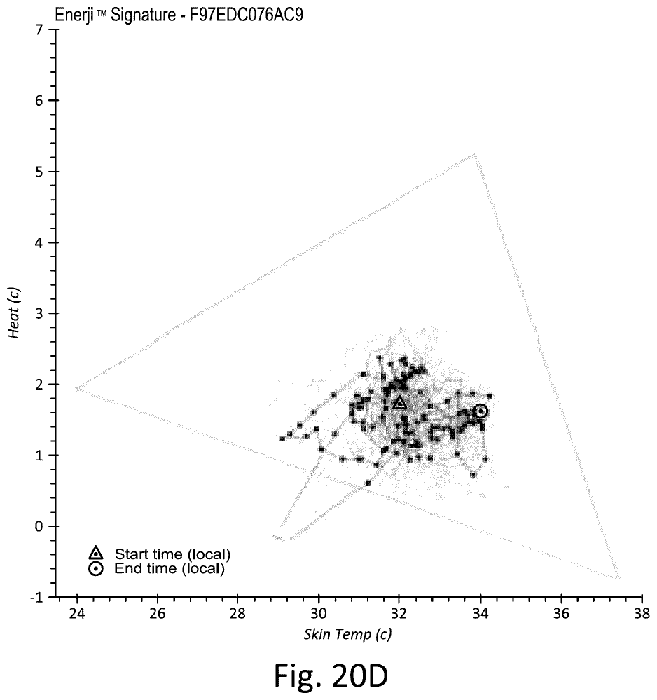

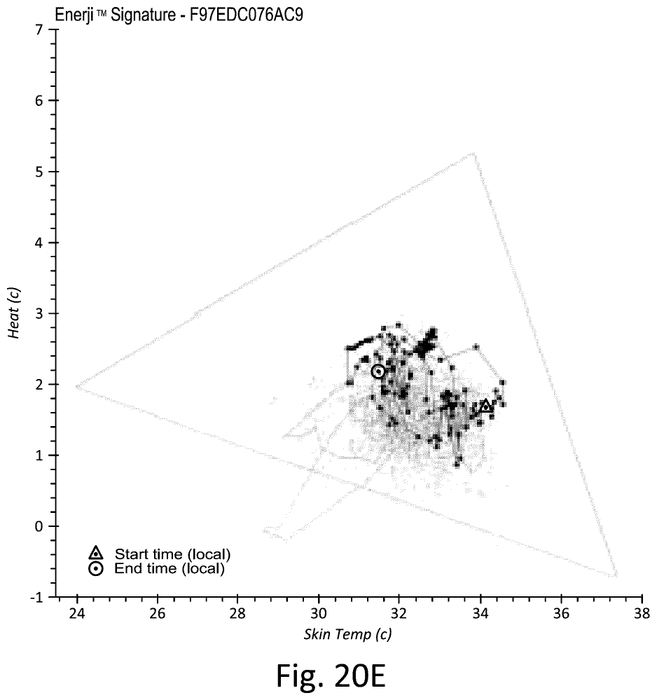

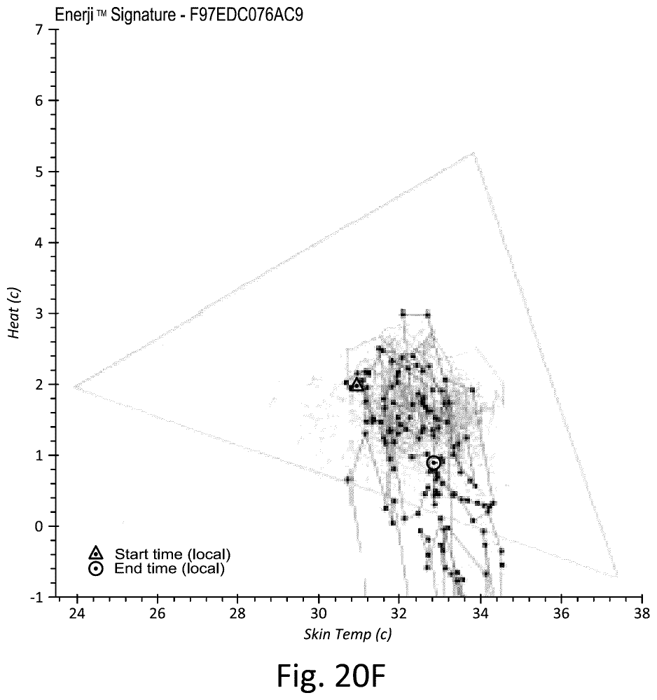

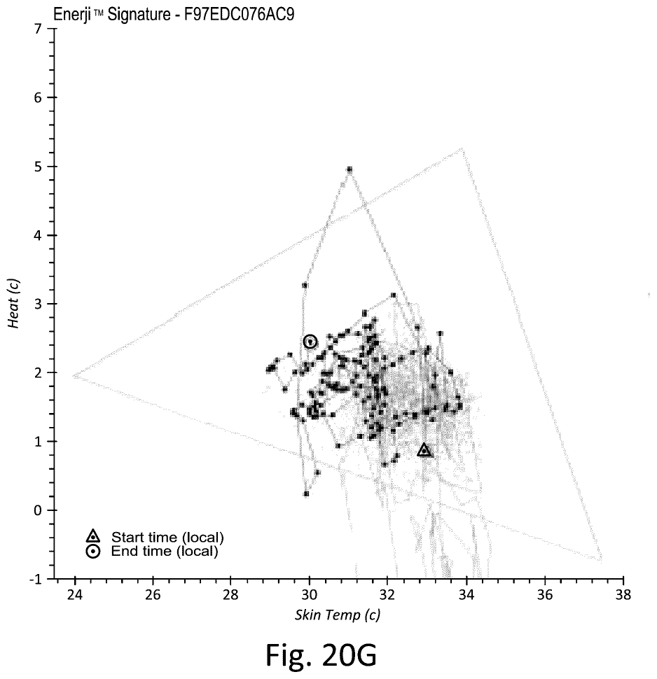

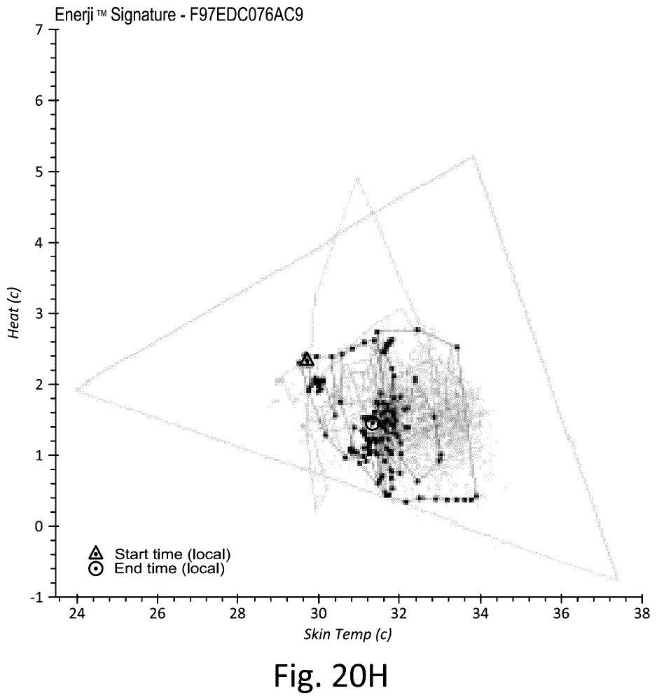

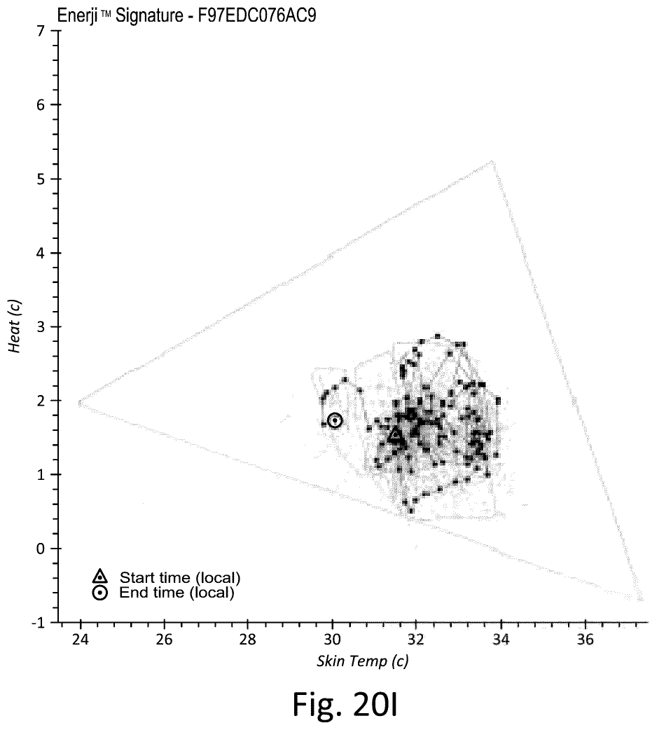

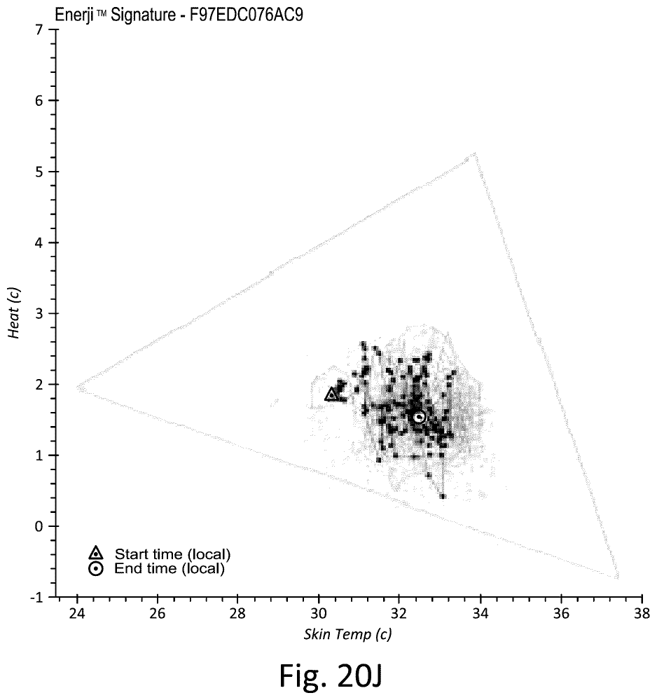

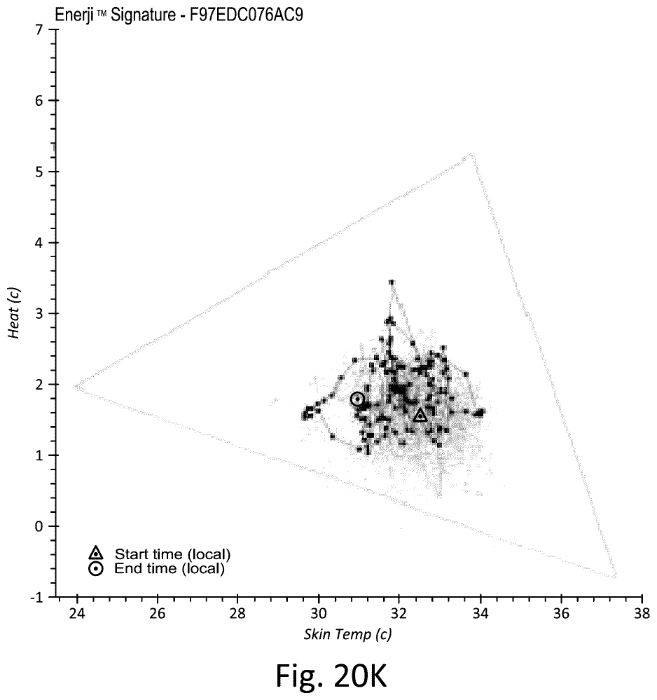

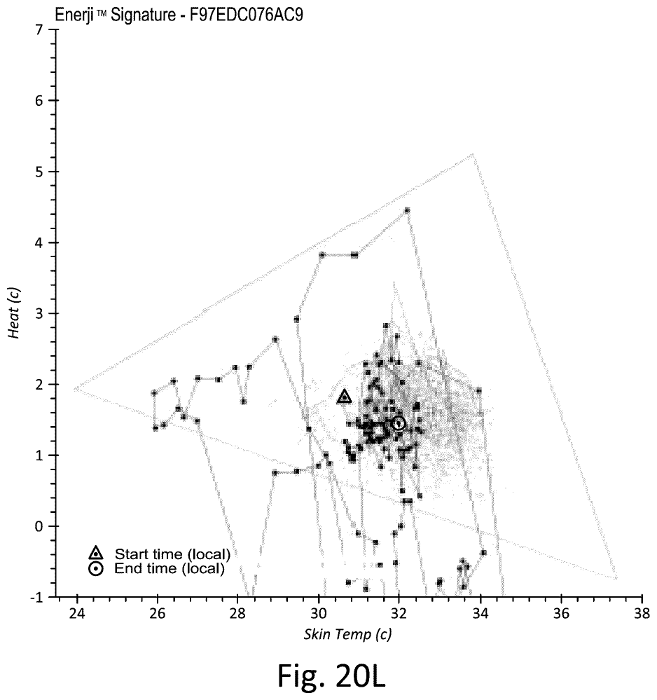

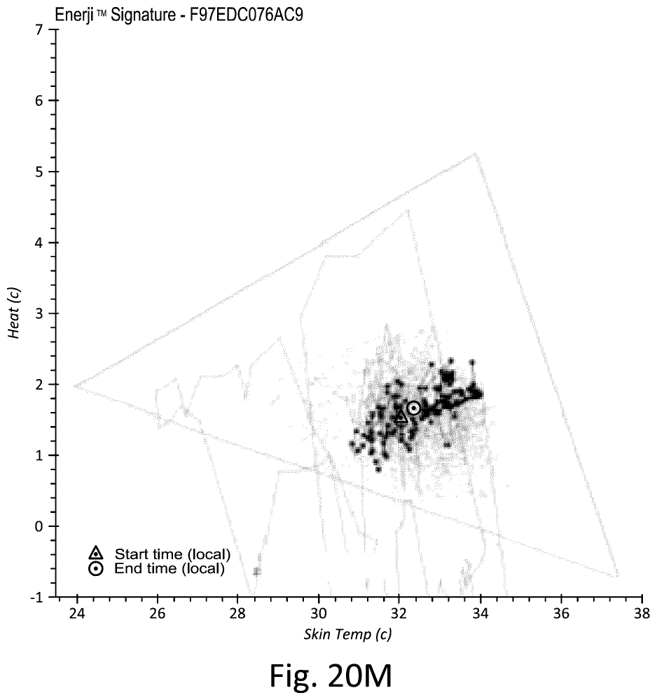

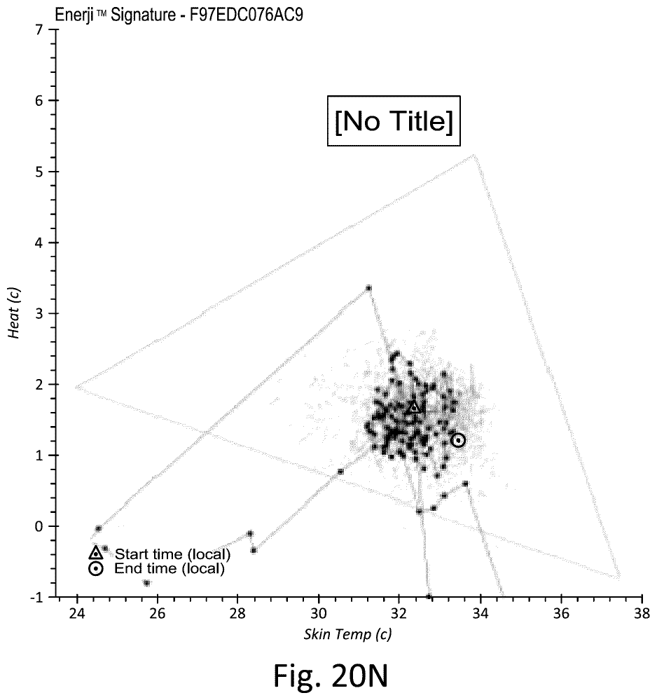

FIGS. 20A, 20B, 20C, 20D, 20E, 20F, 20G, 20H, 201, 20J, 20K, 20L, 20M, 20N illustrate an example embodiment of recognizable features detected by the system for an 8 hour period over days in a subject with chronic heart failure.

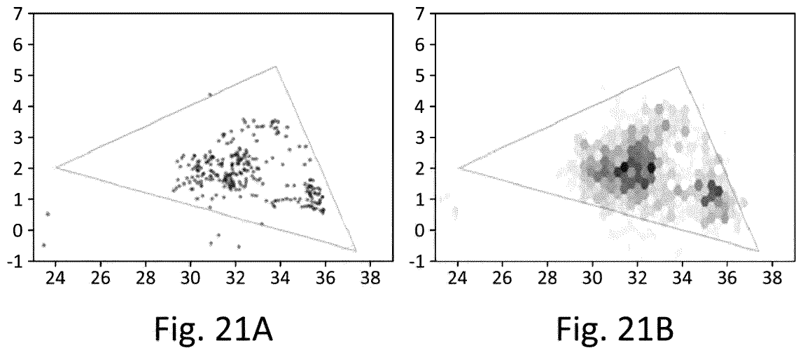

FIGS. 21A and 21B illustrate an example embodiment of plurality of thermoregulation measurements of a subject over a period of time. In some embodiments, the plurality of thermoregulation measurements may be collected with any of the devices described above. FIG. 21A shows the raw data of the plurality of thermoregulation measurements are shown external temperatures as a function of a thermoregulation measurement. FIG. 21B shows the plurality of thermoregulation measurements binned together and represented as space-filling hexagons to facilitate point-to-point distance computation. Binning of the plurality of thermoregulation measurements can allow for various geometric distance calculations. For example, binning the plurality of thermoregulation measurements into a plurality of hexagons can allow for an absolute distance between a first thermoregulation measurement of the plurality of thermoregulation measurements and a second thermoregulation measurement of the plurality of thermoregulation measurements. This can allow for a more accurate assessment of the data collected to help determine whether a thermoregulation system of a subject is resilient and/or whether intervention as to the subject is needed and the urgency of such an intervention.

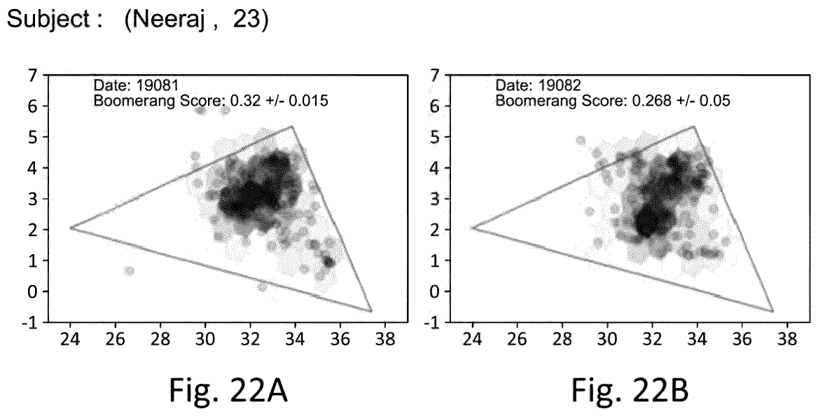

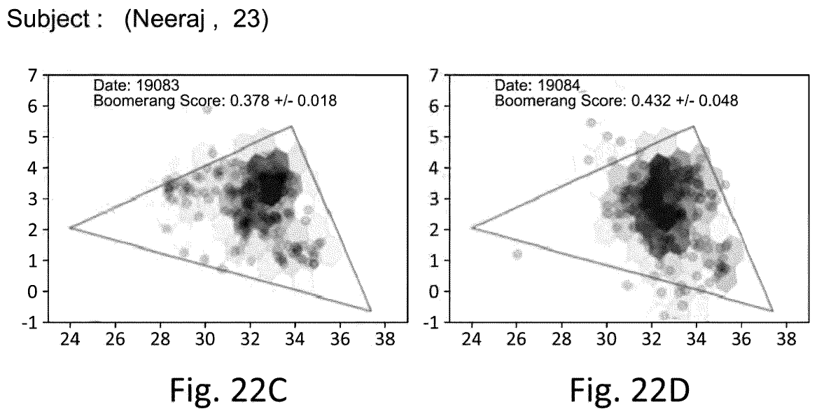

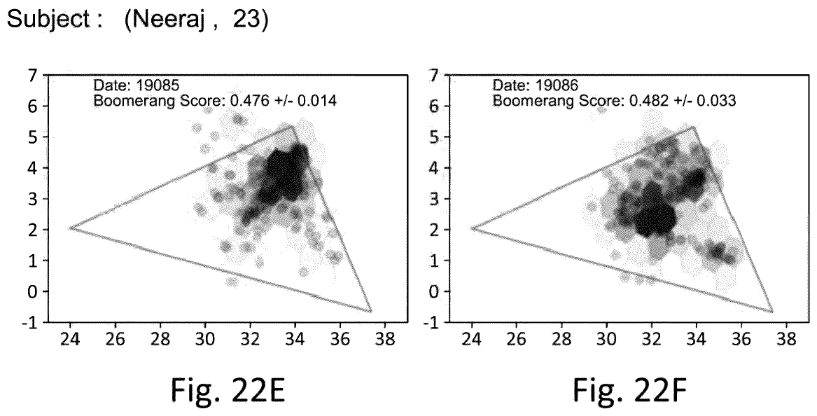

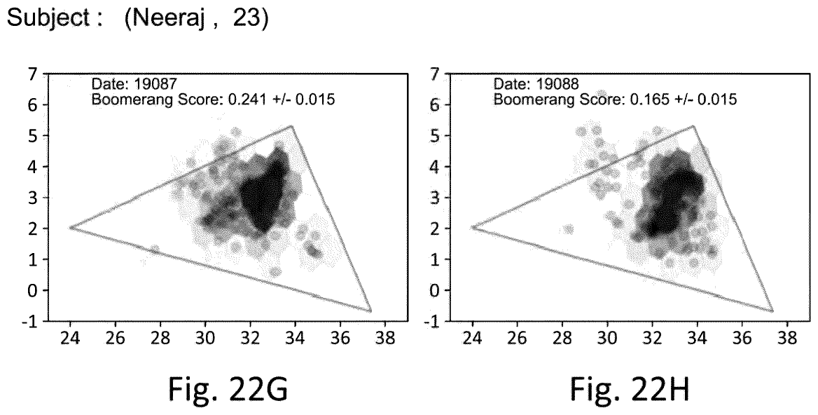

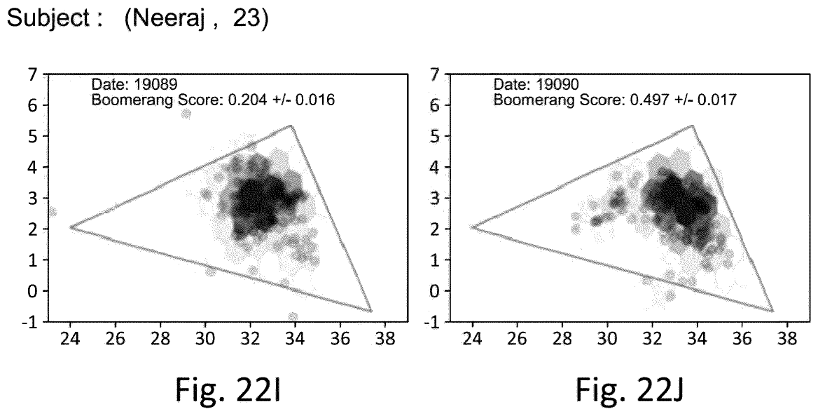

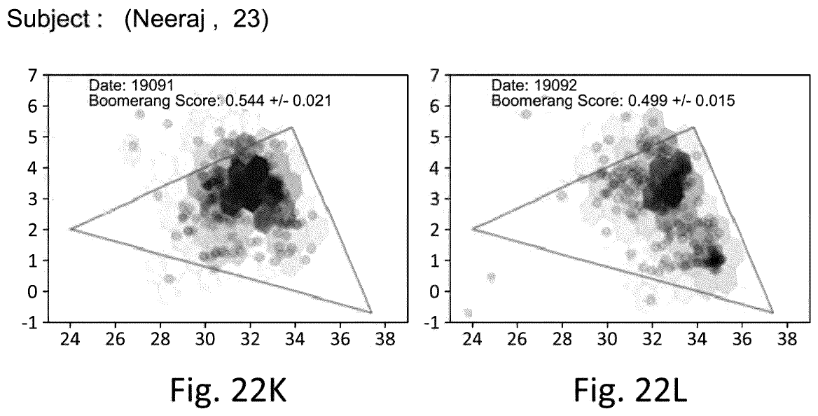

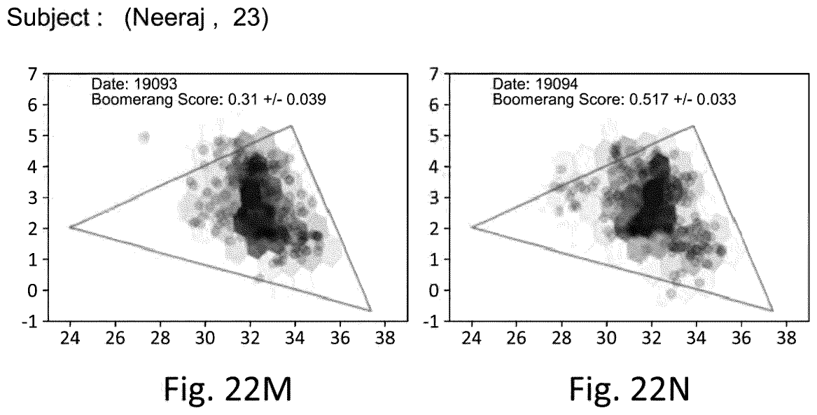

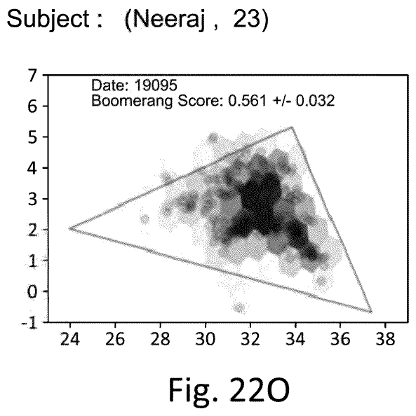

FIGS. 22A, 22B, 22C, 22D, 22E, 22F, 22G, 22H, 221, 22J, 22K, 22L, 22M, 22N, 22O illustrate an example embodiments of binned plurality of thermoregulation measurements of a subject over a period of fifteen days. FIGS. 22A-22O show that an overall recognizable pattern illustrated through the daily collection of the plurality of thermoregulation measurements may be different or vary on a daily basis. Collecting a plurality of thermoregulation measurements over a period of time on a daily basis, for example over 15 days on a daily basis, may allow for a more accurate determination as to the resilience of the thermoregulation system of a subject and/or the resilience score of the thermoregulation system of the subject. The resilience score may be calculated by first determining the probability of a binned thermoregulation measurement moving and therefore changing the absolute difference between the thermoregulation measurement and another thermoregulation measurement. The change in absolute difference between a first thermoregulation measurement and a second thermoregulation measurement may change the recognizable pattern of the overall plurality of thermoregulation measurements. For example, the change in absolute difference may cause the recognizable pattern to become more ordered or more unordered based on the change. Based on the orderliness of the recognizable pattern, artificial intelligence can determine the similarities and differences between the orderliness of the recognizable pattern of the plurality of thermoregulation measurements of the subject and the orderliness of at least one model recognizable pattern. Artificial intelligence can determine the probability the orderliness of the recognizable pattern of the plurality of thermoregulation measurements of the subject is like the orderliness of at least one of the model recognizable patterns within a confidence level. For example, the closer the determined probability is to 1, the more like the orderliness of the recognizable pattern of the plurality of thermoregulation measurements of the subject is to the model recognizable pattern that implies a high functioning and/or efficient thermoregulation system. This may lead to a higher thermoregulation resilience score. For example, the closer the determined probability is to 0, the less like the orderliness of the recognizable pattern of the plurality of thermoregulation measurements of the subject is to the model recognizable pattern that implies a high functioning and/or efficient thermoregulation system. The less like the orderliness of the recognizable pattern of the plurality of thermoregulation measurements of the subject is to the model recognizable pattern that implies a high functioning and/or efficient thermoregulation system, the lower functioning the thermoregulation system is of the subject. This may lead to a lower thermoregulation resilience score.

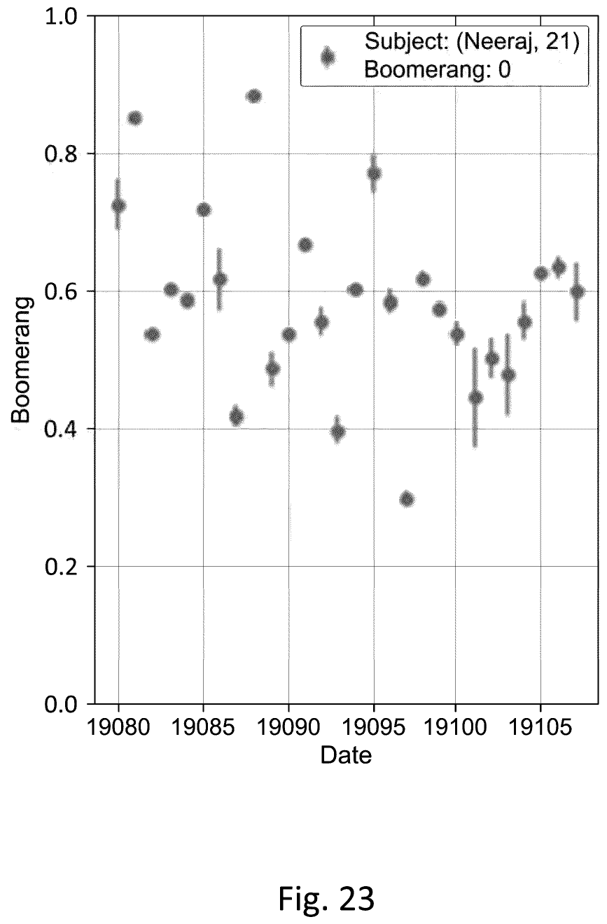

FIG. 23 illustrates an example embodiment of a plurality thermoregulation resilience scores of a subject over a period of time defined as one month. As shown, the graph illustrates the date and the subject's corresponding thermoregulation resilience score on that date. As further shown, the thermoregulation resilience score increases and decreases over the period of time defined as one month. The greater range and variety of thermoregulation resilience scores determined over the period of time, the greater change in the subject's health status on a day-to-day basis. This may be related to a periodicity of the subject's health status. A periodicity of the subject's health status can correlate to a more positive health status of a subject or a more negative health status of a subject.

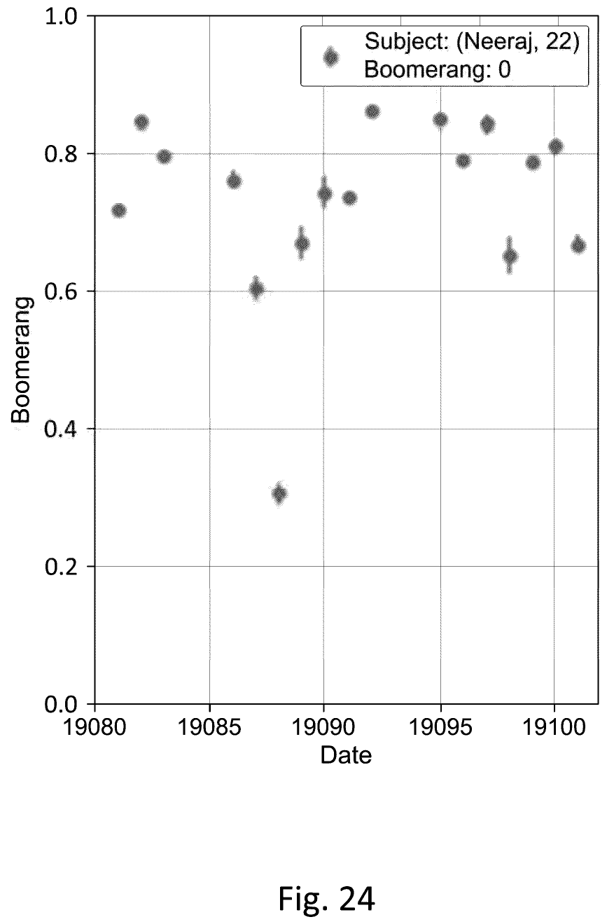

FIG. 24 illustrates an example embodiment of a plurality thermoregulation resilience scores of a subject over a period of time defined as one month. As shown, the graph illustrates the date and the subject's corresponding thermoregulation resilience score on that date. As shown, the thermoregulation resilience score remains relatively constant over the period of time defined as one month. The less range and variety of thermoregulation resilience scores determined over the period of time, the less change in the subject's health status on a day-to-day basis. As shown, the subject has a consistent relatively high thermoregulation resilience score. In other words, the thermoregulation resilience scores are close to 1. This may mean the subject generally had positive health status on a day-to-day basis over the period of time.

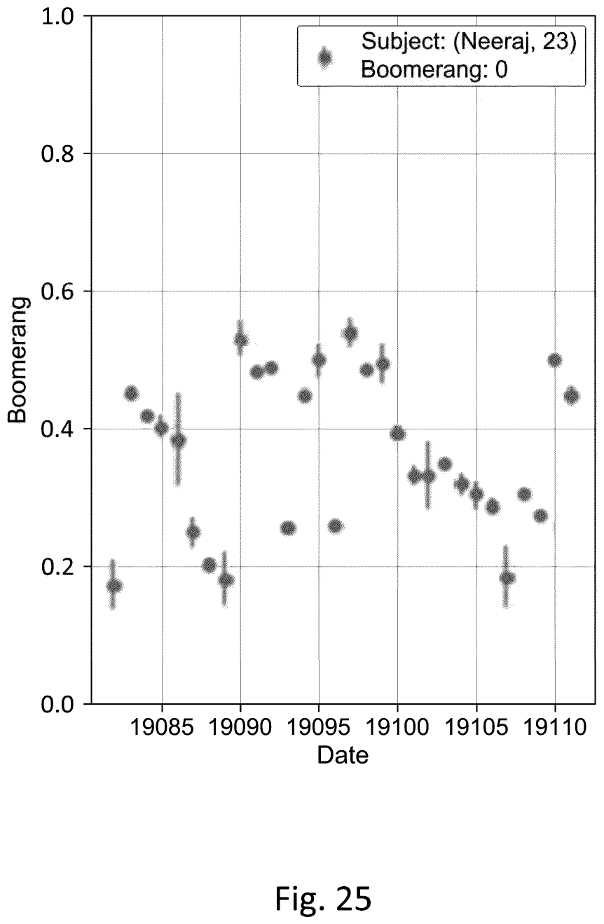

FIG. 25 illustrates an example embodiment of a plurality thermoregulation resilience scores of a subject over a period of time defined as one month. As shown, the graph illustrates the date and the subject's corresponding thermoregulation resilience score on that date. As shown, the thermoregulation resilience score remains relatively constant over the period of time defined as one month. The less range and variety of thermoregulation resilience scores determined over the period of time, the less change in the subject's health status on a day-to-day basis. As shown, the subject has a consistent relatively low thermoregulation resilience score. In other words, the thermoregulation resilience scores are closer to 0 than to 1. This may mean the subject generally had negative health status on a day-to-day basis over the period of time.

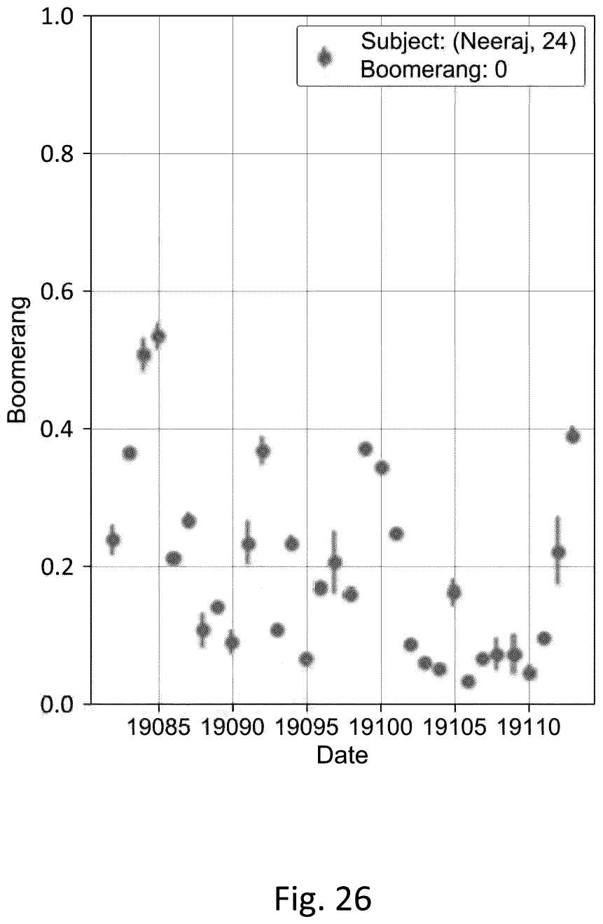

FIG. 26 illustrates an example embodiment of a plurality thermoregulation resilience scores of a subject over a period of time defined as one month. As shown, the graph illustrates the date and the subject's corresponding thermoregulation resilience score on that date. As shown, the thermoregulation resilience score remains relatively constant over the period of time defined as one month. The less range and variety of thermoregulation resilience scores determined over the period of time, the less change in the subject's health status on a day-to-day basis. As shown, the subject has a consistent relatively low thermoregulation resilience score. In other words, the thermoregulation resilience scores are closer to 0 than to 1. This may mean the subject generally had negative health status on a day-to-day basis over the period of time.

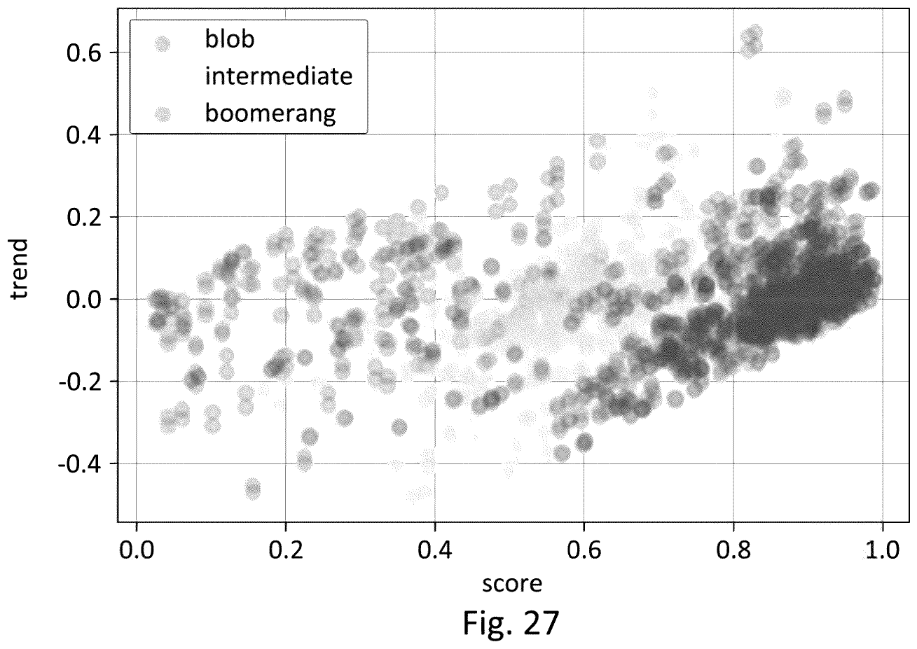

FIG. 27 illustrates an example embodiment of a plurality of thermoregulation measurements of three subjects over a period of time defined as one day. As shown, a first subject is represented by the red data points, a second subject is represented by the yellow data points, and a third subject is represented by the green data points. As shown in FIG. 27, the thermoregulation resilience score of the subject can change throughout the day. As further shown in FIG. 27, while the thermoregulation resilience score may change, the trend, direction, or pattern of which the thermoregulation resilience score changes along remain the similar or consistent throughout the day. For example, the first subject has a range of thermoregulation resilience scores through the day which may lead to an assessment resulting in determination of the need for intervention as to the subject or a determination of a health status that may be negative. As shown in FIG. 27, for example, the first subject may have various thermoregulation resilience scores throughout the day, but the first subject generally remains in a first range of thermoregulation resilience scores. The first range of thermoregulation resilience scores for the first subject, as shown, is different than a second range of thermoregulation resilience scores for the second subject, which is different than a third range of thermoregulation resilience scores for the third subject. This can allow the thermoregulation system of a subject to be related to a phenotype. In this manner, any sensor sensing a biological measurement can be used in the assessment of the thermoregulation system of a subject. The thermoregulation resilience scores of the first subject may relate to a recognizable pattern that correlates to a negative health status. Similarly, as shown in FIG. 27, for example, the second subject may have various thermoregulation resilience scores throughout the day, but the second subject generally remains in the second range of thermoregulation resilience scores. The thermoregulation resilience scores of the second subject may relate to a recognizable pattern that correlates to an average health status. Similarly, as shown in FIG. 27, for example, the third subject may have various thermoregulation resilience scores throughout the day, but the third subject generally remains in the third range of thermoregulation resilience scores. The thermoregulation resilience scores of the third subject may relate to a recognizable pattern that correlates to an positive health status.

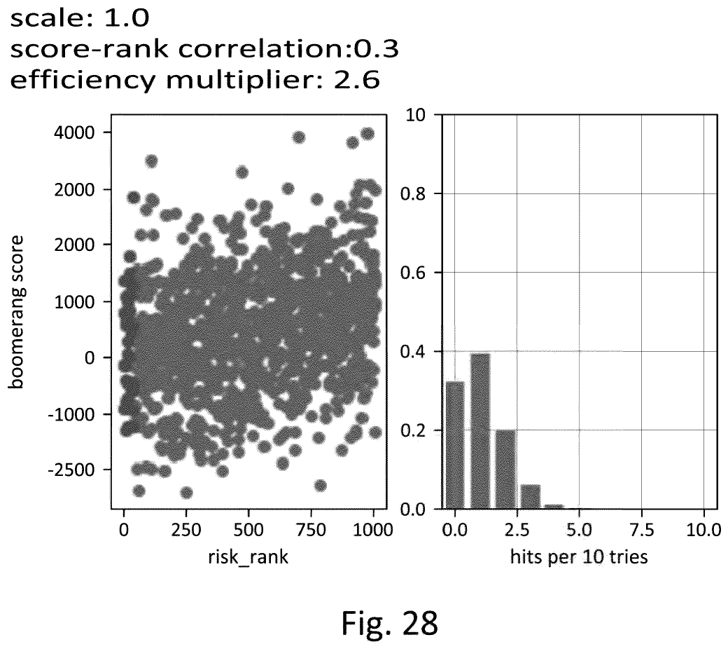

FIG. 28 illustrates an example embodiment of a plurality of thermoregulation resilience scores of a plurality of individuals plotted as a function of a combination of a plurality of thermoregulation resilience scores and a plurality of other biological measurements. This combination may be considered the risk rank. This plot may be used to identify a subject and/or assess the risk of the subject having a health status that may require treatment or intervention. As shown in FIG. 28, the majority of data points are in the range of approximately −1000 to 2000 thermoregulation resilience scores. The lower the risk rank, the higher risk a subject is at for a health status that may be considered at risk for treatment or intervention. As further shown in FIG. 28, by assessing the risk of a subject in this manner, the probability of assessing the risk of the subject correctly is 5-25% and the efficiency multiplier is 2.6.

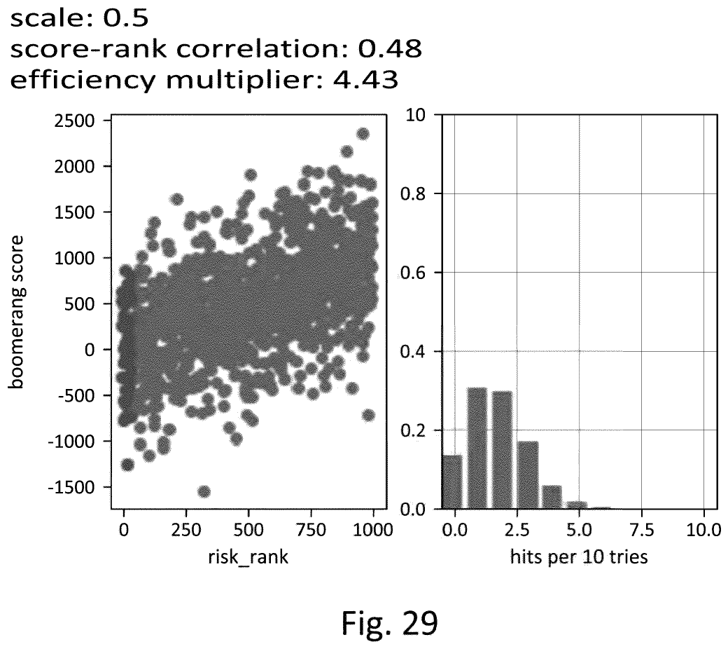

FIG. 29 illustrates an example embodiment of a plurality of thermoregulation resilience scores (distinct from those score depicted in FIG. 28) of a plurality of individuals plotted as a function of a combination of a plurality of thermoregulation resilience scores and a plurality of other biological measurements. This combination may be considered the risk rank. This plot may be used to identify a subject and/or assess the risk of the subject having a health status that may require treatment or intervention. As shown in FIG. 29, at least one outlier is removed from plot, therefore allowing the majority of data points to be in the range of approximately −500 to 1000 thermoregulation resilience scores. The lower the risk rank, the higher risk a subject is at for a health status that may be considered at risk for treatment or intervention. As further shown in FIG. 29, by assessing the risk of a subject in this manner, the probability of assessing the risk of the subject correctly is 5-30% and the efficiency multiplier is 4.43. As shown in FIG. 29, by removing the outliers of the data set, the probability of correctly assessing the risk of the subject is increased.

According to the examples depicted in FIG. 28 or 29, wearable device disclosed herein may be configured to provide precise information for a single individual to improve that individual's medical care or optimize lifestyle. Alternatively, a plurality of wearable device may be distributed across a large cohort to gain operating efficiencies even though the devices does not have sufficient precision guide care on a single individual. These embodiments address various key problems with the existing art. For example, the existing health care industry has limiting established practices, involves large populations which are subject to a low rate of high-cost events; minimal effort are made to intervene with these events because the cost of prediction exceeds the value generated from prevention; mitigation strategies are executed at the population level (i.e. health guidance stratified by demographics) rather than at the individual level; need utility requires the ability to efficiently segment population to improve odds of event accurate prediction. This embodiment there yield superior health and financial outcomes as described in the following illustrative Case Examples:

### Case Example 1: Serious Medical Events in Fragile Elderly

Constant monitoring by skilled medical professional can prevent many hospitalizations in elderly. Cost of skilled outpatient care is prohibitive at scale. An IoT solution which improves the relative odds of predicting a medical event can be used to triage outpatient care resources. This triage function multiplies the effective capacity of skilled nurses. Superior outcomes include decrease is morbidity associated with aging and decrease in costs related to hospitalization.

### Case Example 2: Medical Insurance for Healthy Young Population

Young people need minimal medical care except in the case of traumatic injury or unexpected disease. Demographic parameters determine insurance rates. But unknown physiologic risk factors include the abilities to usefully segment subgroups of different risk and incentivize screening of riskiest group and to identify stressed days where injury is more likely and incentivize customer to avoid unnecessary risk. Superior outcomes include: Reduction of morbidity associated with accidents and preventable disease and cost reduction for insurers and customers.

### Case 3: Heat Stroke in Laborers

Working in hot conditions is risky, particularly agricultural or logistics. Most businesses have an all-or-nothing strategy. All workers labor until the temperature exceeds a threshold, then everyone stops. Identifying a individuals who are most at risk of heat stroke, will allow targeted work management. Superior outcomes include: less downtime and less cost from injury.

1. A system configured to identify at least one recognizable feature associated with a health state of a subject, the system comprising:
at least one sensor for measuring a heat flux of said subject and generating a data stream therefrom, wherein said data stream includes a plurality of heat flux measurements; and
a processor configured to receive the data stream and recognize said at least one recognizable feature associated with said health state.

2. The system of claim 1, wherein the at least one sensor for measuring heat flux comprises at least one sensor array for simultaneously measuring heat elimination by said subject and an environmental temperature proximal to said subject.

3. The system of claim 1, wherein the plurality of heat flux measurements comprise a plurality of heat elimination measurements expressed as a function of environmental temperature.

4. The system of claim 1 further comprising at least one transition state corresponding to the at least one recognizable feature of the data stream.

5. The system of claim 1, wherein the at least one recognizable feature (i) is associated with a health state corresponds to a biological response, (ii) is indicative of thermal regulation, or (iii) corresponds to a homeostatic state of the subject.

6. (canceled)

7. (canceled)

8. (canceled)

9. The system of claim 1, wherein the data stream further comprises at least one work quantification associated with said plurality of heat flux measurements.

10. (canceled)

11. (canceled)

12. (canceled)

13. The system of claim 1, further configured to identify: (i) at least one motif, wherein the at least one motif comprises a plurality of heat flux measurements, (ii) at least one discord comprising a plurality of heat flux measurements that do not correspond to the at least one recognizable feature, or (iii) a difference between a first heat flux measurement of the plurality of heat flux measurements and a second heat flux measurement of the plurality of heat flux measurements.

14. (canceled)

15. (canceled)

16. (canceled)

17. (canceled)

18. (canceled)

19. (canceled)

20. (canceled)

21. The system of claim 1, wherein the processor forecasts: (i) a plurality of future heat flux measurements based on the at least one recognizable feature identified, or (ii) a future recognizable feature based on the at least one recognizable feature identified.

22. (canceled)

23. The system of claim 1, wherein the processor determines the subject is an anomaly of a population based on the at least one recognizable feature.

24. (canceled)

25. (canceled)

26. The system of claim 1, wherein the processor generates a recommended counter action based on the at least one recognizable feature.

27. The system of claim 26, wherein the recommended counter action comprises at least one of:
apply cold compress;
rest;
engage in physical activity;
cease administration of a medicament;
commence administration of a medicament;
go inside;
go outside;
increase hydration;
decrease hydration; and
elevate one or more extremity.

28. The system of claim 1, wherein the processor is configured to analyze the data stream.

29. A method of identifying at least one recognizable feature associated with a biological response comprising:
measuring heat flux of a subject;
generating a data stream wherein said data stream includes a plurality of heat flux measurements; and
recognizing said at least one recognizable feature from said data stream.

30. The method of claim 29, further comprising comparing analysis of the at least one recognizable feature identified for a first subject to the analysis of the at least one recognizable feature identified for a second subject.

31. A method of assessing resilience of thermoregulation in a subject and generating a recommendation to engage in an intervention as to the subject based on the assessed resilience of thermoregulation, the method comprising:
generating a thermoregulation resilience score for the subject over a pre-determined time period, wherein the thermoregulation resilience score is based on a data stream comprising a plurality of thermoregulation measurements of the subject;
determining whether a need for an intervention as to the subject is above a pre-determined threshold of urgency; and
generating a recommendation to engage in the intervention as to subject.

32. (canceled)

33. (canceled)

34. (canceled)

35. (canceled)

36. (canceled)

37. (canceled)

38. (canceled)

39. (canceled)

40. (canceled)

42. The method of claim 31, further comprising alerting a medical practitioner that the recommendation has been generated.

43. (canceled)

44. (canceled)

45. (canceled)

46. (canceled)

47. (canceled)

48. (canceled)

49. (canceled)

50. (canceled)

51. (canceled)

52. (canceled)

53. A method of identifying a subject in need of urgent intervention, the method comprising:
measuring a plurality of thermoregulation measurement of the subject;
generating a data stream, over a time period, wherein the data stream includes a plurality of thermoregulation measurements;
generating a score, for the time period, based on the data stream;
identifying whether the subject is in urgent need of intervention; and
sending an alert relating to the urgent need for intervention.

54. (canceled)

55. (canceled)

56. An alert system configured to generate an alert to intervene as to the well-being of a subject, the alert system comprising:
at least one sensor for generating a plurality of thermoregulation measurements of the subject;
a communication means for generating a data stream of thermoregulation measurements, wherein the data stream includes a plurality of thermoregulation measurements over a pre-determined time period; and
a processor configured to receive the data stream and generate a score, associated with the pre-determined time period, based on the resilience of the thermoregulation measurement of the subject.

57. (canceled)

58. (canceled)

59. (canceled)

60. A method of identifying a subject in need of an intervention and alerting to another relating to the need for intervention, the method comprising:
measuring a plurality of thermoregulation measurements of the subject;
generating a data stream, over a time period, wherein the data stream includes a plurality of thermoregulation measurements;
generating a score, for the time period, based on the data stream;
grouping the score and a plurality of other biological measurements into a data set;
identifying whether the subject is in need of intervention based at least in part on the score; and
sending an alert relating to the need for intervention.

61. (canceled)

62. (canceled)

63. (canceled)

64. A method of identifying a patient having a health risk, the method comprising:
measuring a plurality of thermoregulation measurement of the subject;
generating a data stream, over a time period, wherein the data stream includes a plurality of thermoregulation measurements;
generating a score, for the time period, based on the data stream;
grouping the score and a plurality of other biological measurements into a data set; and
identifying whether the subject has a health risk.

65. (canceled)
